(19) United States
(12) Patent Application Publication
(10) Pub. No.: US 2008/0300292 A1
(43) Pub. Date: Dec. 4, 2008

(54) Nitrosated and Nitrosylated Compounds, Compositions and Methods for the Treatment of Ophthalmic Disorders

(75) Inventors: L. Gordon Letts, Dover, MA (US); David S. Garvey, Dover, MA (US)

Correspondence Address: WILMERHALE/NITROMED, 1875 PENNSYLVANIA AVE, NW, WASHINGTON, DC, 20006, US

(73) Assignee: NitroMed, Inc, Lexington, MA (US)

(21) Appl. No.: 11/667,272

(22) PCT Filed: Nov. 8, 2005
(86) PCT No.: PCT/US05/40314

§ 371 Date: Mar. 21, 2008

Related U.S. Application Data
(60) Provisional application No. 60/625,578, filed on Nov. 8, 2004.

(51) Int. Cl. A61K 31/4025; A61P 27/12; A61P 27/06

(52) U.S. Cl. 514/422

(57) ABSTRACT

The invention describes novel nitrosated and/or nitrosylated compounds or pharmaceutically acceptable salts thereof, and novel compositions comprising at least one nitrosated and/or nitrosylated compound, and, optionally, at least one nitric oxide donor and/or at least one therapeutic agent. The invention also provides novel compositions and kits comprising at least one compound of the invention, that is optionally nitrosated and/or nitrosylated, and, optionally, at least one nitric oxide donor compound and/or at least one therapeutic agent. The invention also provides methods for treating ophthalmic disorders. The nitrosated and/or nitrosylated compounds are preferably nitrosated and/or nitrosylated (3-adrenergic antagonists and nitrosated and/or nitrosylated angiotensin-converting enzyme (ACE) inhibitors.

### RELATED APPLICATION

This application claims priority under 35 USC § 119 to U.S. Application No. 60/625,578 filed Nov. 8, 2004, which is herein incorporated by reference in its entirety.

### FIELD OF THE INVENTION

The invention describes novel nitrosated and/or nitrosylated compounds or pharmaceutically acceptable salts thereof, and novel compositions comprising at least one nitrosated and/or nitrosylated compound, and, optionally, at least one nitric oxide donor and/or at least one therapeutic agent. The invention also provides novel compositions and kits comprising at least one compound of the invention, that is optionally nitrosated and/or nitrosylated, and, optionally, at least one nitric oxide donor compound and/or at least one therapeutic agent. The invention also provides methods for treating ophthalmic disorders. The nitrosated and/or nitrosylated compounds are preferably nitrosated and/or nitrosylated β-adrenergic antagonists and nitrosated and/or nitrosylated angiotensin-converting enzyme (ACE) inhibitors.

### BACKGROUND OF THE INVENTION

Most drugs conventionally used to treat ophthalmic disorders have potentially serious side effects such as blurring of vision and other visual side effects which may lead either to decreased patient compliance or to the termination of therapy. Occasionally systemically administered drugs can also cause serious side effects, such as nausea, dyspepsia, fatigue, and metabolic acidosis, which affect patient compliance and/or necessitate the termination of treatment. Additionally, some β-adrenergic antagonists have increasingly become associated with serious pulmonary side effects attributable to their effects on β-2 receptors in pulmonary tissue. Hence there is a need in the art for compounds that have improved efficacy, lower toxicity and/or fewer side effects and that can be used at low dosages. The invention is directed to these, as well as other, important ends.

### SUMMARY OF THE INVENTION

The invention provides novel compounds that are substituted with at least one NO and/or NO₂ group (i.e., nitrosylated and/or nitrosated), and pharmaceutically acceptable salts thereof. The compounds can be, for example, β-adrenergic antagonists or ACE inhibitors. The compounds can be nitrosated and/or nitrosylated through one or more sites such as oxygen (hydroxyl condensation), sulfur (sulfhydryl condensation) and/or nitrogen. The invention also provides compositions comprising the novel compounds described herein in a pharmaceutically acceptable carrier.

The invention is also based on the discovery that administering at least one compound of the invention or a pharmaceutically acceptable salt thereof, that is optionally substituted with at least one NO and/or NO₂ group (i.e., nitrosylated and/or nitrosated), and, optionally, at least one nitric oxide donor improves the properties of the compound. Nitric oxide donors include, for example, S-nitrosothiols, nitrites, nitrates, N-oxo-N-nitrosamines, furoxans, sydnonimines, SPM 3672, SPM 5185, SPM 5186 and analogues thereof, and substrates of the various isozymes of nitric oxide synthase. Thus, another embodiment of the invention provides compositions comprising at least one compound of the invention that is optionally substituted with at least one NO and/or NO₂ group (i.e., nitrosylated and/or nitrosated), and at least one nitric oxide donor compound. The invention also provides for such compositions in a pharmaceutically acceptable carrier.

The invention provides compositions comprising at least one compound of the invention, that is optionally substituted with at least one NO and/or NO₂ group (i.e., nitrosylated and/or nitrosated), and, optionally, at least one nitric oxide donor compound and/or at least one therapeutic agent, including, but not limited to, α-adrenergic receptor agonists, α-adrenergic receptor antagonists, angiotensin-converting enzyme (ACE) inhibitors, antimicrobial compounds, antioxidants, β-adrenergic antagonists, carbonic anhydrase inhibitors, hydralazine compounds, nonsteroidal antiinflammatory compounds (NSAIDs), prostaglandins, selective cyclooxygenase-2 (COX-2) inhibitors, steroids, and combinations of two or more thereof. In a preferred embodiment the at least one therapeutic agent is selected from the group consisting of an α-adrenergic receptor agonist, an angiotensin-converting enzyme (ACE) inhibitor, an antimicrobial compound, a β-adrenergic antagonist, a carbonic anhydrase inhibitor, a nonsteroidal antiinflammatory compound, a prostaglandin, a selective cyclooxygenase-2 (COX-2) inhibitor and a steroid. The invention also provides for such compositions in a pharmaceutically acceptable carrier.

Another embodiment of the invention provides compositions comprising a therapeutically effective amount of at least one compound of the invention, that is optionally substituted with at least one NO and/or NO₂ group (i.e., nitrosylated and/or nitrosated), and at least one therapeutic agent selected from the group consisting of an α-adrenergic receptor agonist, an angiotensin-converting enzyme (ACE) inhibitor, an antimicrobial compound, a β-adrenergic antagonist, a carbonic anhydrase inhibitor, a nonsteroidal antiinflammatory compound, a prostaglandin, a selective cyclooxygenase-2 (COX-2) inhibitor and a steroid. The invention also provides for such compositions in a pharmaceutically acceptable carrier.

The invention provides methods for treating ophthalmic disorders in a patient in need thereof comprising administering to the patient a therapeutically effective amount of at least one compound of the invention, that is optionally substituted with at least one NO and/or NO₂ group (i.e., nitrosylated and/or nitrosated), and, optionally, at least one therapeutic agent, such as, for example, α-adrenergic receptor agonists, α-adrenergic receptor antagonists, angiotensin-converting enzyme (ACE) inhibitors, antimicrobial compounds, antioxidants, β-adrenergic antagonists, carbonic anhydrase inhibitors, hydralazine compounds, nonsteroidal antiinflammatory compounds (NSAIDs), prostaglandins, selective cyclooxygenase-2 (COX-2) inhibitors, and combinations of two or more thereof. The methods can optionally further comprise the administration of at least one nitric oxide donor compound. In this embodiment of the invention, the methods can involve (i) administering the nitrosated and/or nitrosylated compounds, (ii) administering the compounds, that are optionally nitrosated and/or nitrosylated, and NO donors, (iii) administering the compounds, that are optionally nitrosated and/or nitrosylated, and therapeutic agents, or (iv) administering the compounds, that are optionally nitrosated and/or nitrosylated, NO donors, and therapeutic agents. In a preferred embodiment the at least one therapeutic agent is selected from the group consisting of an α-adrenergic receptor agonist, an angiotensin-converting enzyme (ACE) inhibitor, an antimicrobial compound, a β-adrenergic antagonist, a carbonic anhydrase inhibitor, a nonsteroidal antiinflammatory compound, a prostaglandin, a selective cyclooxygenase-2 (COX-2) inhibitor, and a steroid. The compounds of the invention, nitric oxide donors, and/or therapeutic agents can be administered separately or as components of the same composition in one or more pharmaceutically acceptable carriers.

Another embodiment of the invention provides kits comprising at least one compound of the invention, that is optionally nitrosated and/or nitrosylated, and, optionally, at least one nitric oxide donor compound. The kit can further comprise at least one therapeutic agent, such as, for example, α-adrenergic receptor agonists, α-adrenergic receptor antagonists, angiotensin-converting enzyme (ACE) inhibitors, antimicrobial compounds, antioxidants, β-adrenergic antagonists, carbonic anhydrase inhibitors, hydralazine compounds, nonsteroidal antiinflammatory compounds (NSAIDs), prostaglandins, selective cyclooxygenase-2 (COX-2) inhibitors, steroids, and combinations of two or more thereof. The compounds of the invention, the nitric oxide donors and/or therapeutic agents, can be separate components in the kit or can be in the form of a composition in one or more pharmaceutically acceptable carriers.

These and other aspects of the invention are described in detail herein.

### DETAILED DESCRIPTION OF THE INVENTION

As used throughout the disclosure, the following terms, unless otherwise indicated, shall be understood to have the following meanings.

“Ophthalmic disorders” include, but are not limited to, ophthalmic infections, cataracts, glaucoma, elevated intraocular pressure, ocular pain (e.g., following corneal surgery), dry eye disorder, ocular hypertension, ocular bleeding, retinal diseases or disorders, presbyopia, macular degeneration, choroidal neovascularization (CNV), retinopathies, such as for example, diabetic retinopathy, vitreoretinopathy, and the like, retinitis, such as for example, cytomegalovirus (CMV) retinitis, uveitis, macular edema, neuropathies and the like.

“Ophthalmic infections” include, but are not limited to an inflammation of the conjunctiva (conjunctivitis), inflammation of the cornea (keratitis), corneal ulcers, and the like, caused by an organisms such as, for example, Staphylococci, Streptococci, Enterococci, Bacillus, Corynebacterium, Chlamydia, Neisseria, and the like, including important species of these genus such as, for example, Staphloccus aureus, Streptococcus viridans, Staphloccus epidermidis, Streptococcus pneumoniae, staphylococci, streptococci, enterococci, and the like.

“Therapeutic agent” includes any therapeutic agent that can be used to treat or prevent the diseases described herein. “Therapeutic agents” include, for example, α-adrenergic receptor agonists, α-adrenergic receptor antagonists, angiotensin-converting enzyme (ACE) inhibitors, antimicrobial compounds, antioxidants, β-adrenergic antagonists, carbonic anhydrase inhibitors, hydralazine compounds, nonsteroidal antiinflammatory compounds (NSAIDs), prostaglandins, selective cyclooxygenase-2 (COX-2) inhibitors, steroids, and the like. Therapeutic agent includes the pharmaceutically acceptable salts thereof, pro-drugs, and pharmaceutical derivatives thereof including, but not limited to, the corresponding nitrosated and/or nitrosylated and/or heterocyclic nitric oxide donor derivatives. Although nitric oxide donors have therapeutic activity, the term “therapeutic agent” does not include the nitric oxide donors described herein, since nitric oxide donors are separately defined.

“Prodrug” refers to a compound that is made more active in vivo.

“Antioxidant” refers to and includes any compound that can react and quench a free radical.

“Angiotensin converting enzyme (ACE) inhibitor” refers to compounds that inhibit an enzyme which catalyzes the conversion of angiotensin I to angiotensin II. ACE inhibitors include, but are not limited to, amino acids and derivatives thereof, peptides, including di- and tri-peptides, and antibodies to ACE which intervene in the renin-angiotensin system by inhibiting the activity of ACE thereby reducing or eliminating the formation of the pressor substance angiotensin II.

“α-Adrenergic receptor agonist” refers to any compound that reversibly or irreversibly activates or stimulates any α-adrenergic receptor.

“NSAID” refers to a nonsteroidal anti-inflammatory compound or a nonsteroidal anti-inflammatory drug. NSAIDs inhibit cyclooxygenase, the enzyme responsible for the biosyntheses of the prostaglandins and certain autocoid inhibitors, including inhibitors of the various isozymes of cyclooxygenase (including but not limited to cyclooxygenase-1 and -2), and as inhibitors of both cyclooxygenase and lipoxygenase.

“Cyclooxygenase-2 (COX-2) selective inhibitor” refers to a compound that selectively inhibits the cyclooxygenase-2 enzyme over the cyclooxygenase-1 enzyme. In one embodiment, the compound has a cyclooxygenase-2 IC₅₀ of less than about 2 μM and a cyclooxygenase-1 IC₅₀ of greater than about 5 μM, in the human whole blood COX-2 assay (as described in Brideau et al., Inflamm Res., 45: 68-74 (1996)) and also has a selectivity ratio of cyclooxygenase-2 inhibition over cyclooxygenase-1 inhibition of at least 10, and preferably of at least 40. In another embodiment, the compound has a cyclooxygenase-1 IC₅₀ of greater than about 1 μM, and preferably of greater than 20 μM. The compound can also inhibit the enzyme, lipoxygenase. Such selectivity may indicate an ability to reduce the incidence of common NSAID-induced side effects.

“Patient” refers to animals, preferably mammals, most preferably humans, and includes males and females, and children and adults.

“Therapeutically effective amount” refers to the amount of the compound and/or composition that is effective to achieve its intended purpose.

“Transdermal” refers to the delivery of a compound by passage through the skin and into the blood stream.

“Transmucosal” refers to delivery of a compound by passage of the compound through the mucosal tissue and into the blood stream.

“Penetration enhancement” or “permeation enhancement” refers to an increase in the permeability of the skin or mucosal tissue to a selected pharmacologically active compound such that the rate at which the compound permeates through the skin or mucosal tissue is increased.

“Carriers” or “vehicles” refers to carrier materials suitable for compound administration and include any such material known in the art such as, for example, any liquid, gel, solvent, liquid diluent, solubilizer, or the like, which is non-toxic and which does not interact with any components of the composition in a deleterious manner.

“Sustained release” refers to the release of a therapeutically active compound and/or composition such that the blood levels of the therapeutically active compound are maintained within a desirable therapeutic range over a period of time. The sustained release formulation can be prepared using any conventional method known to one skilled in the art to obtain the desired release characteristics.

“Nitric oxide adduct” or “NO adduct” refers to compounds and functional groups which, under physiological conditions, can donate, release and/or directly or indirectly transfer any of the three redox forms of nitrogen monoxide (NO⁺, NO⁻, NO.), such that the biological activity of the nitrogen monoxide species is expressed at the intended site of action.

“Nitric oxide releasing” or “nitric oxide donating” refers to methods of donating, releasing and/or directly or indirectly transferring any of the three redox forms of nitrogen monoxide (NO⁺, NO—, NO.), such that the biological activity of the nitrogen monoxide species is expressed at the intended site of action.

“Nitric oxide donor” or “NO donor” refers to compounds that donate, release and/or directly or indirectly transfer a nitrogen monoxide species, and/or stimulate the endogenous production of nitric oxide or endothelium-derived relaxing factor (EDRF) in vivo and/or elevate endogenous levels of nitric oxide or EDRF in vivo and/or are oxidized to produce nitric oxide and/or are substrates for nitric oxide synthase and/or cytochrome P450. “NO donor” also includes compounds that are precursors of L-arginine, inhibitors of the enzyme arginase and nitric oxide mediators.

“Heterocyclic nitric oxide donor” refers to a trisubstituted 5-membered ring comprising two or three nitrogen atoms and at least one oxygen atom. The heterocyclic nitric oxide donor is capable of donating and/or releasing a nitrogen monoxide species upon decomposition of the heterocyclic ring. Exemplary heterocyclic nitric oxide donors include oxatriazol-5-ones, oxatriazol-5-imines, sydnonimines, furoxans, and the like.

“Alkyl” refers to a lower alkyl group, a substituted lower alkyl group, a haloalkyl group, a hydroxyalkyl group, an alkenyl group, a substituted alkenyl group, an alkynyl group, a bridged cycloalkyl group, a cycloalkyl group or a heterocyclic ring, as defined herein. An alkyl group may also comprise one or more radical species, such as, for example a cycloalkylalkyl group or a heterocyclicalkyl group.

“Lower alkyl” refers to branched or straight chain acyclic alkyl group comprising one to about ten carbon atoms (preferably one to about eight carbon atoms, more preferably one to about six carbon atoms). Exemplary lower alkyl groups include methyl, ethyl, n-propyl, isopropyl, n-butyl, isobutyl, sec-butyl, t-butyl, pentyl, neopentyl, iso-amyl, hexyl, octyl, and the like.

“Substituted lower alkyl” refers to a lower alkyl group, as defined herein, wherein one or more of the hydrogen atoms have been replaced with one or more R¹⁰⁰ groups, wherein each R¹⁰⁰ is independently a hydroxy, an ester, an amidyl, an oxo, a carboxyl, a carboxamido, a halo, a cyano, a nitrate, a nitrite, a thionitrate, a thionitrite or an amino group, as defined herein.

“Haloalkyl” refers to a lower alkyl group, an alkenyl group, an alkynyl group, a bridged cycloalkyl group, a cycloalkyl group or a heterocyclic ring, as defined herein, to which is appended one or more halogens, as defined herein. Exemplary haloalkyl groups include trifluoromethyl, chloromethyl, 2-bromobutyl, 1-bromo-2-chloro-pentyl, and the like.

“Alkenyl” refers to a branched or straight chain C₂-C₁₀ hydrocarbon (preferably a C₂-C₈ hydrocarbon, more preferably a C₂-C₆ hydrocarbon) that can comprise one or more carbon-carbon double bonds. Exemplary alkenyl groups include propylenyl, buten-1-yl, isobutenyl, penten-1-yl, 2,2-methylbuten-1-yl, 3-methylbuten-1-yl, hexan-1-yl, hepten-1-yl, octen-1-yl, and the like.

“Lower alkenyl” refers to a branched or straight chain C₂-C₄ hydrocarbon that can comprise one or two carbon-carbon double bonds.

“Substituted alkenyl” refers to a branched or straight chain C₂-C₁₀ hydrocarbon (preferably a C₂-C₈ hydrocarbon, more preferably a C₂-C₆ hydrocarbon) which can comprise one or more carbon-carbon double bonds, wherein one or more of the hydrogen atoms have been replaced with one or more R¹⁰⁰ groups, wherein each R¹⁰⁰ is independently a hydroxy, an oxo, a carboxyl, a carboxamido, a halo, a cyano or an amino group, as defined herein.

“Alkynyl” refers to an unsaturated acyclic C₂-C₁₀ hydrocarbon (preferably a C₂-C₈ hydrocarbon, more preferably a C₂-C₆ hydrocarbon) that can comprise one or more carbon-carbon triple bonds. Exemplary alkynyl groups include ethynyl, propynyl, butyn-1-yl, butyn-2-yl, pentyl-1-yl, pentyl-2-yl, 3-methylbutyn-1-yl, hexyl-1-yl, hexyl-2-yl, hexyl-3-yl, 3,3-dimethyl-butyn-1-yl, and the like.

“Bridged cycloalkyl” refers to two or more cycloalkyl groups, heterocyclic groups, or a combination thereof fused via adjacent or non-adjacent atoms. Bridged cycloalkyl groups can be unsubstituted or substituted with one, two or three substituents independently selected from alkyl, alkoxy, amino, alkylamino, dialkylamino, hydroxy, halo, carboxyl, alkylcarboxylic acid, aryl, amidyl, ester, alkylcarboxylic ester, carboxamido, alkylcarboxamido, oxo and nitro. Exemplary bridged cycloalkyl groups include adamantyl, decahydronapthyl, quinuclidyl, 2,6-dioxabicyclo(3.3.0)octane, 7-oxabicyclo(2.2.1)heptyl, 8-azabicyclo(3,2,1)oct-2-enyl and the like.

“Cycloalkyl” refers to a saturated or unsaturated cyclic hydrocarbon comprising from about 3 to about 10 carbon atoms. Cycloalkyl groups can be unsubstituted or substituted with one, two or three substituents independently selected from alkyl, alkoxy, amino, alkylamino, dialkylamino, arylamino, diarylamino, alkylarylamino, aryl, amidyl, ester, hydroxy, halo, carboxyl, alkylcarboxylic acid, alkylcarboxylic ester, carboxamido, alkylcarboxamido, oxo, alkylsulfinyl, and nitro. Exemplary cycloalkyl groups include cyclopropyl, cyclobutyl, cyclopentyl, cyclohexyl, cyclohexenyl, cyclohepta-1,3-dienyl, and the like.

“Heterocyclic ring or group” refers to a saturated or unsaturated cyclic hydrocarbon group having about 2 to about 10 carbon atoms (preferably about 4 to about 6 carbon atoms) where 1 to about 4 carbon atoms are replaced by one or more nitrogen, oxygen and/or sulfur atoms. Sulfur maybe in the thio, sulfinyl or sulfonyl oxidation state. The heterocyclic ring or group can be fused to an aromatic hydrocarbon group. Heterocyclic groups can be unsubstituted or substituted with one, two or three substituents independently selected from alkyl, alkoxy, amino, alkylthio, aryloxy, arylthio, arylalkyl, hydroxy, oxo, thial, halo, carboxyl, carboxylic ester, alkylcarboxylic acid, alkylcarboxylic ester, aryl, arylcarboxylic acid, arylcarboxylic ester, amidyl, ester, alkylcarbonyl, arylcarbonyl, alkylsulfinyl, carboxamido, alkylcarboxamido, arylcarboxamido, sulfonic acid, sulfonic ester, sulfonamide nitrate and nitro. Exemplary heterocyclic groups include pyrrolyl, furyl, thienyl, 3-pyrrolinyl, 4,5,6-trihydro-2H-pyranyl, pyridinyl, 1,4-dihydropyridinyl, pyrazolyl, triazolyl, pyrimidinyl, pyridazinyl, oxazolyl, thiazolyl, imidazolyl, indolyl, thiophenyl, furanyl, tetrahydrofuranyl, tetrazolyl, pyrrolinyl, pyrrolindinyl, oxazolindinyl 1,3-dioxolanyl, imidazolinyl, imidazolindinyl, pyrazolinyl, pyrazolidinyl, isoxazolyl, isothiazolyl, 1,2,3-oxadiazolyl, 1,2,3-triazolyl, 1,3,4-thiadiazolyl, 2H-pyranyl, 4H-pyranyl, piperidinyl, 1,4-dioxanyl, morpholinyl, 1,4-dithianyl, thiomorpholinyl, pyrazinyl, piperazinyl, 1,3,5-triazinyl, 1,3,5-trithianyl, benzo(b)thiophenyl, benzimidazolyl, benzothiazolinyl, quinolinyl, 2,6-dioxabicyclo(3.3.0)octane, and the like.

“Heterocyclic compounds” refer to mono- and polycyclic compounds comprising at least one aryl or heterocyclic ring.

“Aryl” refers to a monocyclic, bicyclic, carbocyclic or heterocyclic ring system comprising one or two aromatic rings. Exemplary aryl groups include phenyl, pyridyl, napthyl, quinoyl, tetrahydronaphthyl, furanyl, indanyl, indenyl, indoyl, and the like. Aryl groups (including bicyclic aryl groups) can be unsubstituted or substituted with one, two or three substituents independently selected from alkyl, alkoxy, alkylthio, amino, alkylamino, dialkylamino, arylamino, diarylamino, alkylarylamino, halo, cyano, alkylsulfinyl, hydroxy, carboxyl, carboxylic ester, alkylcarboxylic acid, alkylcarboxylic ester, aryl, arylcarboxylic acid, arylcarboxylic ester, alkylcarbonyl, arylcarbonyl, amidyl, ester, carboxamido, alkylcarboxamido, carbonyl, sulfonic acid, sulfonic ester, sulfonamido and nitro. Exemplary substituted aryl groups include tetrafluorophenyl, pentafluorophenyl, sulfonamide, alkylsulfonyl, arylsulfonyl, and the like.

“Cycloalkenyl” refers to an unsaturated cyclic C₂-C₁₀ hydrocarbon (preferably a C₂-C₈ hydrocarbon, more preferably a C₂-C₆ hydrocarbon) which can comprise one or more carbon-carbon triple bonds.

“Alkylaryl” refers to an alkyl group, as defined herein, to which is appended an aryl group, as defined herein. Exemplary alkylaryl groups include benzyl, phenylethyl, hydroxybenzyl, fluorobenzyl, fluorophenylethyl, and the like.

“Arylalkyl” refers to an aryl radical, as defined herein, attached to an alkyl radical, as defined herein. Exemplary arylalkyl groups include benzyl, phenylethyl, 4-hydroxybenzyl, 3-fluorobenzyl, 2-fluorophenylethyl, and the like.

“Arylalkenyl” refers to an aryl radical, as defined herein, attached to an alkenyl radical, as defined herein. Exemplary arylalkenyl groups include styryl, propenylphenyl, and the like.

“Cycloalkylalkyl” refers to a cycloalkyl radical, as defined herein, attached to an alkyl radical, as defined herein.

“Cycloalkylalkoxy” refers to a cycloalkyl radical, as defined herein, attached to an alkoxy radical, as defined herein.

“Cycloalkylalkylthio” refers to a cycloalkyl radical, as defined herein, attached to an alkylthio radical, as defined herein.

“Heterocyclicalkyl” refers to a heterocyclic ring radical, as defined herein, attached to an alkyl radical, as defined herein.

“Arylheterocyclic ring” refers to a bi- or tricyclic ring comprised of an aryl ring, as defined herein, appended via two adjacent carbon atoms of the aryl ring to a heterocyclic ring, as defined herein. Exemplary arylheterocyclic rings include dihydroindole, 1,2,3,4-tetra-hydroquinoline, and the like.

“Alkylheterocyclic ring” refers to a heterocyclic ring radical, as defined herein, attached to an alkyl radical, as defined herein. Exemplary alkylheterocyclic rings include 2-pyridylmethyl, 1-methylpiperidin-2-one-3-methyl, and the like.

“Alkoxy” refers to R₅₀O—, wherein R₅₀ is an alkyl group, as defined herein (preferably a lower alkyl group or a haloalkyl group, as defined herein). Exemplary alkoxy groups include methoxy, ethoxy, t-butoxy, cyclopentyloxy, trifluoromethoxy, and the like.

“Aryloxy” refers to R₅₅O—, wherein R₅₅ is an aryl group, as defined herein. Exemplary arylkoxy groups include napthyloxy, quinolyloxy, isoquinolizinyloxy, and the like.

“Alkylthio” refers to R₅₀S—, wherein R₅₀ is an alkyl group, as defined herein.

“Lower alkylthio” refers to a lower alkyl group, as defined herein, appended to a thio group, as defined herein.

“Arylalkoxy” or “alkoxyaryl” refers to an alkoxy group, as defined herein, to which is appended an aryl group, as defined herein. Exemplary arylalkoxy groups include benzyloxy, phenylethoxy, chlorophenylethoxy, and the like.

“Arylalklythio” refers to an alkylthio group, as defined herein, to which is appended an aryl group, as defined herein. Exemplary arylalklythio groups include benzylthio, phenylethylthio, chlorophenylethylthio, and the like.

“Arylalklythioalkyl” refers to an arylalkylthio group, as defined herein, to which is appended an alkyl group, as defined herein. Exemplary arylalklythioalkyl groups include benzylthiomethyl, phenylethylthiomethyl, chlorophenylethylthioethyl, and the like.

“Alkylthioalkyl” refers to an alkylthio group, as defined herein, to which is appended an alkyl group, as defined herein. Exemplary alkylthioalkyl groups include allylthiomethyl, ethylthiomethyl, trifluoroethylthiomethyl, and the like.

“Alkoxyalkyl” refers to an alkoxy group, as defined herein, appended to an alkyl group, as defined herein. Exemplary alkoxyalkyl groups include methoxymethyl, methoxyethyl, isopropoxymethyl, and the like.

“Alkoxyhaloalkyl” refers to an alkoxy group, as defined herein, appended to a haloalkyl group, as defined herein. Exemplary alkoxyhaloalkyl groups include 4-methoxy-2-chlorobutyl and the like.

“Cycloalkoxy” refers to R₅₄O—, wherein R₅₄ is a cycloalkyl group or a bridged cycloalkyl group, as defined herein. Exemplary cycloalkoxy groups include cyclopropyloxy, cyclopentyloxy, cyclohexyloxy, and the like.

“Cycloalkylthio” refers to R₅₄S—, wherein R₅₄ is a cycloalkyl group or a bridged cycloalkyl group, as defined herein. Exemplary cycloalkylthio groups include cyclopropylthio, cyclopentylthio, cyclohexylthio, and the like.

“Haloalkoxy” refers to an alkoxy group, as defined herein, in which one or more of the hydrogen atoms on the alkoxy group are substituted with halogens, as defined herein. Exemplary haloalkoxy groups include 1,1,1-trichloroethoxy, 2-bromobutoxy, and the like.

“Hydroxy” refers to —OH.

“Oxy” refers to —O—

“Oxo” refers to ═O.

“Oxylate” refers to —O⁻R₇₇⁺ wherein R₇₇ is an organic or inorganic cation.

“Thiol” refers to —SH.

“Thio” refers to —S—.

“Oxime” refers to ═N—OR₈₁ wherein R₈₁ is a hydrogen, an alkyl group, an aryl group, an alkylsulfonyl group, an arylsulfonyl group, a carboxylic ester, an alkylcarbonyl group, an arylcarbonyl group, a carboxamido group, an alkoxyalkyl group or an alkoxyaryl group.

“Hydrazone refers to ═N—N(R₈₁)(R′₈₁) wherein R′₈₁ is independently selected from R₈₁, and R₈₁ is as defined herein.

“Hydrazino” refers to H₂N—N(H)—.

“Organic cation” refers to a positively charged organic ion. Exemplary organic cations include alkyl substituted ammonium cations, and the like.

“Inorganic cation” refers to a positively charged metal ion. Exemplary inorganic cations include Group I metal cations such as for example, sodium, potassium, magnesium, calcium, and the like.

“Hydroxyalkyl” refers to a hydroxy group, as defined herein, appended to an alkyl group, as defined herein.

“Nitrate” refers to —O—NO₂ i.e. oxidized nitrogen.

“Nitrite” refers to —O—NO i.e. oxidized nitrogen.

“Thionitrate” refers to —S—NO₂.

“Thionitrite” and “nitrosothiol” refer to —S—NO.

“Nitro” refers to the group —NO₂ and “nitrosated” refers to compounds that have been substituted therewith.

“Nitroso” refers to the group —NO and “nitrosylated” refers to compounds that have been substituted therewith.

“Nitrile” and “cyano” refer to —CN.

“Halogen” or “halo” refers to iodine (I), bromine (Br), chlorine (Cl), and/or fluorine (F).

“Amino” refers to —NH₂, an alkylamino group, a dialkylamino group, an arylamino group, a diarylamino group, an alkylarylamino group or a heterocyclic ring, as defined herein.

“Alkylamino” refers to R₅₀NH—, wherein R₅₀ is an alkyl group, as defined herein. Exemplary alkylamino groups include methylamino, ethylamino, butylamino, cyclohexylamino, and the like.

“Arylamino” refers to R₅₅NH—, wherein R₅₅ is an aryl group, as defined herein.

“Dialkylamino” refers to R₅₂R₅₃N—, wherein R₅₂ and R₅₃ are each independently an alkyl group, as defined herein. Exemplary dialkylamino groups include dimethylamino, diethylamino, methyl propargylamino, and the like.

“Diarylamino” refers to R₅₅R₆₀N—, wherein R₅₅ and R₆₀ are each independently an aryl group, as defined herein.

“Alkylarylamino or arylalkylamino” refers to R₅₂R₅₅N—, wherein R₅₂ is an alkyl group, as defined herein, and R₅₅ is an aryl group, as defined herein.

“Alkylarylalkylamino” refers to R₅₂R₇₉N—, wherein R₅₂ is an alkyl group, as defined herein, and R₇₉ is an arylalkyl group, as defined herein.

“Alkylcycloalkylamino” refers to R₅₂R₈₀N—, wherein R₅₂ is an alkyl group, as defined herein, and R₈₀ is a cycloalkyl group, as defined herein.

“Aminoalkyl” refers to an amino group, an alkylamino group, a dialkylamino group, an arylamino group, a diarylamino group, an alkylarylamino group or a heterocyclic ring, as defined herein, to which is appended an alkyl group, as defined herein. Exemplary aminoalkyl groups include dimethylaminopropyl, diphenylaminocyclopentyl, methylaminomethyl, and the like.

“Aminoaryl” refers to an aryl group to which is appended an alkylamino group, a arylamino group or an arylalkylamino group. Exemplary aminoaryl groups include anilino, N-methylanilino, N-benzylanilino, and the like.

“Sulfinyl” refers to —S(O)—.

“Methanthial” refers to —C(S)—.

“Thial” refers to ═S.

“Sulfonyl” refers to —S(O)₂—.

“Sulfonic acid” refers to —S(O)₂OR₇₆, wherein R₇₆ is a hydrogen, an organic cation or an inorganic cation, as defined herein.

“Alkylsulfonic acid” refers to a sulfonic acid group, as defined herein, appended to an alkyl group, as defined herein.

“Arylsulfonic acid” refers to a sulfonic acid group, as defined herein, appended to an aryl group, as defined herein

“Sulfonic ester” refers to —S(O)₂OR₅₈, wherein R₅₈ is an alkyl group, an aryl group, or an aryl heterocyclic ring, as defined herein.

“Sulfonamido” refers to —S(O)₂—N(R₅₁)(R₅₇), wherein R₅₁ and R₅₇ are each independently a hydrogen atom, an alkyl group, an aryl group or an arylheterocyclic ring, as defined herein, or R₅₁ and R₅₇ when taken together are a heterocyclic ring, a cycloalkyl group or a bridged cycloalkyl group, as defined herein.

“Alkylsulfonamido” refers to a sulfonamido group, as defined herein, appended to an alkyl group, as defined herein.

“Arylsulfonamido” refers to a sulfonamido group, as defined herein, appended to an aryl group, as defined herein.

“Alkylthio” refers to R₅₀S—, wherein R₅₀ is an alkyl group, as defined herein (preferably a lower alkyl group, as defined herein).

“Arylthio” refers to R₅₅S—, wherein R₅₅ is an aryl group, as defined herein.

“Arylalkylthio” refers to an aryl group, as defined herein, appended to an alkylthio group, as defined herein.

“Alkylsulfinyl” refers to R₅₀—S(O)—, wherein R₅₀ is an alkyl group, as defined herein.

“Alkylsulfonyl” refers to R₅₀—S(O)₂—, wherein R₅₀ is an alkyl group, as defined herein.

“Alkylsulfonyloxy” refers to R₅₀—S(O)₂—O—, wherein R₅₀ is an alkyl group, as defined herein.

“Arylsulfinyl” refers to R₅₅—S(O)—, wherein R₅₅ is an aryl group, as defined herein.

“Arylsulfonyl” refers to R₅₅—S(O)₂—, wherein R₅₅ is an aryl group, as defined herein.

“Arylsulfonyloxy” refers to R₅₅—S(O)₂—O—, wherein R₅₅ is an aryl group, as defined herein.

“Amidyl” refers to R₅₁C(O)N(R₅₇)— wherein R₅₁ and R₅₇ are each independently a hydrogen atom, an alkyl group, an aryl group or an arylheterocyclic ring, as defined herein.

“Ester” refers to R₅₁C(O)R₇₆— wherein R₅₁ is a hydrogen atom, an alkyl group, an aryl group or an arylheterocyclic ring, as defined herein and R₇₆ is oxygen or sulfur.

“Carbamoyl” refers to —O—C(O)N(R₅₁)(R₅₇), wherein R₅₁ and R₅₇ are each independently a hydrogen atom, an alkyl group, an aryl group or an arylheterocyclic ring, as defined herein, or R₅₁ and R₅₇ taken together are a heterocyclic ring, a cycloalkyl group or a bridged cycloalkyl group, as defined herein.

“Carboxyl” refers to —C(O)OR₇₆, wherein R₇₆ is a hydrogen, an organic cation or an inorganic cation, as defined herein.

“Carbonyl” refers to —C(O)—.

“Alkylcarbonyl” refers to R₅₂—C(O)—, wherein R₅₂ is an alkyl group, as defined herein.

“Arylcarbonyl” refers to R₅₅—C(O)—, wherein R₅₅ is an aryl group, as defined herein.

“Alylalkylcarbonyl” refers to R₅₅—R₅₂—C(O)—, wherein R₅₅ is an aryl group, as defined herein, and R₅₂ is an alkyl group, as defined herein.

“Alkylarylcarbonyl” refers to R₅₂—R₅₅—C(O)—, wherein R₅₅ is an aryl group, as defined herein, and R₅₂ is an alkyl group, as defined herein.

“Heterocyclicalkylcarbonyl” refer to R₇₈C(O)— wherein R₇₈ is a heterocyclicalkyl group, as defined herein.

“Carboxylic ester” refers to —C(O)OR₅₈, wherein R₅₈ is an alkyl group, an aryl group or an aryl heterocyclic ring, as defined herein.

“Alkylcarboxylic acid” and “alkylcarboxyl” refer to an alkyl group, as defined herein, appended to a carboxyl group, as defined herein.

“Alkylcarboxylic ester” refers to an alkyl group, as defined herein, appended to a carboxylic ester group, as defined herein.

“Alkyl ester” refers to an alkyl group, as defined herein, appended to an ester group, as defined herein.

“Arylcarboxylic acid” refers to an aryl group, as defined herein, appended to a carboxyl group, as defined herein.

“Arylcarboxylic ester” and “arylcarboxyl” refer to an aryl group, as defined herein, appended to a carboxylic ester group, as defined herein.

“Aryl ester” refers to an aryl group, as defined herein, appended to an ester group, as defined herein.

“Carboxamido” refers to —C(O)N(R₅₁)(R₅₇), wherein R₅₁ and R₅₇ are each independently a hydrogen atom, an alkyl group, an aryl group or an arylheterocyclic ring, as defined herein, or R₅₁ and R₅₇ when taken together are a heterocyclic ring, a cycloalkyl group or a bridged cycloalkyl group, as defined herein.

“Alkylcarboxamido” refers to an alkyl group, as defined herein, appended to a carboxamido group, as defined herein.

“Arylcarboxamido” refers to an aryl group, as defined herein, appended to a carboxamido group, as defined herein.

“Urea” refers to —N(R₅₉)—C(O)N(R₅₁)(R₅₇) wherein R₅₁, R₅₇, and R₅₉ are each independently a hydrogen atom, an alkyl group, an aryl group or an arylheterocyclic ring, as defined herein, or R₅₁ and R₅₇ taken together are a heterocyclic ring, a cycloalkyl group or a bridged cycloalkyl group, as defined herein.

“Phosphoryl” refers to —P(R₇₀)(R₇₁)(R₇₂), wherein R₇₀ is a lone pair of electrons, thial or oxo, and R₇₁ and R₇₂ are each independently a covalent bond, a hydrogen, a lower alkyl, an alkoxy, an alkylamino, a hydroxy, an oxy or an aryl, as defined herein.

“Silyl” refers to —Si(R₇₃)(R₇₄)(R₇₅), wherein R₇₃, R₇₄ and R₇₅ are each independently a covalent bond, a lower alkyl, an alkoxy, an aryl or an arylalkoxy, as defined herein.

“Organic acid” refers to compound having at least one carbon atom and one or more functional groups capable of releasing a proton to a basic group. The organic acid preferably contains a carboxyl, a sulfonic acid or a phosphoric acid moeity. Exemplary organic acids include acetic acid, benzoic acid, citric acid, camphorsulfonic acid, methanesulfonic acid, taurocholic acid, chlordronic acid, glyphosphate, medronic acid, and the like.

“Inorganic acid” refers to a compound that does not contain at least one carbon atom and is capable of releasing a proton to a basic group. Exemplary inorganic acids include hydrochloric acid, sulfuric acid, nitric acid, phosphoric acid, and the like.

“Organic base” refers to a carbon containing compound having one or more functional groups capable of accepting a proton from an acid group. The organic base preferably contains an amine group. Exemplary organic bases include triethylamine, benzyldiethylamine, dimethylethyl amine, imidazole, pyridine, pipyridine, and the like.

The compounds used in the compounds and compositions of the invention are preferably β-adrenergic antagonists and ACE inhibitors.

Suitable β-adrenergic antagonists include, but are not limited to, acebutolol, alprenolol, amosulalol, arotinolol, atenolol, befunolol, betaxolol, bevantolol, bisoprolol, bopindolol, bucindolol, bucumolol, bufetolol, bufuralol, bunitrolol, bupranolol, butofilolol, carazolol, capsinolol, carteolol, carvedilol (COREG®), celiprolol, cetamolol, cindolol, cloranolol, dilevalol, diprafenone, epanolol, ersentilide, esmolol, esprolol, hydroxalol, indenolol, labetalol, landiolol, laniolol, levobunolol, mepindolol, methylpranol, metindol, metipranolol, metrizoranolol, metoprolol, moprolol, nadolol, nadoxolol, nebivolol, nifenalol, nipradilol, oxprenolol, penbutolol, pindolol, practolol, pronethalol, propranolol, sotalol, sotalolnadolol, sulfinalol, taliprolol, talinolol, tertatolol, tilisolol, timolol, toliprolol, tomalolol, trimepranol, xamoterol, xibenolol, 2-(3-(1,1-dimethylethyl)-amino-2-hydroxypropoxy)-3-pyridenecarbonitrilHCl, 1-butylamino-3-(2,5-dichlorophenoxy)-2-propanol, 1-isopropylamino-3-(4-(2-cyclopropylmethoxyethyl) phenoxy)-2-propanol, 3-isopropylamino-1-(7-methylindan-4-yloxy)-2-butanol, 2-(3-t-butylamino-2-hydroxy-propylthio)-4-(5-carbamoyl-2-thienyl)thiazol, 7-(2-hydroxy-3-t-butylaminpropoxy)phthalide, Acc 9369, AMO-140, BIB-16S, CP-331684, Fr-172516, ISV-208, L-653328, LM-2616, SB-226552, SR-58894A, SR-59230A, TZC-5665, UK-1745, YM-430, and the like. Suitable β-adrenergic antagonists are described more fully in the literature, such as in Goodman and Gilman, The Pharmacological Basis of Therapeutics (9th Edition), McGraw-Hill, 1995; and the Merck Index on CD-ROM, 13th Edition; and on STN Express, file phar and file registry.

Suitable angiotensin-converting enzyme inhibitors (ACE inhibitors) include, but are not limited to, alacepril, benazepril (LOTENSIN®, CIBACEN®), benazeprilat, captopril, ceranapril, cilazapril, delapril, duinapril, enalapril, enalaprilat, fasidotril, fosinopril, fosinoprilat, gemopatrilat, glycopril, idrapril, imidapril, lisinopril, moexipril, moveltipril, naphthopidil, omapatrilat, pentopril, perindopril, perindoprilat, quinapril, quinaprilat, ramipril, ramiprilat, rentipril, saralasin acetate, spirapril, temocapril, trandolapril, trandolaprilat, urapidil, zofenopril, acylmercapto and mercaptoalkanoyl pralines, carboxyalkyl dipeptides, carboxyalkyl dipeptide, phosphinylalkanoyl prolines, registry no. 796406, AVE 7688, BP1.137, CHF 1514, E 4030, ER 3295, FPL-66564, MDL 100240, RL 6134, RL 6207, RL 6893, SA 760, S-5590, Z 13752A, and the like.

The contemplated compounds of the invention are described more fully in the literature, such as in Goodman and Gilman, The Pharmacological Basis of Therapeutics (9th Edition), McGraw-Hill, (1996); Merck Index on CD-ROM, 13^{th} Edition; STN Express, file phar and file registry, the disclosures of each of which are incorporated by reference herein in their entirety.

In one embodiment the compounds of the invention are β-adrenergic antagonists and ACE inhibitors, which must contain one or more of the following functionalities: a carboxylic acid group (—COOH), a hydroxyl group (—OH), a thiol group (—SH) and/or a primary or secondary amine group (—NH). The compounds of the invention are nitrosated and/or nitrosylated through one or more of these functionalities such as oxygen (hydroxyl condensation), sulfur (sulfhydryl condensation) and/or nitrogen.

In another embodiment, the invention provides nitrosated and/or nitrosylated β-adrenergic antagonists of Formula (I) and pharmaceutically acceptable salts thereof:

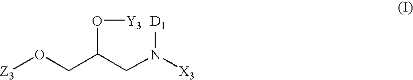

wherein:

X₃ is:
(1) —CH(CH₃)₂;
(2) —C(CH₃)₃;

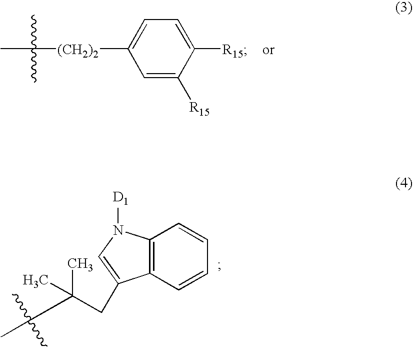

Y₃ is —C(O)—C₆H₅ or D₁;

Z₃ is:

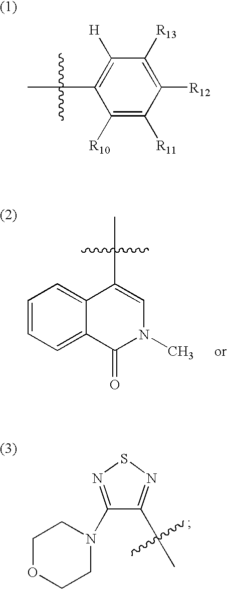

R₁₀ is:
(1) —C(O)—(CH₂)ₖ—CH₃;
(2) —O—CH₂—CH═CH₂;
(3) a hydrogen;
(4) methyl;
(5) methoxy;
(6) cyclopentyl;
(7) halo;
(8) —O—CH₂—C(O)—ND₁-CH₃;
(9) cyano;
(10) —CH₂—CH═CH₂; or

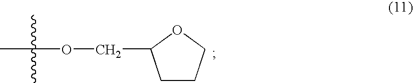

R₁₁ is a hydrogen, methyl or a halo; or

R₁₀ and R₁₁ taken together are W₄—U₄—V₄;

wherein W₄—U₄—V₄ is
(1) —CH═C(R₁₄)—ND₁-;
(2) —CH═CH—CH₂—;
(3) —CH₂—CH═CH—;
(4) —CH═CH—CH═CH—;
(5) —O—CH₂—CH(ONO₂)—CH₂—;
(6) —O—C(O)—CH═CH—;
(7) —(CH₂)₂—C(O)—ND₁-;
(8) —(CH₂)₃—C(O)—;
(9) —CH₂—CH(OD₁)-CH(OD₁)-CH₂—;
(10) —S—(CH₂)₃—;

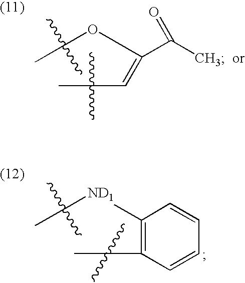

R₁₂ is:
(1) —ND₁-C(O)—(CH₂)ₖ—CH₃;
(2) —(CH₂)ₖ—C(O)—OD₁;
(3) —C(O)—(CH₂)ₖ—CH₃;
(4) halo;
(5) —ND₁-C(O)—N(C₂H₅)₂;
(6) —CH₂—C(O)—N(H)D₁;
(7) —O—C(O)—CH₃;

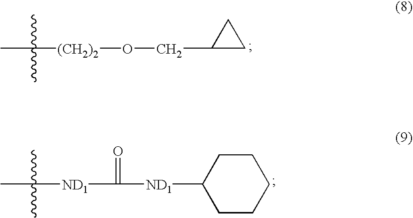

(10) —CH₂—O—(CH₂)₂—O—CH(CH₃)₂;
(11) methyl; or
(12) —(CH₂)₂—O—CH₃;

R₁₃ is a hydrogen, methyl or halo;

R₁₄ is a hydrogen or a lower alkyl;

R₁₅ at each occurrence is independently selected from —OCH₃, —OD₁, —NO₂, methyl or ND₁-S(O)₂—CH₃;

k is an integer from 0 to 4;

D₁ is a hydrogen, V₃ or K;

K is —(W₃)ₐ-E_{b}-(C(Rₑ)(R_{f}))ₚ₁-E_{c}-(C(Rₑ)(R_{f}))ₓ—(W₃)_{d}—(C(Rₑ)(R_{f}))_{y}—(W₃)ᵢ-Eⱼ-(W₃)_{g}—(C(Rₑ)(R_{f}))_{z}—U₃—V₃;

V₃ is —NO or —NO₂;

a, b, c, d, g, i and j are each independently an integer from 0 to 3;

p₁, x, y and z are each independently an integer from 0 to 10;

W₃ at each occurrence is independently —C(O)—, —C(S)—, -T₃-, —(C(Rₑ)(R_{f}))ₕ—, an alkyl group, an aryl group, a heterocyclic ring, an arylheterocyclic ring, or —(CH₂CH₂O)_{q1}—;

E at each occurrence is independently -T₃-, an alkyl group, an aryl group, —(C(Rₑ)(R_{f}))ₕ—, a heterocyclic ring, an arylheterocyclic ring, or —(CH₂CH₂O)_{q1}—;

T₃ at each occurrence is independently a covalent bond, a carbonyl, an oxygen, —S(O)ₒ— or —N(Rₐ)Rᵢ;

h is an integer form 1 to 10;

q₁ is an integer from 1 to 5;

Rₑ and R_{f} are each independently a hydrogen, an alkyl, a cycloalkoxy, a halogen, a hydroxy, an hydroxyalkyl, an alkoxyalkyl, an arylheterocyclic ring, an alkylaryl, an alkylcycloalkyl, an alkylheterocyclic ring, a cycloalkylalkyl, a cycloalkylthio, an arylalklythio, an arylalklythioalkyl, an alkylthioalkyl, a cycloalkenyl, an heterocyclicalkyl, an alkoxy, a haloalkoxy, an amino, an alkylamino, a dialkylamino, an arylamino, a diarylamino, an alkylarylamino, an alkoxyhaloalkyl, a sulfonic acid, a sulfonic ester, an alkylsulfonic acid, an arylsulfonic acid, an arylalkoxy, an alkylthio, an arylthio, a cyano, an aminoalkyl, an aminoaryl, an aryl, an arylalkyl, an alkylaryl, a carboxamido, a alkylcarboxamido, an arylcarboxamido, an amidyl, a carboxyl, a carbamoyl, an alkylcarboxylic acid, an arylcarboxylic acid, an alkylcarbonyl, an arylcarbonyl, an ester, a carboxylic ester, an alkylcarboxylic ester, an arylcarboxylic ester, a sulfonamido, an alkylsulfonamido, an arylsulfonamido, an alkylsulfonyl, an alkylsulfonyloxy, an arylsulfonyl, arylsulphonyloxy, a sulfonic ester, an alkyl ester, an aryl ester, a urea, a phosphoryl, a nitro, K or Rₑ and R_{f} taken together with the carbons to which they are attached form a carbonyl, a methanthial, a heterocyclic ring, a cycloalkyl group, an aryl group, an oxime, a hydrazone or a bridged cycloalkyl group;

U₃ at each occurrence is independently an oxygen, —S(O)ₒ— or —N(Rₐ)Rᵢ;

o is an integer from 0 to 2;

Rₐ is a lone pair of electrons, a hydrogen or an alkyl group;

Rᵢ is a hydrogen, an alkyl, an aryl, an alkylcarboxylic acid, an arylcarboxylic acid, an alkylcarboxylic ester, an arylcarboxylic ester, an alkylcarboxamido, an arylcarboxamido, an alkylaryl, an alkylsulfinyl, an alkylsulfonyl, an alkylsulfonyloxy, an arylsulfinyl, an arylsulfonyl, arylsulphonyloxy, a sulfonamido, a carboxamido, a carboxylic ester, an aminoalkyl, an aminoaryl, —CH₂—C(U₃—V₃)(Rₑ)(R_{f}), a bond to an adjacent atom creating a double bond to that atom, —(N₂O₂—)⁻.M₁⁺, wherein M₁⁺ is an organic or inorganic cation; and

with the proviso that the compounds of Formula (I) must contain at least one NO group, and/or at least one NO₂ group; wherein the at least one NO group and/or the at least one NO₂ group is linked to the β-adrenergic antagonist through an oxygen atom, a nitrogen atom or a sulfur atom.

In cases where multiple designations of variables which reside in sequence are chosen as a “covalent bond” or the integer chosen is 0, the intent is to denote a single covalent bond connecting one radical to another. For example, E₀ would denote a covalent bond, while E₂ denotes (E-E) and (C(R₄)(R₄))₂ denotes —C(R₄)(R₄)—C(R₄)(R₄)—.

In another embodiment, the invention described nitrosated and/or nitrosylated β-adrenergic antagonist of Formula (II) and pharmaceutically acceptable salts thereof:

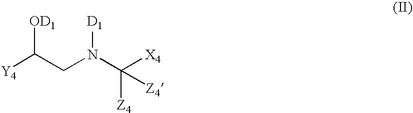

wherein:

Y₄ is:

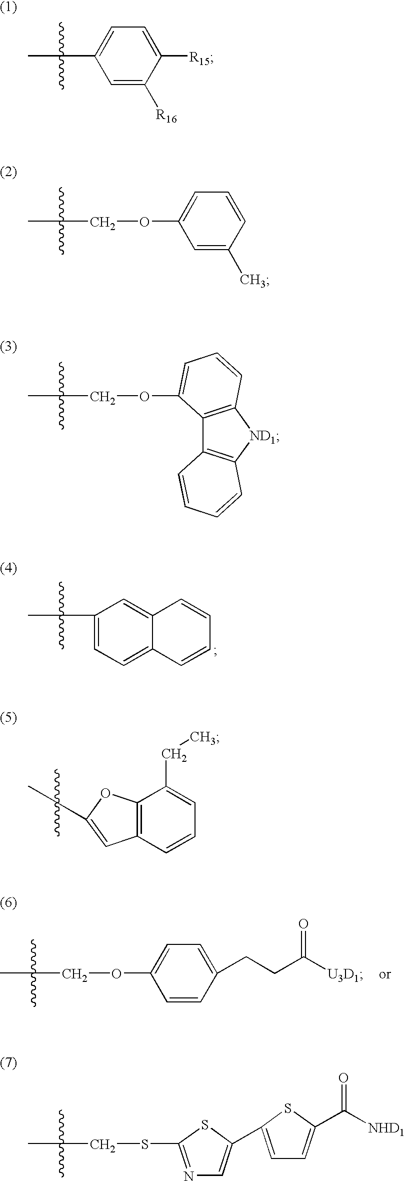

X₄ is:
(1) methyl;

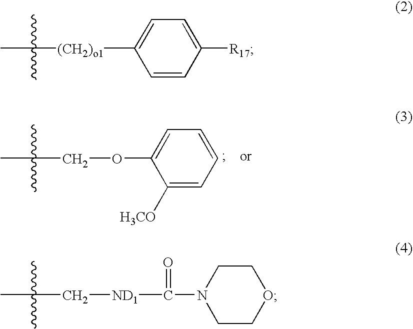

Z₄ and Z₄′ are independently selected from a methyl or a hydrogen;

R₁₆ is:
(1) hydrogen;
(2) —C(O)—N(D₁)H;
(3) —S(O)—CH₃; or
(4) —S(O)₂—N(D₁)H;

R₁₇ is a hydrogen, —OCH₃ or —NO₂;

o₁ is an integer from 0 to 2;

U₃, R₁₅ and D₁ are as defined herein; and

with the proviso that the compounds of Formula (II) must contain at least one NO group, and/or at least one NO₂ group; wherein the at least one NO group and/or the at least one NO₂ group is linked to the β-adrenergic antagonist through an oxygen atom, a nitrogen atom or a sulfur atom.

In another embodiment of the invention, the nitrosated β-adrenergic antagonist compounds of Formula (I) and (II) do not include compounds in which a —ONO₂ and/or —CH₂—ONO₂ group are directly attached to the β-adrenergic antagonist core structure (i.e. nitrosation of —OH and/or —CH₂—OH group respectively) and any of the following compounds of ACS registry number 586348-49-4, 596348-48-3, 326850-94-6, 302543-93-7, 301669-72-7, 207987-09-5, 207987-07-3, 170995-51-4, 170995-50-3, 170995-23-0, 170995-20-7, 164340-36-7, 164340-33-4, 152670-58-1, 118642-96-9, 118642-95-8, 106158-05-8, 102564-91-0, 81845-15-0, 81801-83-4, 81801-82-3, 81786-18-7, 81786-01-8, 81785-32-2, 71761-90-5, 71761-78-9, 71761-77-8, 71760-21-9 and the compounds disclosed in U.S. Pat. No. 4,288,452, U.S. Pat. No. 4,363,805, U.S. Pat. No. 4,727,085, U.S. Pat. No. 4,863,949, U.S. Pat. No. 5,502,237, U.S. Pat. No. 6,242,432, U.S. Pat. No. 6,645,965, and in WO 95/19952, WO 98/21193, WO 99/67231, WO 00/61537, WO 00/61549, WO 00/61541, WO 01/12584, WO 02/11707, WO02/053185, WO 02/053188, WO 2004/047837, WO 2004/050639 and in EP 280 951, EP 1 336 602 and in JP 05247015; the disclosures of each of which are incorporated herein in their entirety.

In another embodiment, the invention described nitrosated and/or nitrosylated angiotensin-converting enzyme (ACE) inhibitors of Formula (III) and pharmaceutically acceptable salts thereof:

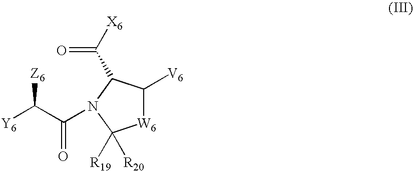

wherein:

X₆ is:
(1) —U₃D₁;
(2) —O—CH₂—CH₃; or

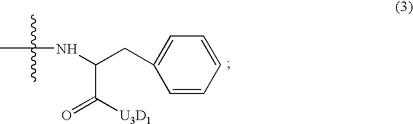

Y₆ is:
(1) —CH₂—S—R₂₁;

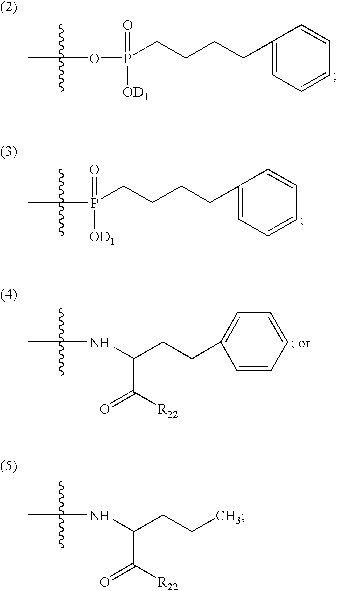

W₆ is:

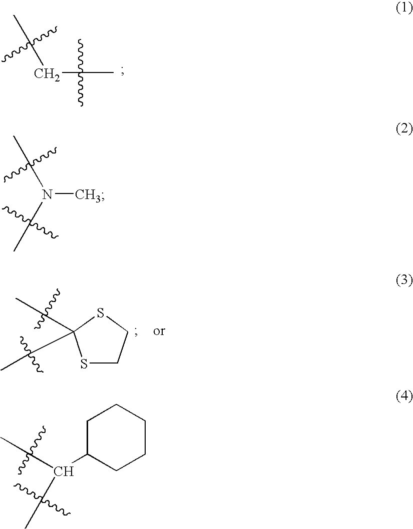

V₆ is a hydrogen;

Z₆ is:
(1) hydrogen;
(2) methyl; or
(3) —(CH₂)₄—N(H)D₁;

R₁₉ and R₂₀ are a hydrogen; or

R₁₉ and R₂₀ taken together are an oxo; or

R₂₀ and W₆ taken together are:

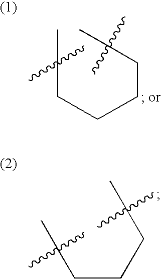

R₂₁ is:
(1) —C(O)—CH₂—CH₃;
(2) hydrogen;
(3) K; or

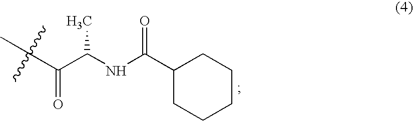

R₂₂ is —U₃D₁ or —OCH₂—CH₃;

D₁, U₃ and K are as defined herein; and

with the proviso that the compounds of Formula (III) must contain at least one NO group, and/or at least one NO₂ group; wherein the at least one NO group and/or the at least one NO₂ group is linked to the angiotensin-converting enzyme (ACE) inhibitor through an oxygen atom, a nitrogen atom or a sulfur atom.

In another embodiment, the invention described nitrosated and/or nitrosylated angiotensin-converting enzyme (ACE) inhibitors of Formula (IV) and pharmaceutically acceptable salts thereof:

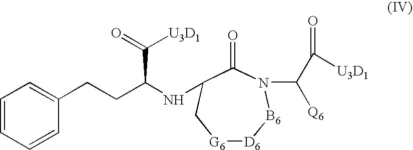

wherein:

B₆ is:

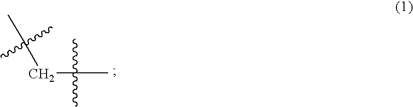

or
(2) a nitrogen;

G₆ is:

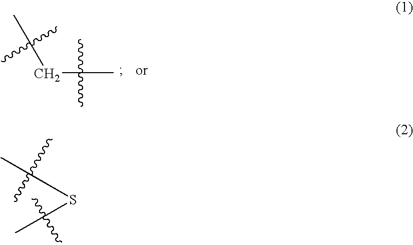

D₆ is:

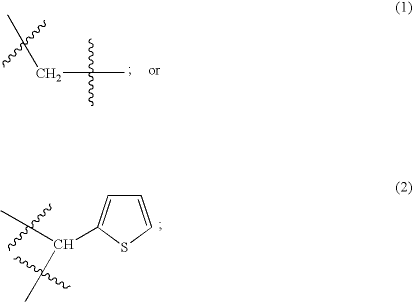

or B₆ and D₆ taken together form a phenyl ring;

Q₆ is a hydrogen; or

B₆ is a nitrogen and Q₆ is CH₂ and taken together form the ring:

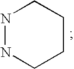

U₃ and D₁ are as defined herein; and

with the proviso that the compounds of Formula (IV) must contain at least one NO group, and/or at least one NO₂ group; wherein the at least one NO group and/or the at least one NO₂ group is linked to the angiotensin-converting enzyme (ACE) inhibitor through an oxygen atom, a nitrogen atom or a sulfur atom.

In another embodiment, the invention described nitrosated and/or nitrosylated angiotensin-converting enzyme (ACE) inhibitors of Formula (V) and pharmaceutically acceptable salts thereof:

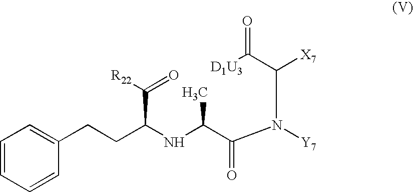

wherein:

X₇ is a hydrogen;

Y₇ is

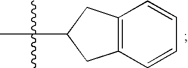

or X₇ and Y₇ taken together are:

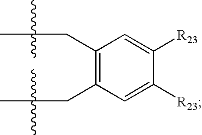

R₂₃ is a hydrogen or —OCH₃;

R₂₂, U₃ and D₁ are as defined herein; and

with the proviso that the compounds of Formula (V) must contain at least one NO group, and/or at least one NO₂ group; wherein the at least one NO group and/or the at least one NO₂ group is linked to the angiotensin-converting enzyme (ACE) inhibitor through an oxygen atom, a nitrogen atom or a sulfur atom.

In another embodiment of the invention, the nitrosated angiotensin-converting enzyme (ACE) inhibitor compounds of Formula (III), (IV) and (V) do not include compounds in which a —ONO₂ and/or —CH₂—ONO₂ group are directly attached to the angiotensin-converting enzyme (ACE) inhibitor core structure (i.e. nitrosation of —OH and/or —CH₂—OH group respectively) and any of the following compounds of ACS registry number 690655-42-6, 690655-41-5, 326850-44-6, 302543-85-7, 301669-71-6, 207987-25-5, 207987-21-1, 207987-13-1, 207987-11-9, and the compounds disclosed in U.S. Pat. No. 6,645,965, U.S. Pat. No. 6,242,432, and in WO 98/21193, WO 99/00361, WO 99/67231, WO 00/61537, WO 00/61549, WO 00/61541, WO 01/12584, WO 02/11707, WO02/053185, WO 02/053188, and in EP 1 336 602; the disclosures of each of which are incorporated herein in their entirety.

In another embodiment of the invention, the nitrosylated angiotensin-converting enzyme (ACE) inhibitor compounds of Formula (III), (IV) and (V) do not include any of the following compounds of ACS registry number 122130-63-6, and the compounds disclosed in U.S. Pat. No. 4,900,719, U.S. Pat. No. 5,002,964, U.S. Pat. No. 5,025,001, U.S. Pat. No. 5,187,183, U.S. Pat. No. 5,356,890, U.S. Pat. No. 5,536,723, and in WO 89/12627; the disclosures of each of which are incorporated herein in their entirety.

In other embodiments of the invention the compound of Formula (I) is a nitrosated acebutolol, a nitrosylated acebutolol, a nitrosated and nitrosylated acebutolol, a nitrosated alprenolol, a nitrosylated alprenolol, a nitrosated and nitrosylated alprenolol, a nitrosated atenolol, a nitrosylated atenolol, a nitrosated and nitrosylated atenolol, a nitrosated befunolol, a nitrosylated befunolol, a nitrosated and nitrosylated befunolol, a nitrosated betaxolol, a nitrosylated betaxolol, a nitrosated and nitrosylated betaxolol, a nitrosated bevantolol, a nitrosylated bevantolol, a nitrosated and nitrosylated bevantolol, a nitrosated bisoprolol, a nitrosylated bisoprolol, a nitrosated and nitrosylated bisoprolol, a nitrosated bopindolol, a nitrosylated bopindolol, a nitrosated and nitrosylated bopindolol, a nitrosated bucindolol, a nitrosylated bucindolol, a nitrosated and nitrosylated bucindolol, a nitrosated bucumolol, a nitrosylated bucumolol, a nitrosated and nitrosylated bucumolol, a nitrosated bufetolol, a nitrosylated bufetolol, a nitrosated and nitrosylated bufetolol, a nitrosated bunitrolol, a nitrosylated bunitrolol, a nitrosated and nitrosylated bunitrolol, a nitrosated bupranolol, a nitrosylated bupranolol, a nitrosated and nitrosylated bupranolol, a nitrosated butofilolol, a nitrosylated butofilolol, a nitrosated and nitrosylated butofilolol, a nitrosated carazolol, a nitrosylated carazolol, a nitrosated and nitrosylated carazolol, a nitrosated carteolol, a nitrosylated carteolol, a nitrosated and nitrosylated carteolol, a nitrosated celiprolol, a nitrosylated celiprolol, a nitrosated and nitrosylated celiprolol, a nitrosated cetamolol, a nitrosylated cetamolol, a nitrosated and nitrosylated cetamolol, a nitrosated cloranolol, a nitrosylated cloranolol, a nitrosated and nitrosylated cloranolol, a nitrosated esmolol, a nitrosylated esmolol, a nitrosated and nitrosylated esmolol, a nitrosated indenolol, a nitrosylated indenolol, a nitrosated and nitrosylated indenolol, a nitrosated levobunolol, a nitrosylated levobunolol, a nitrosated and nitrosylated levobunolol, a nitrosated mepindolol, a nitrosylated mepindolol, a nitrosated and nitrosylated mepindolol, a nitrosated metipranolol, a nitrosylated metipranolol, a nitrosated and nitrosylated metipranolol, a nitrosated metoprolol, a nitrosylated metoprolol, a nitrosated and nitrosylated metoprolol, a nitrosated moprolol, a nitrosylated moprolol, a nitrosated and nitrosylated moprolol, a nitrosated nadolol, a nitrosylated nadolol, a nitrosated and nitrosylated nadolol, a nitrosated nipradilol, a nitrosylated nipradilol, a nitrosated and nitrosylated nipradilol, a nitrosated oxprenolol, a nitrosylated oxprenolol, a nitrosated and nitrosylated oxprenolol, a nitrosated penbutolol, a nitrosylated penbutolol, a nitrosated and nitrosylated penbutolol, a nitrosated pindolol, a nitrosylated pindolol, a nitrosated and nitrosylated pindolol, a nitrosated practolol, a nitrosylated practolol, a nitrosated and nitrosylated practolol, a nitrosated propranolol, a nitrosylated propranolol, a nitrosated and nitrosylated propranolol, a nitrosated talinolol, a nitrosylated talinolol, a nitrosated and nitrosylated talinolol, a nitrosated tertatolol, a nitrosylated tertatolol, a nitrosated and nitrosylated tertatolol, a nitrosated tilisolol, a nitrosylated tilisolol, a nitrosated and nitrosylated tilisolol, a nitrosated timolol, a nitrosylated timolol, a nitrosated and nitrosylated timolol, a nitrosated toliprolol, a nitrosylated toliprolol, a nitrosated and nitrosylated toliprolol, a nitrosated xibenolol, a nitrosylated xibenolol, a nitrosated and nitrosylated xibenolol; the compound of Formula (II) is a nitrosated amosulalol, a nitrosylated amosulalol, a nitrosated and nitrosylated amosulalol, a nitrosated arotinolol, a nitrosylated arotinolol, a nitrosated and nitrosylated arotinolol, a nitrosated bufuralol, a nitrosylated bufuralol, a nitrosated and nitrosylated bufuralol, a nitrosated carvedilol, a nitrosylated carvedilol, a nitrosated and nitrosylated carvedilol, a nitrosated dilevalol, a nitrosylated dilevalol, a nitrosated and nitrosylated dilevalol, a nitrosated labetalol, a nitrosylated labetalol, a nitrosated and nitrosylated labetalol, a nitrosated landiolol, a nitrosylated landiolol, a nitrosated and nitrosylated landiolol, a nitrosated nifenalol, a nitrosylated nifenalol, a nitrosated and nitrosylated nifenalol, a nitrosated pronethalol, a nitrosylated pronethalol, a nitrosated and nitrosylated pronethalol, a nitrosated sotalol, a nitrosylated sotalol, a nitrosated and nitrosylated sotalol, a nitrosated sulfinalol, a nitrosylated sulfinalol, a nitrosated and nitrosylated sulfinalol; the compound of Formula (III) is a nitrosated alacepril, a nitrosylated alacepril, a nitrosated and nitrosylated alacepril, a nitrosated captopril, a nitrosylated captopril, a nitrosated and nitrosylated captopril, a nitrosated ceronapril, a nitrosylated ceronapril, a nitrosated and nitrosylated ceronapril, a nitrosated enalapril, a nitrosylated enalapril, a nitrosated and nitrosylated enalapril, a nitrosated enalaprilat, a nitrosylated enalaprilat, a nitrosated and nitrosylated enalaprilat, a nitrosated fosinopril, a nitrosylated fosinopril, a nitrosated and nitrosylated fosinopril, a nitrosated imidapril, a nitrosylated imidapril, a nitrosated and nitrosylated imidapril, a nitrosated lisinopril, a nitrosylated lisinopril, a nitrosated and nitrosylated lisinopril, a nitrosated moveltipril, a nitrosylated moveltipril, a nitrosated and nitrosylated moveltipril, a nitrosated perindopril, a nitrosylated perindopril, a nitrosated and nitrosylated perindopril, a nitrosated ramipril, a nitrosylated ramipril, a nitrosated and nitrosylated ramipril, a nitrosated spirapril, a nitrosylated spirapril, a nitrosated and nitrosylated spirapril, a nitrosated trandolapril, a nitrosylated trandolapril, a nitrosated and nitrosylated trandolapril; the compound of Formula (IV) is a nitrosated benazepril, a nitrosylated benazepril, a nitrosated and nitrosylated benazepril, a nitrosated cilazapril, a nitrosylated cilazapril, a nitrosated and nitrosylated cilazapril, a nitrosated temocapril, a nitrosylated temocapril, a nitrosated and nitrosylated temocapril; the compound of Formula (V) is a nitrosated delapril, a nitrosylated delapril, a nitrosated and nitrosylated delapril, a nitrosated moexipril, a nitrosylated moexipril, a nitrosated and nitrosylated moexipril, a nitrosated quinapril, a nitrosylated quinapril, a nitrosated and nitrosylated quinapril, and pharmaceutically acceptable salts thereof.

In one embodiment of the invention for the nitrosated compounds of Formula (I), (II), (III), (IV) or (V) and pharmaceutically acceptable salts thereof, K is:

(1) —Y—(CR₄R₄′)ₚ-T-(CR₄R₄′)ₚ—ONO₂;

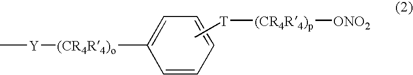

wherein T is ortho, meta or para;

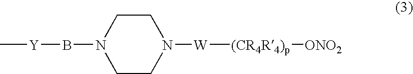

(4) —Y—(CR₄R₄′)ₚ—V—B-T-(CR₄R₄′)ₚ—ONO₂;

(5) —Y—(CR₄R₄′)ₚ-T-C(O)—(CR₄R₄′)_{q}—(CH₂)—ONO₂;

(6) —Y—(CR₄R₄′)ₚ—C(Z)-(CH₂)_{q}-T-(CR₄R₄′)_{q}—(CH₂)—ONO₂;

(7) —Y—(CR₄R₄′)ₚ-T-(CH₂)_{q}V—(CR₄R₄′)_{q}—(CH₂)—ONO₂;

(8) —Y—(CR₄R₄′)ₚ—V—(CH₂)_{q}—V—(CR₄R₄′)_{q}—(CH₂)—ONO₂;

(9) —Y—(CR₄R₄′)ₒ—(W)_{q}—(CR₄R₄′)ₒ—(CH₂)—ONO₂;

(10) —NRⱼ—O—(CH₂)ₒ—V—(CR₄R₄′)_{q}—(CH₂)—ONO₂;

(11) —NRⱼ—O—(CH₂)ₒ—(W)_{q}—(CR₄R₄′)_{q}—(CH₂)—ONO₂;

(12) —O—NRⱼ—(CH₂)ₒ—(W)_{q}—(CR₄R₄′)_{q}—(CH₂)—ONO₂;

(13) —Y—(CH₂)ₒ—(W)_{q}—(CH₂)ₒ—V—(CR₄R₄′)ₒ-Q′-(CR₄R₄′)ₒ—(CH₂)—ONO₂;

(14) —Y—(CR₄R₄′)ₚ—V—(CH₂)ₒ—(W)_{q}—(CR₄R₄′)_{q}—(CH₂)—ONO₂;

(15) —O—NRⱼ—(CH₂)ₒ—V—(CR₄R₄′)_{q}—(CH₂)—ONO₂;

(16) —Y—(CR₄R₄′)ₒ-Q′-(CR₄R₄′)ₒ—V—(CR₄R₄′)ₒ—(CH₂)—ONO₂;

(17) —Y—(CR₄R₄′)ₒ-Q′-(CR₄R₄′)ₒ—(W)_{q}—(CR₄R₄′)ₙ—(CH₂)—ONO₂;

(18) —Y—(CR₄R₄′)ₚ-T-(CR₄R₄′)ₚ-Q′-(CR₄R₄′)ₒ—(CH₂)—ONO₂;

(19) —Y—(CR₄R₄′)_{q}—C(Z)-(CR₄R₄′)ₒ—(CH₂)—ONO₂;

(20) —Y—(CR₄R₄′)ₚ-Q′-(CR₄R₄′)ₒ—(CH₂)—ONO₂;

(21) —Y—(CR₄R₄′)_{q}—P(O)MM′;

(22) —Y—(CR₄R₄′)ₒ-Q′-(CR₄R₄′)ₙ—(CH₂)—ONO₂;

(23) —Y—(CR₄R₄′)ₒ-Q′-(CR₄R₄′)ₒ-T-(CR₄R₄′)ₒ—(CH₂)—ONO₂;

(24) —Y—(CR₄R₄′)_{q}—(W)_{q}—(CR₄R₄′)ₒ-Q′-(CR₄R₄′)—(CH₂)—ONO₂;

(25) —Y—(CR₄R₄′)_{q}—V—(CR₄R₄′)ₒ-Q′-(CR₄R₄′)ₒ—(CH₂)—ONO₂;

(26) —Y—(CR₄R₄′)ₚ-(T)ₒ-(W)_{q}—(CR₄R₄′)ₒ—(CH₂)—ONO₂;

(27) —Y—(CR₄R₄′)ₚ—(W)_{q}-(T)ₒ-(CR₄R₄′)ₒ—(CH₂)—ONO₂;

(28) —Y—(CR₄R₄′)_{q}—C(Z)-V—(CR₄R₄′)_{q}—(CH₂)—ONO₂;

(29) —Y—(CR₄R₄′)ₒ—C(R₄)(ONO₂)—(CR₄R₄′)_{q}-(T)ₒ-(W)_{q}-(T)ₒ-(CR₄R₄′)ₒ—R₅;

(30) —Y—(CR₄R₄′)ₒ—V—(CR₄R₄′)ₒ-Q′-(CR₄R₄′)ₒ—(CH₂)—ONO₂;

(31) —Y—(CR₄R₄′)_{q}—C(Z)-Q′-(CR₄R₄′)ₒ—(CH₂)—ONO₂;

(32) —Y—(CR₄R₄′)ₚ—V—(CR₄R₄′)ₚ—(CH₂)—ONO₂;

(33) —Y—(CR₄R₄′)ₚ—V—(CH₂)_{q}-(T)ₒ-(CR₄R₄′)_{q}—(CH₂)—ONO₂;

(34) —Y—(CR₄R₄′)ₚ-(T)ₒ-Q′-(T)ₒ-(CR₄R₄′)_{q}—(CH₂)—ONO₂;

(35) —Y—(CR₄R₄′)_{q}—C(Z)-(CR₄R₄′)_{q}—V—(CR₄R₄′)ₒ-Q′-(CR₄R₄′)ₒ—(CH₂)—ONO₂;

(36) —Y—(CR₄R₄′)_{q}—C(Z)-(CR₄R₄′)_{q}—(W)_{q}—(CR₄R₄′)ₒ-Q′-(CR₄R₄′)ₒ—(CH₂)—ONO₂;

(37) —NRⱼ—O—(CH₂)ₒ—V—(CR₄R₄′)ₒ-Q′-(CH₂)—ONO₂;

(38) —NRⱼ—O—(CH₂)ₒ—(W)_{q}—(CR₄R₄′)ₒ-Q′-(CH₂)—ONO₂;

(39) —O—NRⱼ—(CH₂)ₒ—(W)_{q}—(Cr₄R₄′)ₒ-Q′-(CH₂)—ONO₂;

(40) —O—NRⱼ—(CH₂)ₒ—V—(CR₄R₄′)ₒ-Q′-(CH₂)—ONO₂;

(41) —NRⱼ—NRⱼ—(CR₄R₄′)ₚ—(W)_{q}-(T)ₒ(CR₄R₄′)ₒ—(CH₂)—ONO₂;

(42) —Y—(CR₄R₄′)ₒ-Q′-(CR₄R₄′)ₒ—ONO₂; or

(43) —Y—(CR₄R₄′)ₒ—V—(CR₄R₄′)ₒ-Q-(CR₄R₄′)ₒ—ONO₂;

R₄ and R₄′ at each occurrence are independently a hydrogen, lower alkyl group, —OH, —CH₂OH, —ONO₂, —NO₂ or —CH₂ONO₂; or R₄ and R₄′ taken together with the carbon atom to which they are attached are a cycloalkyl group or a heterocyclic ring;

V is —C(O)-T-, -T-C(O)—, -T-C(O)-T or T-C(O)—C(O)-T;

W is a covalent bond or a carbonyl group;

T at each occurrence is independently an oxygen, (S(O)ₒ)ₒ or NRⱼ;

Rⱼ is a hydrogen, an alkyl group, an aryl group, a heterocyclic ring, an alkylcarbonyl group, an alkylaryl group, an alkylsulfinyl group, an alkylsulfonyl group, an arylsulfinyl group, an arylsulfonyl group, a sulfonamido group, a N-alkylsulfonamido group, a N,N-diarylsulfonamido group, a N-arylsulfonamido group, a N-alkyl-N-arylsulfonamido group, a carboxamido group or a hydroxyl group;

p at each occurrence is independently an integer from 1 to 6;

q at each occurrence is independently an integer from 1 to 3;

o at each occurrence is independently an integer from 0 to 2;

Y is independently a covalent bond, a carbonyl, an oxygen, —S(O)ₒ— or —NRⱼ;

B is either phenyl or (CH₂)ₒ;

Q′ is a cycloalkyl group, a heterocyclic ring or an aryl group;

Z is (═O), (═N—OR₅), (═N—NR₅R′₅) or (═CR₅R′₅);

M and M′ are each independently —O⁻H₃N⁺—(CR₄R′₄)_{q}—CH₂ONO₂ or -T-(CR₄R′₄)ₒ—CH₂ONO₂; and

R₅ and R₅′ at each occurrence are independently a hydrogen, a hydroxyl group, an alkyl group, an aryl group, an alkylsulfonyl group, an arylsulfonyl group, a carboxylic ester, an alkylcarbonyl group, an arylcarbonyl group, a carboxamido group, an alkoxyalkyl group, an alkoxyaryl group, a cycloalkyl group or a heterocyclic ring.

In one embodiment of the invention for the nitrosated compounds of Formula (I), (II), (III), (IV) or (V) and pharmaceutically acceptable salts thereof, K is:

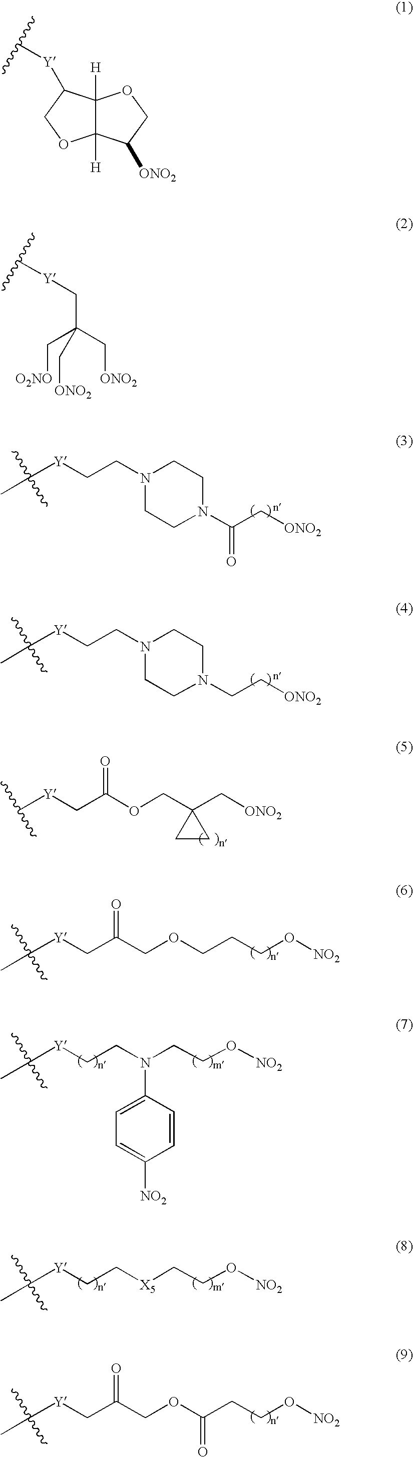

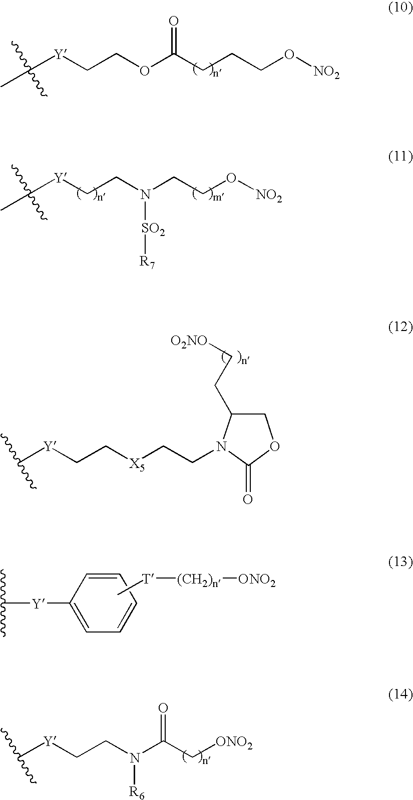

wherein T′ maybe ortho, meta or para

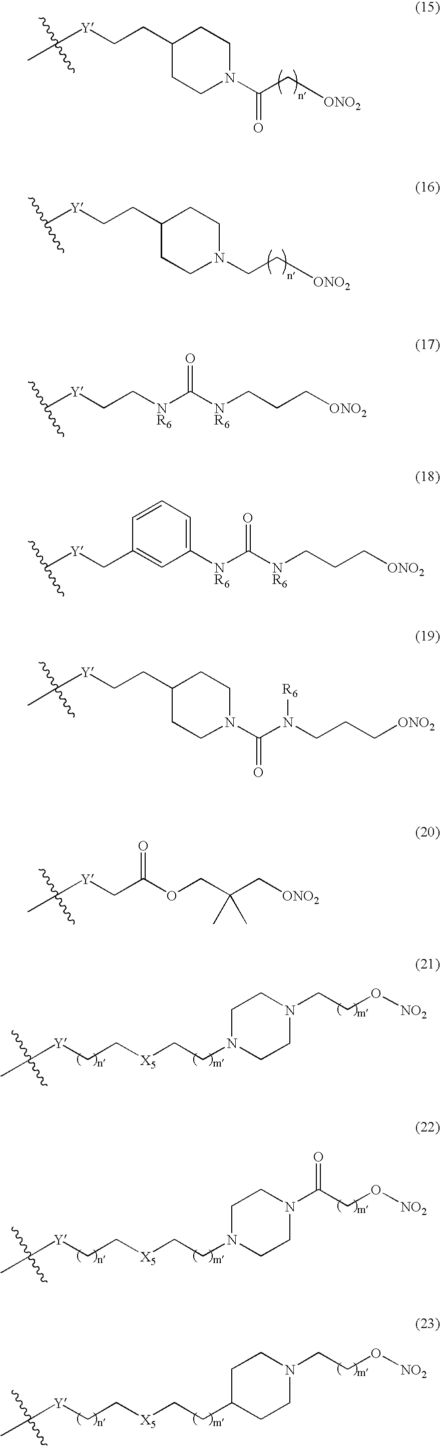

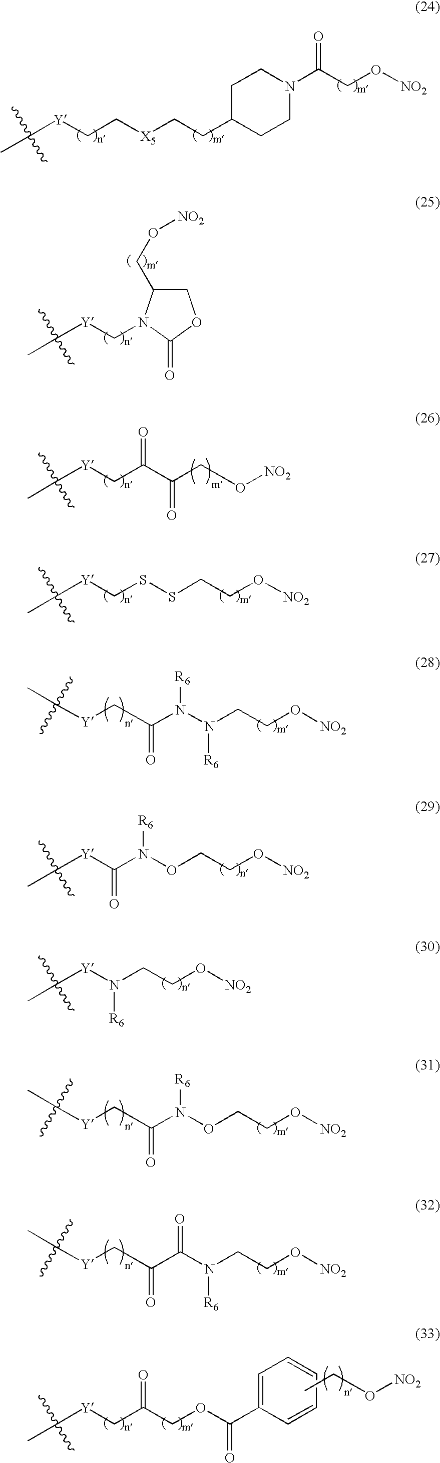

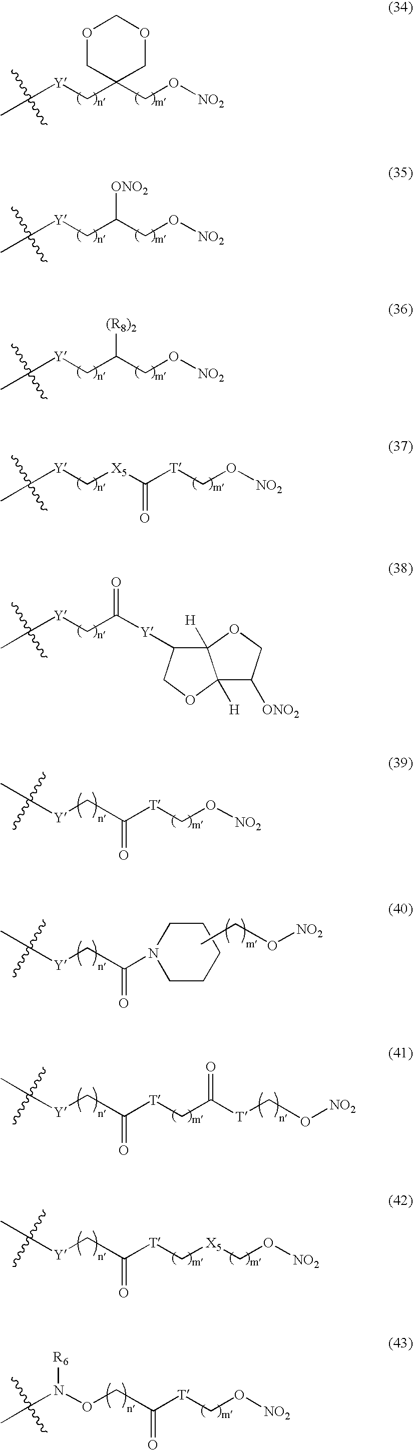

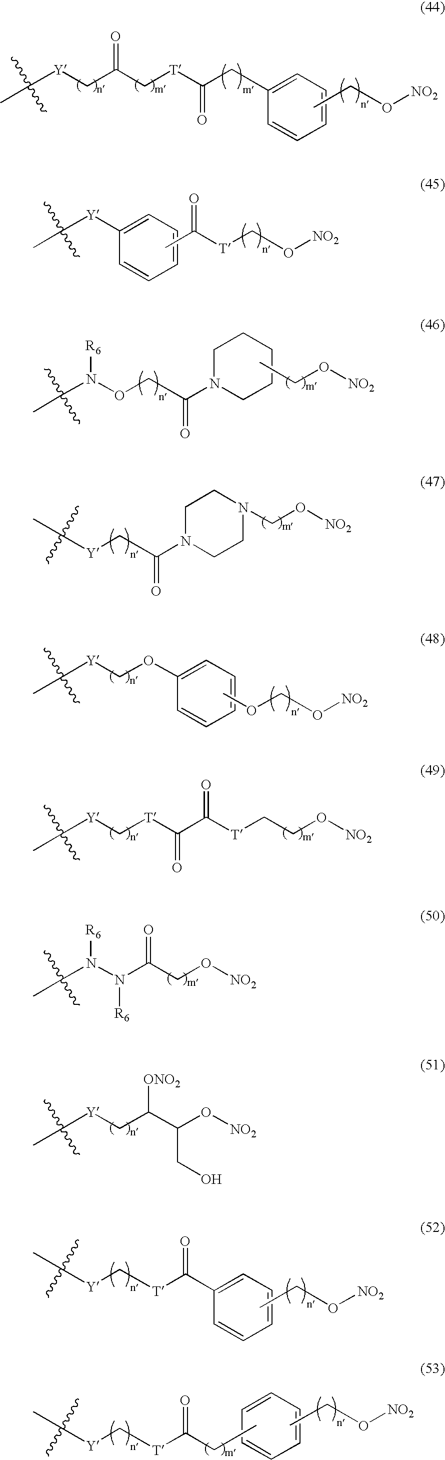

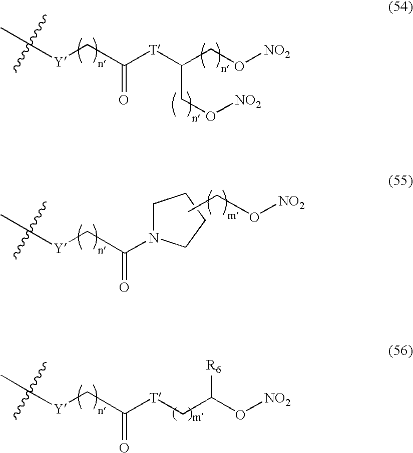

wherein:

Y′ a covalent bond, a carbonyl, an oxygen, —S(O)ₒ— or —NR₆;
T′ is oxygen, sulfur or NR₆;
X₅ is oxygen, (S(O)ₒ)ₒ or NR₆;

R₆ is a hydrogen, a lower alkyl group, an aryl group;

R₇ is a lower alkyl group or an aryl group;

R₈ at each occurrence is independently is a hydrogen, a hydroxyl group, a lower alkyl group, an aryl group, —NO₂, —CH₂—ONO₂ or —CH₂—OH;

n′ and m′ are each independently an integer from 0 to 10; and

o is an integer from 0 to 2.

In other embodiments of the invention, the β-adrenergic antagonists of Formula (I) is a nitrosated atenolol of Formula (VI), a nitrosated bisoprolol of Formula (VII), a nitrosated metoprolol of Formula (VIII), a nitrosated propranolol of Formula (IX), a nitrosated timolol of Formula (X), a nitrosated betaxolol of Formula (XI); the β-adrenergic antagonist of Formula (II) is a nitrosated carvedilol of Formula (XII), and the nitrosated angiotensin-converting enzyme (ACE) inhibitor of Formula (III) is a nitrosated captopril of Formula (XIII), a nitrosated enalapril of Formula (XIV), a nitrosated fosinopril of Formula (XV), a nitrosated lisinopril of Formula (XVI), a nitrosated ramipril of Formula (XVII) or Formula (XVIII), a nitrosated trandolaprilat of Formula (XIX); a nitrosated trandolapril of Formula (XIX) or Formula (XX), the nitrosated angiotensin-converting enzyme inhibitor of Formula (IV) is a nitrosated benazepril of Formula (XXI) or Formula (XXII); the nitrosated angiotensin-converting enzyme inhibitor of Formula (V) is a nitrosated moexipril of Formula (XXIII) or Formula (XXIV), a nitrosated quinapril of Formula (XXV) or Formula (XXVI), or a pharmaceutically acceptable salt thereof,

wherein the compound of Formula (VI) is:

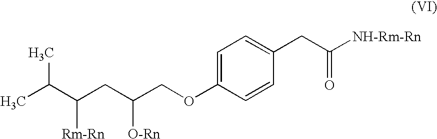

and the compound of Formula (VII) is:

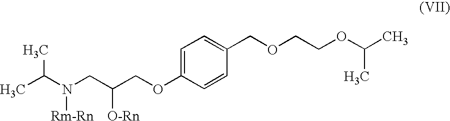

and the compound of Formula (VIII) is:

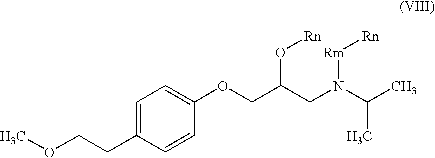

and the compound of Formula (IX) is:

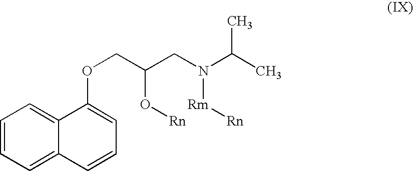

and the compound of Formula (X) is:

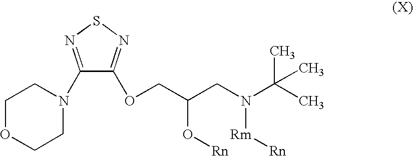

and the compound of Formula (XI) is:

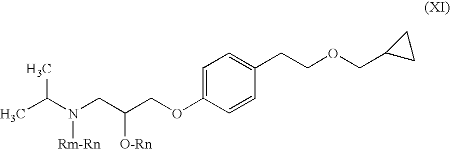

and the compound of Formula (XII) is:

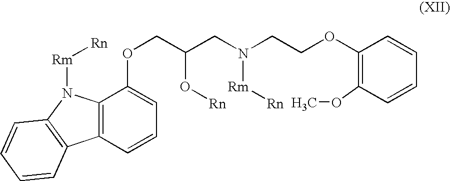

and the compound of Formula (XIII) is:

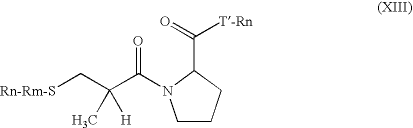

and the compound of Formula (XIV) is:

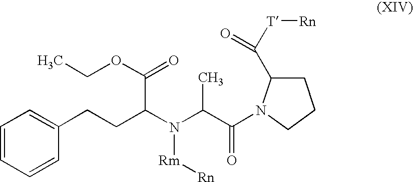

and the compound of Formula (XV) is:

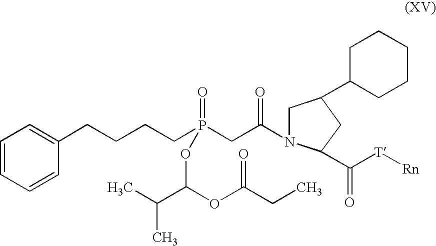

and the compound of Formula (XVI) is:

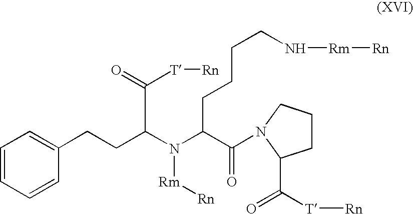

and the compound of Formula (XVI) is:

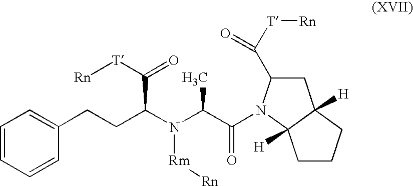

and the compound of Formula (XVII) is:

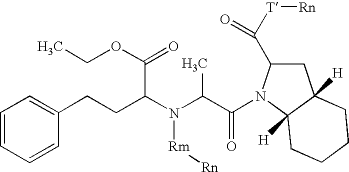

and the compound of Formula (XIX) is:

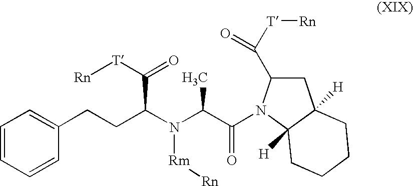

and the compound of Formula (XX) is:

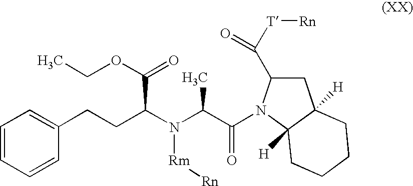

and the compound of Formula (XXI) is:

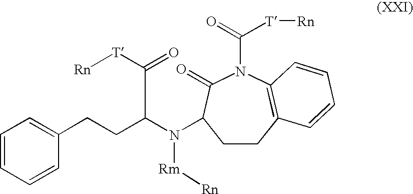

and the compound of Formula (XXII) is:

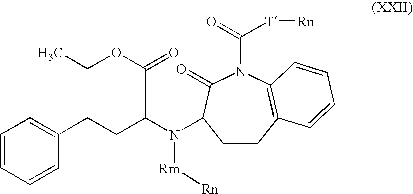

and the compound of Formula (XXM) is:

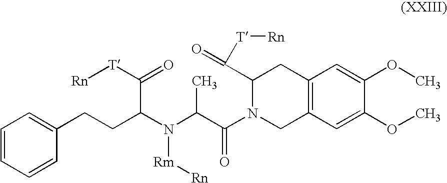

and the compound of Formula (XXIV) is:

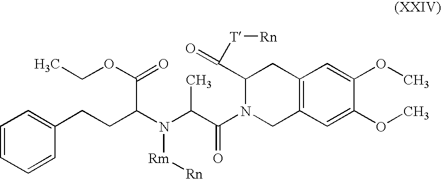

and the compound of Formula (XXV) is:

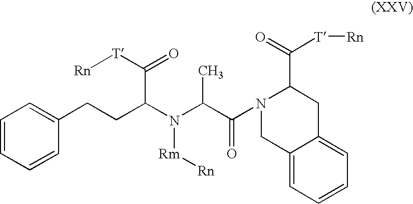

and the compound of Formula (XXVI) is:

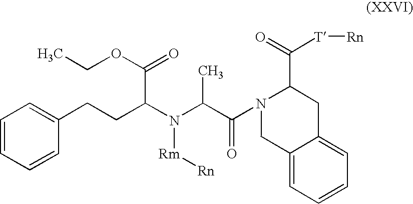

wherein

T′ is oxygen, sulfur or NR₆;

R₆ is a hydrogen, a lower alkyl group, an aryl group;

Rₘ-Rₙ taken together can be a hydrogen atom; or

Rₘ is:
(i) —C—(O)—;
(ii) —C—(O)—NR₆;
(iii) —C(O)—O—;
(iv) —C(O)—S;
(v) —CH₂—O—; or
(vi) —CH(CH₃)—O—;

Rₙ is:

a hydrogen or

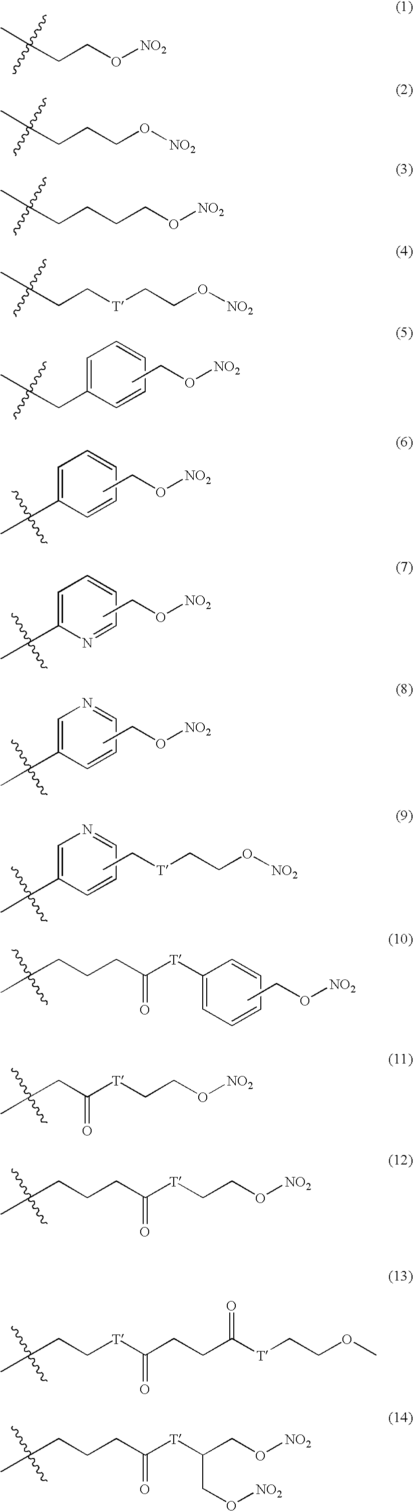

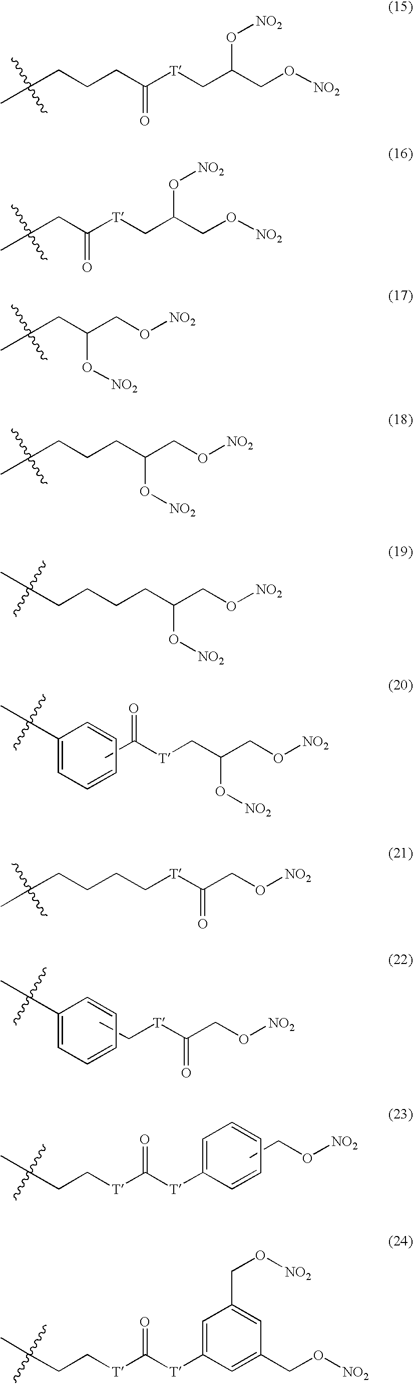

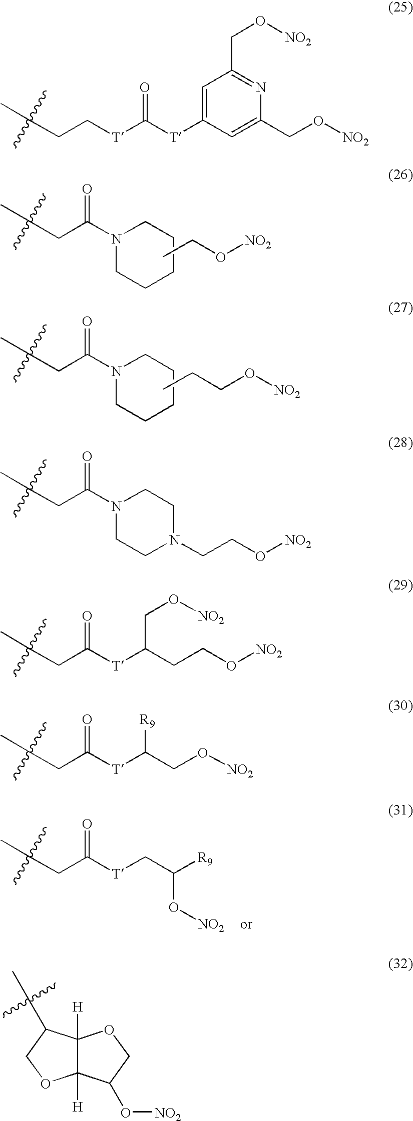

wherein:

R₉ is a lower alkyl group;

T′ is oxygen, sulfur or NR₆;

R₆ is a hydrogen, a lower alkyl group, an aryl group; and

with the proviso that the compounds of Formula (IV) to Formula (XXVI) must contain at least one —NO₂ group.

In one embodiment the nitrosated ACE inhibitors of Formula (III) are the compounds of Formula (XXVII), (XXVIII) and (XXIX), or pharmaceutically acceptable salts thereof:

wherein the compound of Formula (XXVII), ethyl (2S)-2-(((1S)-2-((2S)-2-(((2S,6R)-6-(nitrooxy)-4,8-dioxabicyclo(3.3.0)oct-2-yl)oxycarbonyl)pyrrolidinyl)-1-methyl-2-oxoethyl)amino)-4-phenylbutanoate is:

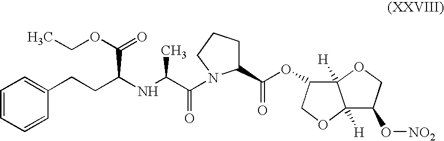

wherein the compound of Formula (XXIX), (2S)-1-((2S)-2-(((1S)-1-(((2S,6R)-6-(nitrooxy)-4,8-dioxabicyclo(3.3.0)oct-2-yl)oxycarbonyl)-3-phenylpropyl)amino)propanoyl)pyrrolidine-2-carboxylic acid is:

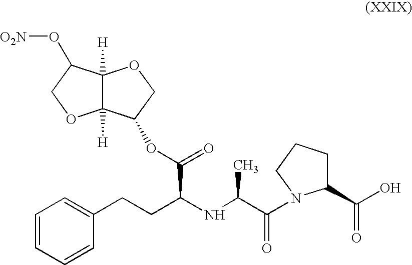

wherein the compound of Formula (XXX), (2S,6R)-6-(nitrooxy)-4,8-dioxabicyclo(3.3.0)oct-2-yl (2S)-2-(((1S)-2-((2S)-2-(((2S,6R)-6-(nitrooxy)-4,8-dioxabicyclo(3.3.0)oct-2-yl)oxycarbonyl)pyrrolidinyl)-1-methyl-2-oxoethyl)amino)-4-phenylbutanoate is:

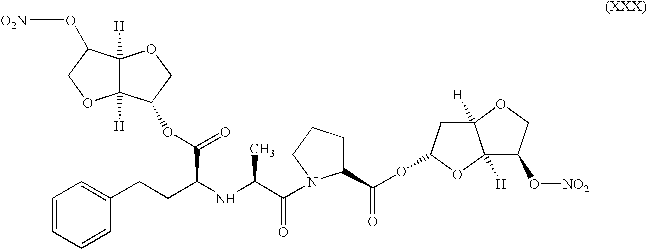

In another embodiment, the invention describes nitrosated compounds of the invention and pharmaceutically acceptable salts thereof. In one embodiment, the pharmaceutically acceptable salts do not include the nitrate salt.

Compounds of the invention that have one or more asymmetric carbon atoms may exist as the optically pure enantiomers, pure diastereomers, mixtures of enantiomers, mixtures of diastereomers, racemic mixtures of enantiomers, diastereomeric racemates or mixtures of diastereomeric racemates. It is to be understood that the invention anticipates and includes within its scope all such isomers and mixtures thereof.

Another embodiment of the invention describes the metabolites of the nitrosated and/or nitrosylated compounds and pharmaceutically acceptable salts thereof. These metabolites, include but are not limited to, the non-nitrosated and/or nitrosylated derivatives, degradation products, hydrolysis products, and the like, of the nitrosated and/or nitrosylated compounds and pharmaceutically acceptable salts thereof.

Another embodiment of the invention provides processes for making the novel compounds of the invention and to the intermediates useful in such processes. The reactions are performed in solvents appropriate to the reagents and materials used are suitable for the transformations being effected. It is understood by one skilled in the art of organic synthesis that the functionality present in the molecule must be consistent with the chemical transformation proposed. This will, on occasion, necessitate judgment by the routineer as to the order of synthetic steps, protecting groups required, and deprotection conditions. Substituents on the starting materials may be incompatible with some of the reaction conditions required in some of the methods described, but alternative methods and substituents compatible with the reaction conditions will be readily apparent to one skilled in the art. The use of sulfur and oxygen protecting groups is well known for protecting thiol and alcohol groups against undesirable reactions during a synthetic procedure and many such protecting groups are known and described by, for example, Greene and Wuts, Protective Groups in Organic Synthesis, Third Edition, John Wiley & Sons, New York (1999).

The chemical reactions described herein are generally disclosed in terms of their broadest application to the preparation of the compounds of this invention. Occasionally, the reactions may not be applicable as described to each compound included within the disclosed scope. The compounds for which this occurs will be readily recognized by one skilled in the art. In all such cases, either the reactions can be successfully performed by conventional modifications known to one skilled in the art, e.g., by appropriate protection of interfering groups, by changing to alternative conventional reagents, by routine modification of reaction conditions, and the like, or other reactions disclosed herein or otherwise conventional, will be applicable to the preparation of the corresponding compounds of this invention. In all preparative methods, all starting materials are known or readily prepared from known starting materials.

The compounds of Formulas (I) to (XXX) can be synthesized by one skilled in the art using conventional methods. Some of the parent compounds (i.e. non-nitrosated and/or non-nitrosylated angiotensin-converting enzyme (ACE) inhibitors and β-adrenergic antagonists) are commercially available or their synthesis has been reported in the scientific literature. The compounds are nitrosated and/or nitrosylated through one or more sites such as oxygen, sulfur and/or nitrogen using conventional methods known to one skilled in the art. Known methods for nitrosating and/or nitrosylating compounds are described in U.S. Pat. Nos. 5,380,758, 5,859,053, 5,703,073 and 6,297,260; and in WO 94/03421, WO 94/04484, WO 94/12463, WO 95/09831, WO 95/19952, WO 95/30641, WO 97/27749, WO 98/09948, WO 98/19672, WO 98/21193, WO 00/51988, WO 00/61604, WO 00/72838, WO 01/00563, WO 01/04082, WO 01/10814, WO 01/12584, WO 01/45703, WO 00/61541, WO 00/61537, WO 02/11707, WO 02/30866 and in Oae et al, Org. Prep. Proc. Int., 15(3):165-198 (1983), the disclosures of each of which are incorporated by reference herein in their entirety. The methods of nitrosating and/or nitrosylating the compounds described in these references can be applied by one skilled in the art to produce any of the nitrosated and/or nitrosylated compounds described herein. The nitrosated and/or nitrosylated compounds of the invention donate, transfer or release a biologically active form of nitrogen monoxide (i.e., nitric oxide).

Compounds contemplated for use in the invention, e.g., compounds that are nitrosated and/or nitrosylated, through one or more sites such as oxygen (hydroxyl condensation), sulfur (sulfhydryl condensation) and/or nitrogen, are, optionally, used in combination with nitric oxide and compounds that release nitric oxide or otherwise directly or indirectly deliver or transfer a biologically active form of nitrogen monoxide to a site of its intended activity, such as on a cell membrane in vivo. Nitrogen monoxide can exist in three forms: NO— (nitroxyl), NO. (nitric oxide) and NO⁺ (nitrosonium). NO. is a highly reactive short-lived species that is potentially toxic to cells. This is critical because the pharmacological efficacy of NO depends upon the form in which it is delivered. In contrast to the nitric oxide radical (NO.), nitrosonium (NO⁺) does not react with O₂ or O₂-species, and functionalities capable of transferring and/or releasing NO⁺ and NO— are also resistant to decomposition in the presence of many redox metals. Consequently, administration of charged NO equivalents (positive and/or negative) does not result in the generation of toxic by-products or the elimination of the active NO moiety.

The term “nitric oxide” encompasses uncharged nitric oxide (NO.) and charged nitrogen monoxide species, preferably charged nitrogen monoxide species, such as nitrosonium ion (NO⁺) and nitroxyl ion (NO—). The reactive form of nitric oxide can be provided by gaseous nitric oxide. The nitrogen monoxide releasing, delivering or transferring compounds have the structure F—NO, wherein F is a nitrogen monoxide releasing, delivering or transferring group, and include any and all such compounds which provide nitrogen monoxide to its intended site of action in a form active for its intended purpose.

The term “NO adducts” encompasses any nitrogen monoxide releasing, delivering or transferring compounds, including, for example, S-nitrosothiols, nitrites, nitrates, S-nitrothiols, sydnonimines, 2-hydroxy-2-nitrosohydrazines, (NONOates), (E)-alkyl-2-((E)-hydroxyimino)-5-nitro-3-hexeneamide (FK-409), (E)-alkyl-2-((E)-hydroxyimino)-5-nitro-3-hexeneamines, N-((2Z,3E)-4-ethyl-2-(hydroxyimino)-6-methyl-5-nitro-3-heptenyl)-3-pyridinecarboxamide (FR 146801), N-nitrosoamines, N-hydroxyl nitrosamines, nitrosimines, diazetine dioxides, oxatriazole 5-imines, oximes, hydroxylamines, N-hydroxyguanidines, hydroxyureas, benzofuroxanes, furoxans as well as substrates for the endogenous enzymes which synthesize nitric oxide.

Suitable NONOates include, but are not limited to, (Z)-1-(N-methyl-N-(6-(N-methyl-ammoniohexyl)amino))diazen-1-ium-1,2-diolate (“MAHMA/NO”), (Z)-1-(N-(3-ammoniopropyl)-N-(n-propyl)amino)diazen-1-ium-1,2-diolate (“PAPA/NO”), (Z)-1-(N-(3-aminopropyl)-N-(4-(3-aminopropylammonio)butyl)-amino)diazen-1-ium-1,2-diolate (spermine NONOate or “SPER/NO”) and sodium(Z)-1-(N,N-diethylamino)diazenium-1,2-diolate (diethylamine NONOate or “DEA/NO”) and derivatives thereof. NONOates are also described in U.S. Pat. Nos. 6,232,336, 5,910,316 and 5,650,447, the disclosures of which are incorporated herein by reference in their entirety. The “NO adducts” can be mono-nitrosylated, poly-nitrosylated, mono-nitrosated and/or poly-nitrosated at a variety of naturally susceptible or artificially provided binding sites for biologically active forms of nitrogen monoxide.

Suitable furoxanes include, but are not limited to, CAS 1609, C93-4759, C92-4678, S35b, CHF 2206, CHF 2363, R-substituted phenyl furoxans, di-R-substituted phenyl furoxans, and the like.

Suitable sydnonimines include, but are not limited to, molsidomine (N-ethoxycarbonyl-3-morpholinosydnonimine), SIN-1 (3-morpholinosydnonimine) CAS 936 (3-(cis-2,6-dimethylpiperidino)-N-(4-methoxybenzoyl)-sydnonimine, pirsidomine), C87-3754 (3-(cis-2,6-dimethylpiperidino)sydnonimine, linsidomine, C4144 (3-(3,3-dimethyl-1,4-thiazane-4-yl)sydnonimine hydrochloride), C89-4095 (3-(3,3-dimethyl-1,1-dioxo-1,4-thiazane-4-yl)sydnonimine hydrochloride, and the like.

Suitable oximes, include, but are not limited to, NOR-1, NOR-3, NOR-4, and the like.

Suitable nitroxide containing compounds, include, but are not limited to, substituted 2,2,6,6-tetramethyl-1-piperidinyloxy compounds, substituted 2,2,5,5-tetramethyl-3-pyrroline-1-oxyl compounds, substituted 2,2,5,5-tetramethyl-1-pyrrolidinyloxyl compounds, substituted 1,1,3,3-tetramethylisoindolin-2-yloxyl compounds, substituted 2,2,4,4-tetramethyl-1-oxazolidinyl-3-oxyl compounds, substituted 3-imidazolin-1-yloxy, 2,2,5,5-tetramethyl-3-imidazolin-1-yloxyl compounds, OT-551, 4-hydroxy-2,2,6,6-tetramethyl-1-piperidinyloxy (tempol), and the like. Suitable substituents, include, but are not limited to, aminomethyl, benzoyl, 2-bromoacetamido, 2-(2-(2-bromoacetamido)ethoxy)ethylcarbamoyl, carbamoyl, carboxy, cyano, 5-(dimethylamino)-1-naphthalenesulfonamido, ethoxyfluorophosphinyloxy, ethyl, 5-fluoro-2,4-dinitroanilino, hydroxy, 2-iodoacetamido, isothiocyanato, isothiocyanatomethyl, methyl, maleimido, maleimidoethyl, 2-(2-maleimidoethoxy)ethylcarbamoyl, maleimidomethyl, maleimido, oxo, phosphonooxy, and the like.

One group of NO adducts is the S-nitrosothiols, which are compounds that include at least one —S—NO group. These compounds include S-nitroso-polypeptides (the term “polypeptide” includes proteins and polyamino acids that do not possess an ascertained biological function, and derivatives thereof); S-nitrosylated amino acids (including natural and synthetic amino acids and their stereoisomers and racemic mixtures and derivatives thereof); S-nitrosylated sugars; S-nitrosylated, modified and unmodified, oligonucleotides (preferably of at least 5, and more preferably 5-200 nucleotides); straight or branched, saturated or unsaturated, aliphatic or aromatic, substituted or unsubstituted S-nitrosylated hydrocarbons; and S-nitroso heterocyclic compounds. S-nitrosothiols and methods for preparing them are described in U.S. Pat. Nos. 5,380,758 and 5,703,073; WO 97/27749; WO 98/19672; and Oae et al, Org. Prep. Proc. Int., 15(3):165-198 (1983), the disclosures of each of which are incorporated by reference herein in their entirety.

Another embodiment of the invention is S-nitroso amino acids where the nitroso group is linked to a sulfur group of a sulfur-containing amino acid or derivative thereof. Such compounds include, for example, S-nitroso-N-acetylcysteine, S-nitroso-captopril, S-nitroso-N-acetylpenicillamine, S-nitroso-homocysteine, S-nitroso-cysteine, S-nitroso-glutathione, S-nitroso-cysteinyl-glycine, and the like.

Suitable S-nitrosylated proteins include thiol-containing proteins (where the NO group is attached to one or more sulfur groups on an amino acid or amino acid derivative thereof) from various functional classes including enzymes, such as tissue-type plasminogen activator (TPA) and cathepsin B; transport proteins, such as lipoproteins; heme proteins, such as hemoglobin and serum albumin; and biologically protective proteins, such as immunoglobulins, antibodies and cytokines. Such nitrosylated proteins are described in WO 93/09806, the disclosure of which is incorporated by reference herein in its entirety. Examples include polynitrosylated albumin where one or more thiol or other nucleophilic centers in the protein are modified.

Other examples of suitable S-nitrosothiols include:

(i) HS(C(Rₑ)(R_{f}))ₘSNO;

(ii) ONS(C(Rₑ)(R_{f}))ₘRₑ; or

(iii) H₂N—CH(CO₂H)—(CH₂)ₘ—C(O)NH—CH(CH₂SNO)—C(O)NH—CH₂—CO₂H;

wherein m is an integer from 2 to 20;

Rₑ and R_{f} are each independently a hydrogen, an alkyl, a cycloalkoxy, a halogen, a hydroxy, an hydroxyalkyl, an alkoxyalkyl, an arylheterocyclic ring, an alkylaryl, an alkylcycloalkyl, an alkylheterocyclic ring, a cycloalkylalkyl, a cycloalkylthio, an arylalklythio, an arylalklythioalkyl, an alkylthioalkyl, a cycloalkenyl, an heterocyclicalkyl, an alkoxy, a haloalkoxy, an amino, an alkylamino, a dialkylamino, an arylamino, a diarylamino, an alkylarylamino, an alkoxyhaloalkyl, a sulfonic acid, a sulfonic ester, an alkylsulfonic acid, an arylsulfonic acid, an arylalkoxy, an alkylthio, an arylthio, a cyano, an aminoalkyl, an aminoaryl, an aryl, an arylalkyl, an alkylaryl, a carboxamido, an alkylcarboxamido, an arylcarboxamido, an amidyl, a carboxyl, a carbamoyl, an alkylcarboxylic acid, an arylcarboxylic acid, an alkylcarbonyl, an arylcarbonyl, an ester, a carboxylic ester, an alkylcarboxylic ester, an arylcarboxylic ester, a sulfonamido, an alkylsulfonamido, an arylsulfonamido, an alkylsulfonyl, an alkylsulfonyloxy, an arylsulfonyl, arylsulphonyloxy, a sulfonic ester, an alkyl ester, an aryl ester, a urea, a phosphoryl, a nitro, —U₃—V₅, V₆, —C(Rₒ)(Rₚ)ₖ₁—U₃—V₅, or Rₑ and R_{f} taken together with the carbons to which they are attached form a carbonyl, a methanthial, a heterocyclic ring, a cycloalkyl group, an aryl group, an oxime, a hydrazone, a bridged cycloalkyl group,

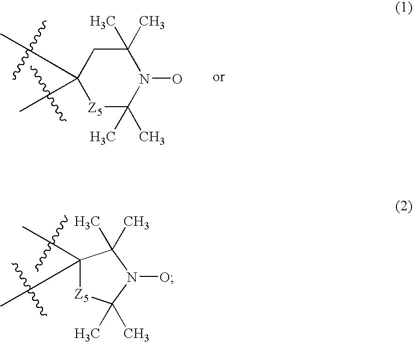

Rₒ and Rₚ are each independently a hydrogen, an alkyl, a cycloalkoxy, a halogen, a hydroxy, an hydroxyalkyl, an alkoxyalkyl, an alylheterocyclic ring, an alkylaryl, an alkylcycloalkyl, an alkylheterocyclic ring, a cycloalkylalkyl, a cycloalkylthio, an arylalklythio, an alylalklythioalkyl, an alkylthioalkyl a cycloalkenyl, an heterocyclicalkyl, an alkoxy, a haloalkoxy, an amino, an alkylamino, a dialkylamino, an arylamino, a diarylamino, an alkylarylamino, an alkoxyhaloalkyl, a sulfonic acid, a sulfonic ester, an alkylsulfonic acid, an arylsulfonic acid, an arylalkoxy, an alkylthio, an arylthio, a cyano an aminoalkyl, an aminoaryl, an aryl, an arylalkyl, an alkylaryl, a carboxamido, an alkylcarboxamido, an arylcarboxamido, an amidyl, a carboxyl, a carbamoyl, an alkylcarboxylic acid, an arylcarboxylic acid, an alkylcarbonyl, an arylcarbonyl, an ester, a carboxylic ester, an alkylcarboxylic ester, an arylcarboxylic ester, a sulfonamido, an alkylsulfonamido, an arylsulfonamido, an alkylsulfonyl, an alkylsulfonyloxy, an arylsulfonyl, arylsulphonyloxy, a sulfonic ester, an alkyl ester, an aryl ester, a urea, a phosphoryl, a nitro, —U₃—V₅, V₆, or Rₒ and Rₚ taken together with the carbons to which they are attached form a carbonyl, a methanthial, a heterocyclic ring, a cycloalkyl group, an aryl group, an oxime, an imine, a hydrazone, a bridged cycloalkyl group,

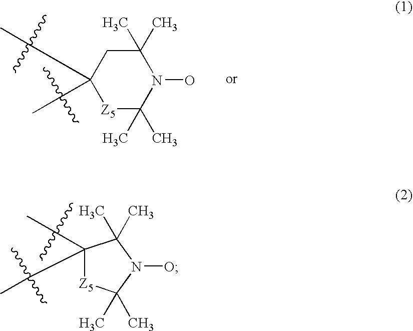

k₁ is an integer form 1 to 3;

U₃ is an oxygen, sulfur- or —N(Rₐ)Rᵢ;

V₅ is —NO or —NO₂ (i.e. an oxidized nitrogen);

Rₐ is a lone pair of electrons, a hydrogen or an alkyl group;

Rᵢ is a hydrogen, an alkyl, an aryl, an alkylcarboxylic acid, an arylcarboxylic acid, an alkylcarboxylic ester, an arylcarboxylic ester, an alkylcarboxamido, an arylcarboxamido, an alkylaryl, an alkylsulfinyl, an alkylsulfonyl, an alkylsulfonyloxy, an arylsulfinyl, an arylsulfonyl, arylsulphonyloxy, a sulfonamido, a carboxamido, a carboxylic ester, an aminoalkyl, an aminoaryl, —CH₂—C(U₃—V₅)(Rₑ)(R_{f}), a bond to an adjacent atom creating a double bond to that atom or —(N₂O₂—)⁻.M₁⁺, wherein M₁⁺ is an organic or inorganic cation.

In cases where Rₑ and R_{f} are independently a heterocyclic ring or taken together Rₑ and R_{f} are a heterocyclic ring, then Rᵢ can be a substituent on any disubstituted nitrogen contained within the radical wherein Rᵢ is as defined herein.

Nitrosothiols can be prepared by various methods of synthesis. In general, the thiol precursor is prepared first, then converted to the S-nitrosothiol derivative by nitrosation of the thiol group with NaNO₂ under acidic conditions (pH is about 2.5) which yields the S-nitroso derivative. Acids which can be used for this purpose include aqueous sulfuric, acetic and hydrochloric acids. The thiol precursor can also be nitrosylated by reaction with an organic nitrite such as tert-butyl nitrite, or a nitrosonium salt such as nitrosonium tetrafluoroborate in an inert solvent.

Another group of NO adducts for use in the invention, where the NO adduct is a compound that donates, transfers or releases nitric oxide, include compounds comprising at least one ON—O— or ON—N— group. The compounds that include at least one ON—O— or ON—N— group are preferably ON—O— or ON—N-polypeptides (the term “polypeptide” includes proteins and polyamino acids that do not possess an ascertained biological function, and derivatives thereof); ON—O— or ON—N-amino acids (including natural and synthetic amino acids and their stereoisomers and racemic mixtures); ON—O— or ON—N-sugars; ON—O— or —ON—N— modified or unmodified oligonucleotides (comprising at least 5 nucleotides, preferably 5-200 nucleotides); ON—O— or ON—N— straight or branched, saturated or unsaturated, aliphatic or aromatic, substituted or unsubstituted hydrocarbons; and ON—O—, ON—N— or ON—C-heterocyclic compounds. Preferred examples of compounds comprising at least one ON—O— or ON—N— group include butyl nitrite, isobutyl nitrite, tert-butyl nitrite, amyl nitrite, isoamyl nitrite, N-nitrosauines, N-nitrosamides, N-nitrosourea, N-nitrosoguanidines, N-nitrosocarbamates, N-acyl-N-nitroso compounds (such as, N-methyl-N-nitrosourea); N-hydroxy-N-nitrosamines, cupferron, alanosine, dopastin, 1,3-disubstitued nitrosiminobenzimidazoles, 1,3,4-thiadiazole-2-nitrosimines, benzothiazole-2(3H)-nitrosimines, thiazole-2-nitrosimines, oligonitroso sydnonimines, 3-alkyl-N-nitroso-sydnonimines and 2H-1,3,4-thiadiazine nitrosimines.

Another group of NO adducts for use in the invention include nitrates that donate, transfer or release nitric oxide, such as compounds comprising at least one O₂N—O—, O₂N—N— or O₂N—S— group. Preferred among these compounds are O₂N—O—, O₂N—N— or O₂N—S— polypeptides (the term “polypeptide” includes proteins and also polyamino acids that do not possess an ascertained biological function, and derivatives thereof); O₂N—O—, O₂N—N— or O₂N—S— amino acids (including natural and synthetic amino acids and their stereoisomers and racemic mixtures); O₂N—O—, O₂N—N— or O₂N—S— sugars; O₂N—O—, O₂N—N— or O₂N—S— modified and unmodified oligonucleotides (comprising at least 5 nucleotides, preferably 5-200 nucleotides); O₂N—O—, O₂N—N— or O₂N—S— straight or branched, saturated or unsaturated, aliphatic or aromatic, substituted or unsubstituted hydrocarbons; and O₂N—O—, O₂N—N— or O₂N—S— heterocyclic compounds. Preferred examples of compounds comprising at least one O₂N—O—, O₂N—N— or O₂N—S— group include isosorbide dinitrate, isosorbide mononitrate, clonitrate, erythrityl tetranitrate, mannitol hexanitrate, nitroglycerin, pentaerythritoltetranitrate, pentrinitrol, propatylnitrate and organic nitrates with a sulfhydryl-containing amino acid such as, for example SPM 3672, SPM 4757, SPM 5185, SPM 5186 and those disclosed in U.S. Pat. Nos. 5,284,872, 5,428,061, 5,661,129, 5,807,847 and 5,883,122 and in WO 97/46521, WO 00/54756 and in WO 03/013432, the disclosures of each of which are incorporated by reference herein in their entirety.

Another group of NO adducts are N-oxo-N-nitrosoamines that donate, transfer or release nitric oxide and are represented by the formula: R^{1″}R^{2″}N—N(O-M₁⁺)—NO, where R^{1″} and R^{2″} are each independently a polypeptide, an amino acid, a sugar, a modified or unmodified oligonucleotide, a straight or branched, saturated or unsaturated, aliphatic or aromatic, substituted or unsubstituted hydrocarbon, or a heterocyclic group, and where M₁⁺ is an organic or inorganic cation, such, as for example, an alkyl substituted ammonium cation or a Group I metal cation.

The invention is also directed to compounds that stimulate endogenous NO or elevate levels of endogenous endothelium-derived relaxing factor (EDRF) in vivo or are oxidized to produce nitric oxide and/or are substrates for nitric oxide synthase and/or cytochrome P450. Such compounds include, for example, L-arginine, L-homoarginine, and N-hydroxy-L-arginine, N-hydroxy-L-homoarginine, N-hydroxydebrisoquine, N-hydroxypentamidine including their nitrosated and/or nitrosylated analogs (e.g., nitrosated L-arginine, nitrosylated L-arginine, nitrosated N-hydroxy-L-arginine, nitrosylated N-hydroxy-L-arginine, nitrosated and nitrosylated L-homoarginine), N-hydroxyguanidine compounds, amidoxime, ketoximes, aldoxime compounds, that can be oxidized in vivo to produce nitric oxide. Compounds that may be substrates for a cytochrome P450, include, for example, imino(benzylamino)methylhydroxylamine, imino(((4-methylphenyl)methyl)amino)methylhydroxylamine, imino(((4-methoxyphenyl)methyl)amino) methylhydroxylamine, imino(((4-(trifluoromethyl)phenyl)methyl)amino) methylhydroxylamine, imino(((4-nitrophenyl)methyl)amino)methylhydroxylamine, (butylamino) iminomethylhydroxylamine, imino (propylamino)methylhydroxylamine, imino(pentylamino)methylhydroxylamine, imino (propylamino)methylhydroxylamine, imino ((methylethyl)amino)methylhydroxylamine, (cyclopropylamino) iminomethylhydroxylamine, imino-2-1,2,3,4-tetrahydroisoquinolyl methylhydroxylamine, imino(1-methyl(2-1,2,3,4-tetrahydroisoquinolyl))methylhydroxylamine, (1,3-dimethyl(2-1,2,3,4-tetrahydroisoquinolyl)) iminomethylhydroxylamine, (((4-chlorophenyl)methyl)amino)iminomethylhydroxylamine, ((4-chlorophenyl)amino)iminomethylhydroxylamine, (4-chlorophenyl)(hydroxyimino)methylamine, and 1-(4-chlorophenyl)-1-(hydroxyimino) ethane, and the like, precursors of L-arginine and/or physiologically acceptable salts thereof, including, for example, citrulline, ornithine, glutamine, lysine, polypeptides comprising at least one of these amino acids, inhibitors of the enzyme arginase (e.g., N-hydroxy-L-arginine and 2(S)-amino-6-boronohexanoic acid), nitric oxide mediators and/or physiologically acceptable salts thereof, including, for example, pyruvate, pyruvate precursors, α-keto acids having four or more carbon atoms, precursors of α-keto acids having four or more carbon atoms (as disclosed in WO 03/017996, the disclosure of which is incorporated herein in its entirety), and the substrates for nitric oxide synthase, cytokines, adenosin, bradykinin, calreticulin, bisacodyl, and phenolphthalein. EDRF is a vascular relaxing factor secreted by the endothelium, and has been identified as nitric oxide (NO) or a closely related derivative thereof (Palmer et al, Nature, 327:524-526 (1987); Ignarro et al, Proc. Natl. Acad. Sci. USA, 84:9265-9269 (1987)).

In another embodiment of the invention the combination of the β-adrenergic antagonists or ACE inhibitors of the invention (i.e. non-nitrosated and/or non-nitrosylated β-adrenergic antagonists and/or ACE inhibitors) with nitric oxide donor compounds do not include the combinations disclosed in US 2003/0216384, the disclosure of which is incorporated herein in its entirety.

The invention is also based on the discovery that compounds and compositions of the invention may be used in conjunction with other therapeutic agents for co-therapies, partially or completely, in place of other therapeutic agents, such as, for example, α-adrenergic receptor agonists, α-adrenergic receptor antagonists, angiotensin-converting enzyme (ACE) inhibitors, antimicrobial compounds, antioxidants, β-adrenergic antagonists, carbonic anhydrase inhibitors, hydralazine compounds, nonsteroidal antiinflammatory compounds (NSAIDs), prostaglandins, selective cyclooxygenase-2 (COX-2) inhibitors, steroids, and combinations of two or more thereof. The therapeutic agent may optionally be nitrosated and/or nitrosylated.

Suitable α-adrenergic receptor agonists, including, but are not limited to, agmatine, p-aminoclonidine, apraclonidine (IOPIDINE®), 2-(arylamino)imidazolidine derivatives, azepexole, azepin derivatives, such as for example, 2-amino-6-alkyl-4,5,7,8-tetrahydro-6H-thiazolo-(5,4,d) azepine, 2-amino-6-ethyl-4,5,7,8-tetrahydro-6H-thiazolo-(5,4,d) azepine, 2-amino-6-ethyl-4,5,7,8-tetrahydro-6H-oxazolo-(5,4,d) azepine, and the like; brimonidine, clonidine, clonidine derivatives, detomidine, dexmedetomidine, dipivefrin, dipivalylepinephrine, epinephrine, guanabenz, guanfacine, imidazolidine derivatives, such as, for example, 5-bromo-6-(2-imidazolidine-2-ylamino)quinoxaline, and the like; p-iodoclonidine, medetomidine, methoxamine (VASOXYL®), mephentermine, metaraminol (ARAMINE®), methyldopa, mitodrine, naphazoline (PRIVINE®, NAPHCON®), norepinephrine, oxymetazoline (AFRIN®, OCUCLEAR®), phenylepinephrine (NEOSYNEPHRINE®), rilmenidine, tetrahydrozoline (TYZINE®, VISINE®), tramazoline, xylazine, xylometazoline (OTRIVIN®), B-HT 920 (6-allyl-2-amino-5,6,7,8-tetrahydro-4H-thiazolo(4,5-d)-azepine, B-HT 933 and UK 14,304, and the like. Suitable α-adrenergic receptor agonists are described more fully in the literature, such as in Goodman and Gilman, The Pharmacological Basis of Therapeutics (9th Edition), McGraw-Hill, (1996); Merck Index on CD-ROM, 13^{th} Edition; STN Express, file phar and file registry, the disclosures of each of which are incorporated by reference herein in their entirety.

In some embodiment the α-adrenergic receptor agonist are aminoclonidine, apraclonidine (IOPIDINE®), brimonidine, clonidine and clonidine derivatives.

Suitable alpha-adrenergic receptor antagonists include but are not limited to, phentolamine, tolazoline, idazoxan, deriglidole, RX 821002, BRL 44408, BRL 44409, BAM 1303, labetelol, ifenprodil, rauwolscine, corynathine, raubascine, tetrahydroalstonine, apoyohimbine, akuammigine, β-yohimbine, yohimbol, yohimbine, pseudoyohimbine, epi-3α-yohimbine, 10-hydroxy-yohimbine, 11-hydroxy-yohimbine, tamsulosin, benoxathian, atipamezole, BE 2254, WB 4101, HU-723, tedisamil, mirtazipine, setiptiline, reboxitine, delequamine, naftopil, saterinone, SL 89.0591, ARC 239, urapidil, 5-methylurapidil, monatepi, haloperidol, indoramin, SB 216469, moxisylyte, trazodone, dapiprozole, efaroxan, Recordati 15/2739, SNAP 1069, SNAP 5089, SNAP 5272, RS 17053, SL 89.0591, KMD 3213, spiperone, AH 11110A, chloroethylclonidine, BMY 7378, niguldipine, and the like. Suitable alpha-adrenergic receptor antagonists are described more fully in the literature, such as in Goodman and Gilman, The Pharmacological Basis of Therapeutics (9th Edition), McGraw-Hill, 1995; and the Merck Index on CD-ROM, Thirteenth Edition; and on STN Express, file phar and file registry.

Suitable angiotensin-converting enzyme inhibitors (ACE inhibitors) include, but are not limited to, alacepril, benazepril (LOTENSIN®, CIBACEN®), benazeprilat, captopril, ceronapril, cilazapril, delapril, duinapril, enalapril, enalaprilat, fasidotril, fosinopril, fosinoprilat, gemopatrilat, glycopril, idrapril, imidapril, lisinopril, moexipril, moveltipril, naphthopidil, omapatrilat, pentopril, perindopril, perindoprilat, quinapril, quinaprilat, ramipril, ramiprilat, rentipril, saralasin acetate, spirapril, temocapril, trandolapril, trandolaprilat, urapidil, zofenopril, acylmercapto and mercaptoalkanoyl pralines, carboxyalkyl dipeptides, carboxyalkyl dipeptide, phosphinylalkanoyl pralines, registry no. 796406, AVE 7688, BP1.137, CHF 1514, E 4030, ER 3295, FPL-66564, MDL 100240, RL 6134, RL 6207, RL 6893, SA 760, S-5590, Z 13752A, and the like. Suitable angiotensin-converting enzyme inhibitors are described more fully in the literature, such as in Goodman and Gilman, The Pharmacological Basis of Therapeutics (9th Edition), McGraw-Hill, 1995; and the Merck Index on CD-ROM, Twelfth Edition, Version 12:1, 1996; and on STN Express, file phar and file registry.

In some embodiments the angiotensin-converting enzyme inhibitors are benazepril, captopril, enalapril, fosinopril, lisinopril, moexipril, quinapril, ramipril, trandolapril or trandolaprilat. In more particular embodiments the benazepril is administered as benazepril hydrochloride in an amount of about 5 milligrams to about 80 milligrams as a single dose or as multiple doses per day; the captopril is administered in an amount of about 12.5 milligrams to about 450 milligrams as a single dose or as multiple doses per day; the enalapril is administered as enalapril maleate in an amount of about 2.5 milligrams to about 40 milligrams as a single dose or as multiple doses per day; the fosinopril is administered as fosinopril sodium in an amount of about 5 milligrams to about 60 milligrams as a single dose or as multiple doses per day; the lisinopril is administered in an amount of about 2.5 milligrams to about 75 milligrams as a single dose or as multiple doses per day; the moexipril is administered as moexipril hydrochloride in an amount of about 7.5 milligrams to about 45 milligrams as a single dose or as multiple doses per day; the quinapril is administered as quinapril hydrochloride in an amount of about 5 milligrams to about 40 milligrams as single or multiple doses per day; the ramapril hydrochloride in an amount of about 1.25 milligrams to about 40 milligrams as single or multiple doses per day; the trandolapril is administered as in an amount of about 0.5 milligrams to about 4 milligrams as single or multiple doses per day; the trandolaprilat is administered as in an amount of about 0.5 milligrams to about 4 milligrams as single or multiple doses per day.

Suitable antimicrobial compounds, include, but are not limited to, acediasulfone, aceturate, acetyl sulfametossipirazine, acetyl sulfamethoxypyrazine, acranil, albendazole, alexidine, amatadine, ambazone, amdinocillin, amikacin, p-aminosalicylic acid, p-aminosalicylic acid hydrazine, amoxicillin, ampicillin, anisomycin, apalcillin, apicyclin, apramycin, arbekacin, argininsa, aspoxicillin, azidamfenicol, azidocillin, azithromycin, azlocillin, aztreonam, bacampicillin, bacitracin, benzoylpas, benzyl penicillin acid, benzyl sulfamide, bicozamycin, bipenam, brodimoprim, capreomycin, carbenicillin, carbomycin, cafazedone, carindacillin, carumonam, cefcapene pivoxil, cefaclor, cefadroxil, cefafroxil, cefamandole, cefatamet, cefatrizine, cefazedone, cefazolin, cefbuperazone, cefclidin, cefdinir, cefditoren, cefixime, cefinenoxime, cefmetazole, cefminox, cefodizime, cefonicid, cefoperazone, ceforanide, cefotaxime, cefotetan, cefotiam, cefoxitin, cefozopran, cefpimizole, cefpiramide, cefpirome, cefpodoxime proxetil, cefprozil, cefroxadine, cefsulodin, ceftazidime, cefteram, ceftezole, ceftibuten, ceftiofur, ceftizoxime, ceftriaxone, cefuroxime, cefuzonam, cephacetrile sodium, cephadrine, cephalexin, cephaloglycin, cephaloridine, cephalosporin C, cephalothin, cephapirin sodium, cephradine, chibrorifamycin, chloramphenicol, chlorotetracycline, cinoxacin, ciprofloxacin, claritromycin, clavulanic acid, clinafloxacin, clindamycin, clofazimine, clofoctal, clometocillin, clomocycline, cloxacillin, cloxyquin, colistin, cyclacilline, cycloserine, danoflaxcin, dapsone, deoxycycline, deoxydihydrostreptomycin, dibekacin, dicloxacillin, difloxacin, dihydrostreptomycin, dimetridazole, diminazene, dirirtomycin, duramycin, eflornithine, enrofloxacin, enviomycin, epicillin, erythromycin, etacillin, ethambutol, ethionamide, famcyclovir, fenbecillin, fleroxacin, flomoxef, floxacillin, flumequine, n-formamidoylthienamycin, furonazide, fortimycin, furazolium chloride, gentamycin, glyconiazide, gramicidin, grepafloxacin, guamecycline, halofuginone, hetacillin, homidium, hydroxyl-stilbamidine, ibostamycin, imidocarb, imipenam, ipronidazole, isoniazide, josamycin, inosine, kanamycin, lauroguadine, lenampicillin, lincomycin, lomefloxacin, loracarbef, lymecyclin, mafenide, mebendazole, meclocyclin, meropenem, metampicillin, metacicline, methacycline, methicillin sodium, metronidazole, 4′-(methylsulfamoyl) sulfanilanilide, mezlocillin, meziocillin, micronomycin, midecamycin A₁, minocycline, miocamycin, miokamycin, morfazinamide, moxalactam, mupirocin, myxin, nadifloxacin, nalidixic acid, negamycin, neomycin, netlimycin, nifurfoline, nifurpirinol, nifurprazine, nimorazole, nitroxoline, norfloxacin, novobiocin, ofloxacin, oleandomycin, opiniazide, oxacillin, oxophenarsine, oxolinic acid, oxytetracycline, panipenam, paromycin, pazufloxacin, pefloxacin, penicillin G potassium salt, penicillin N, penicillin O, penicillin V, penethamate hydroiodide, pentamidine, phenamidine, phenethicillin potassium salt, phenyl aminosalicyclate, pipacycline, pipemidic acid, piperacillin, pirlimycin, piromidic acid, pivampicillin, pivcefalexin, polymyxin B, profiromycin, propamidine, propicillin, protionamide, puraltadone, puromycin, pyrazinamide, pyrimethamine, quinacillin, quinacrine, quinapyramine, quintine, ribostamycin, rifabutine, rifamide, rifampin, rifamycin, rifanpin, rifapentine, rifaxymine, ritipenem, rokitamycin, rolitetracycline, rosamycin, rufloxacin, salazosulfadimidine, salinazid, sancycline, sarafloxacin, sedacamycin, secnidazole, sisomycin, sparfloxacin, spectinomycin, spiramycin, spiramycin I, spiramycin II, spiramycin III, stilbamidine, streptomycin, streptonicizid, sulbactam, sulbenicillin, succisulfone, sulfanilamide, sulfabenzamide, sulfacetamide, sulfachloropyridazine, sulfachrysoidine, sulfacytine, sulfadiazine, sulfadicramide, sulfadimethoxine, sulfadoxine, sulfadrazine, sulfaetidol, sulfafenazol, sulfaguanidine, sulfaguanole, sulfalene, sulfamerazine, sulfameter, sulfamethazine, sulfamethizole, sulfamethomidine, sulfamethoxazole, sulfamethoxypyridazine, sulfamethylthiazol, sulfamethylthiazole, sulfametrole, sulfamidochrysoidine, sulfamoxole, sulfanilamide, 4-sulfanilamido salicylic acid, 4-4′-sulfanilylbenzylamine, p-sulfanilylbenzylamine, 2-p-sulfinylanilinoethanol, sulfanilylurea, sulfoniazide, sulfaperine, sulfaphenazole, sulfaproxyline, sulfapyrazine, sulfapyridine, sulfathiazole, sulfaethidole, sulfathiourea, sulfisomidine, sulfasomizole, sulfasymazine, sulfisoxazole, 4,4′-sulfinyldianiline, N⁴-sulfanilylsulfanilamide, N-sulfanilyl-3,4-xylamide, sultamicillin, talampicillin, tambutol, taurolidine, teiclplanin, temocillin, tetracycline, tetroxoprim, thiabendazole, thiazolsulfone, tibezonium iodide, ticarcillin, tigemonam, timidazole, tobramycin, tosufloxacin, trimethoprim, troleandromycin, trospectomycin, trovafloxacin, tubercidine, miokamycin, oleandomycin, troleandromycin, vancomycin, verazide, viomycin, virginiamycin, zalcitabine, PA-1806 and PA-2794, and the like. Suitable antimicrobial compounds are described more fully in the literature, such as in Goodman and Gilman, The Pharmacological Basis of Therapeutics (9th Edition), McGraw-Hill, (1996); Merck Index on CD-ROM, 13^{th} Edition; STN Express, file phar and file registry, the disclosures of each of which are incorporated by reference herein in their entirety.

In some embodiments the antimicrobial compound amikacin, azithromycin, azetreonam, bacitracin, carbenicillin, cefazolin, cefoxitin, cephaloridine, chibrorifamycin, chloramphenicol, colistin, duramycin, n-formamidoylthienamycin, gentamycin, gramicidin, kanamycin, neomycin, penicillin G, polymyxin B, sisomicin, tetracyclines, tigecycline, tobramycin, vancomycin, PA-1806 and PA-2794.

In other embodiments the antimicrobial compound is an antiviral compound, including but not limited to, acyclovir, amatadine, cidofovir, cytarabine, didanosine, dideoxyadenosine, edoxudine, famciclovir, floxuridine, gancyclovir, idoxuridine, indanavir, kethoxal, lamivudine, MADU, penciclovir, podophyllotoxin, ribavirine, rimantadine, saquinavir, sorivudine, stavudine, trifluridine, valacyclovir, vidarabine, xenazoic acid, zalcitabine, zidovudine, and the like.

Suitable antioxidants include, but are not limited to, small-molecule antioxidants and antioxidant enzymes. Suitable small-molecule antioxidants include, but are not limited to, hydralazine compounds, glutathione, vitamin C, vitamin E, cysteine, N-acetyl-cysteine, β-carotene, ubiquinone, ubiquinol-10, tocopherols, coenzyme Q, superoxide dismutase mimetics, such as, for example, 2,2,6,6-tetramethyl-1-piperidinyloxy (TEMPO), DOXYL, PROXYL nitroxide compounds; 4-hydroxy-2,2,6,6-tetramethyl-1-piperidinyloxy (Tempol), M-40401, M-40403, M-40407, M-40419, M-40484, M-40587, M-40588, and the like. Suitable antioxidant enzymes include, but are not limited to, superoxide dismutase, catalase, glutathione peroxidase, NADPH oxidase inhibitors, such as, for example, apocynin, aminoguanidine, ONO 1714, S17834 (benzo(b)pyran-4-one derivative), and the like; xanthine oxidase inhibitors, such as, for example, allopurinol, oxypurinol, amflutizole, diethyldithiocarbamate, 2-styrylchromones, chrysin, luteolin, kaempferol, quercetin, myricetin, isorhamnetin, benzophenones such as 2,2′,4,4′-tetrahydroxybenzophenone, 3,4,5,2′,3′,4′-hexahydroxybenzophenone and 4,4′-dihydroxybenzophenone; benzothiazinone analogues such as 2-amino-4H-1,3-benzothiazine-4-one, 2-guanidino-4H-1,3-benzothiazin-4-one and rhodanine; N-hydroxyguanidine derivative such as, PR5 (1-(3,4-dimethoxy-2-chlorobenzylideneamino)-3-hydroxyguanidine); 6-formylpterin, and the like. The antioxidant enzymes can be delivered by gene therapy as a viral vertor and/or a non-viral vector. Suitable antioxidants are described more fully in the literature, such as in Goodman and Gilman, The Pharmacological Basis of Therapeutics (9th Edition), McGraw-Hill, 1995; and the Merck Index on CD-ROM, Thirteenth Edition; and on STN Express, file phar and file registry.

In some embodiments the antioxidants are apocynin, hydralazine compounds and superoxide dimutase mimetics.

Suitable antioxidants include, but are not limited to, small-molecule antioxidants and antioxidant enzymes. Suitable small-molecule antioxidants include, but are not limited to, hydralazine compounds, glutathione, vitamin C, vitamin E, cysteine, N-acetyl-cysteine, β-carotene, ubiquinone, ubiquinol-10, tocopherols, coenzyme Q, superoxide dismutase mimetics, such as, for example, 2,2,6,6-tetramethyl-1-piperidinyloxy (TEMPO), DOXYL, PROXYL nitroxide compounds; 4-hydroxy-2,2,6,6-tetramethyl-1-piperidinyloxy (Tempol), M-40401, M-40403, M-40407, M-40419, M-40484, M-40587, M-40588, and the like. Suitable antioxidant enzymes include, but are not limited to, superoxide dismutase, catalase, glutathione peroxidase, NADPH oxidase inhibitors, such as, for example, apocynin, aminoguanidine, ONO 1714, S17834 (benzo(b)pyran-4-one derivative), and the like; xanthine oxidase inhibitors, such as, for example, allopurinol, oxypurinol, amflutizole, diethyldithiocarbamate, 2-styrylchromones, chrysin, luteolin, kaempferol, quercetin, myricetin, isorhamnetin, benzophenones such as 2,2′,4,4′-tetrahydroxybenzophenone, 3,4,5,2′,3′,4′-hexahydroxybenzophenone and 4,4′-dihydroxybenzophenone; benzothiazinone analogues such as 2-amino-4H-1,3-benzothiazine-4-one, 2-guanidino-4H-1,3-benzothiazin-4-one and rhodanine; N-hydroxyguanidine derivative such as, PR5 (1-(3,4-dimethoxy-2-chlorobenzylideneamino)-3-hydroxyguanidine); 6-formylpterin, and the like. The antioxidant enzymes can be delivered by gene therapy as a viral vertor and/or a non-viral vector. Suitable antioxidants are described more fully in the literature, such as in Goodman and Gilman, The Pharmacological Basis of Therapeutics (9th Edition), McGraw-Hill, 1995; and the Merck Index on CD-ROM, Thirteenth Edition; and on STN Express, file phar and file registry.

In some embodiments the antioxidants are apocynin, hydralazine compounds and superoxide dimutase mimetics.

Suitable carbonic anhydrase inhibitors, include, but are not limited to, acetazolamide, brinzolamide, dorzolamide, ethoxzolamide, 6-hydroxy-2-benzothiazolesulfonamide, methazolamide, thiophene sulfonamide, an aromatic sulfonamide, an ester of 6-hydroxy-2-benzothiazolesulfonamide, an ester of 5-hydroxy-2-benzothiazolesulfonamide, and the like. Suitable carbonic anhydrase inhibitors are described more fully in the literature, such as in Goodman and Gilman, The Pharmacological Basis of Therapeutics (9th Edition), McGraw-Hill, 1995; and the Merck Index on CD-ROM, 13^{th} Edition; and on STN Express, file phar and file registry.

In some embodiments the carbonic anhydrase inhibitors are brinzolamide and dorzolamide.

Suitable hydralazine compounds include, but are not limited to, compounds having the formula:

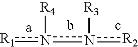

wherein a, b and c are independently a single or double bond; R₁ and R₂ are each independently a hydrogen, an alkyl, an ester or a heterocyclic ring, wherein alkyl, ester and heterocyclic rind are as defined herein; R₃ and R₄ are each independently a lone pair of electrons or a hydrogen, with the proviso that at least one of R₁, R₂, R₃ and R₄ is not a hydrogen. Exemplary hydralazine compounds include budralazine, cadralazine, dihydralazine, endralazine, hydralazine, pildralazine, todralazine, and the like. Suitable hydralazine compounds are described more fully in the literature, such as in Goodman and Gilman, The Pharmacological Basis of Therapeutics (9th Edition), McGraw-Hill, 1995; and the Merck Index on CD-ROM, Thirteenth Edition; and on STN Express, file phar and file registry.

In some embodiments the hydralazine compound is hydralazine or a pharmaceutically acceptable salt thereof such as hydralazine hydrochloride. In more particular embodiments the hydralazine is administered as hydralazine hydrochloride in an amount of about 10 milligrams to about 300 milligrams as a single dose or as multiple doses per day.

Suitable prostaglandins, include but are not limited to, naturally occurring prostaglandins such as, for example, arbaprostil, alprostadil, beraprost, carboprost, cloprostenol, dimoxaprost, enprostil, enisoprost, fluprostenol, fenprostalene, gemeprost, latanaprost, limaprost, meteneprost, mexiprostil, misoprostol, misoprost, misoprostol acid, nocloprost, ornoprostil, prostalene, PGE₁, PGE₂, PGF₁, PGF_{2α}, rioprostil, rosaprostol, remiprostol, sulprostone, trimoprostil, tiprostanide, travoprost, unoprostone, viprostol, viprostol. Suitable prostaglandins are described more fully in the literature, such as in Goodman and Gilman, The Pharmacological Basis of Therapeutics (9th Edition), McGraw-Hill, 1995; and the Merck Index on CD-ROM, 13^{th} Edition; and on STN Express, file phar and file registry.

In some embodiments the prostaglandins are cloprostenol, fluprostenol and travoprost.

Suitable NSAIDs include, but are not limited to, acetaminophen, acemetacin, aceclofenac, alminoprofen, amfenac, bendazac, benoxaprofen, bromfenac, bucloxic acid, butibufen, carprofen, cinmetacin, clopirac, diclofenac, etodolac, felbinac, fenclozic acid, fenbufen, fenoprofen, fentiazac, flunoxaprofen, flurbiprofen, ibufenac, ibuprofen, indomethacin, isofezolac, isoxepac, indoprofen, ketoprofen, lonazolac, loxoprofen, metiazinic acid, mofezolac, miroprofen, naproxen, oxaprozin, pirozolac, pirprofen, pranoprofen, protizinic acid, salicylamide, sulindac, suprofen, suxibuzone, tiaprofenic acid, tolmetin, xenbucin, ximoprofen, zaltoprofen, zomepirac, aspirin, acemetcin, bumadizon, carprofenac, clidanac, diflunisal, enfenamic acid, fendosal, flufenamic acid, flunixin, gentisic acid, ketorolac, meclofenamic acid, mefenamic acid, mesalamine, prodrugs thereof, and the like. Suitable NSAIDs are described more fully in the literature, such as in Goodman and Gilman, The Pharmacological Basis of Therapeutics (9th Edition), McGraw-Hill, 1995, Pgs. 617-657; the Merck Index on CD-ROM, 13^{th} Edition; and in U.S. Pat. Nos. 6,057,347 and 6,297,260 assigned to NitroMed. Inc., the disclosures of which are incorporated herein by reference in their entirety.

In some embodiments the NSAIDs are acetaminophen, diclofenac, flurbiprofen, ibuprofen, indomethacin, ketoprofen, naproxen or aspirin. In more particular embodiments the acetaminophen is administered in an amount of about 325 milligrams to about 4 grams as a single dose or as multiple doses per day; the diclofenac is administered in an amount of about 50 milligrams to about 250 milligrams as a single dose or as multiple doses per day; the flurbiprofen is administered in an amount of about 100 milligrams to about 300 milligrams as a single dose or as multiple doses per day; the ibuprofen is administered in an amount of about 400 milligrams to about 3.2 grams as a single dose or as multiple doses per day; the indomethacin is administered in an amount of about 25 milligrams to about 200 milligrams as a single dose or as multiple doses per day; the ketoprofen is administered in an amount of about 50 milligrams to about 300 milligrams as a single dose or as multiple doses per day; the naproxen is administered in an amount of about 250 milligrams to about 1.5 grams as a single dose or as multiple doses per day; the aspirin is administered in an amount of about 10 milligrams to about 2 grams as a single dose or as multiple doses per day.

Suitable steroids include, but are not limited to, 21-acetoxypregnenolone, alcolometasone, algestone, amcinonide, beclomethasone, betamethasone, budesonide, chlorprednisone, clobetasol, clobentasone, clocortolone, cloprednol, corticosterone, cortisine, corticazol (cortivatol), deflazacort, desonide, desoximetasone, dexamethasone, diflorasone, diflucortolone, difluprednate, enoxolone, fluzacort, flucloronide, flumethasone, flunisolide, flucinolone acetonide, fluocininide, fluocortin butyl, fluocortolone, fluorometholone, fluperolone acetate, fluprednidene acetate, fluprednisolone, flurandrenolide, fluticasone propionate, fluticasone propionate, formocortal, halcinonide, halobetasol propionate, halometasone, haloprednone acetate, hydrocortamate, hydrocortisone and its derivatives (such as phosphate, 21-sodium succinate and the like), hydrocortisone terbutate, isoflupredone, loteprednol etabonate, mazipredone, medrysone, meprednisone, methylprednisolone, mometasone furoate, paremethasone, prednicarbate, prednisolone and its derivatives (such as 21-stearoylglycolate, sodium phosphate and the like), prednisone, prednival, prednylidene and its derivatives (such as 21-diethylaminoactetate and the like), rimexolone, tixocortol, trimcinolone and its derivatives (such as acetonide, benetonide and the like), and the like. Suitable NSAIDs are described more fully in the literature, such as in Goodman and Gilman, The Pharmacological Basis of Therapeutics (9th Edition), McGraw-Hill, 1995, Pgs. 617-657; the Merck Index on CD-ROM, 13^{th} Edition; and in U.S. Pat. Nos. 6,057,347 and 6,297,260 assigned to NitroMed. Inc., the disclosures of which are incorporated herein by reference in their entirety.

In some embodiments the steroids are dexamethasone, fluorometholone, hydrocortisone, and prednisolone.

Suitable COX-2 inhibitors include, but are not limited to, nimesulide, celecoxib (CELEBREX®), etoricoxib (ARCOXIA®), flosulide, lumiracoxib (PREXIG®, COX-189), parecoxib (DYNSTAT®), rofecoxib (VIOXX®), tiracoxib (JTE-522), valdecoxib (BEXTRA®), ABT 963, BMS 347070, CS 502, DuP 697, GW-406381, NS-386, SC-57666, SC-58125, SC-58635, and the like, and mixtures of two or more thereof. Suitable COX-2 inhibitors are in U.S. Pat. Nos. 5,344,991, 5,380,738, 5,393,790, 5,409,944, 5,434,178, 5,436,265, 5,466,823, 5,474,995, 5,510,368, 5,536,752, 5,550,142, 5,552,422, 5,604,253, 5,604,260, 5,639,780, 5,932,598 and 6,633,272, and in WO 94/03387, WO 94/15723, WO 94/20480, WO 94/26731, WO 94/27980, WO 95/00501, WO 95/15316, WO 96/03387, WO 96/03388, WO 96/06840, WO 96/21667, WO 96/31509, WO 96/36623, WO 97/14691, WO 97/16435, WO 01/45703 and WO 01/87343, the disclosures of each of which are incorporated herein by reference in their entirety; and in the literature, such as in Goodman and Gilman, The Pharmacological Basis of Therapeutics (9th Edition), McGraw-Hill, 1995; and the Merck Index on CD-ROM, Thirteenth Edition; and on STN Express, file phar and file registry.

In some embodiments the COX-2 inhibitors are celecoxib, etoracoxib, lumiracoxib, paracoxib, rofecoxib or valdecoxib. In more particular embodiments the celecoxib is administered in an amount of about 100 milligrams to about 800 milligrams as a single dose or as multiple doses per day; the etoricoxib is administered in an amount of about 50 milligrams to about 200 milligrams as a single dose or as multiple doses per day; the lumiracoxib is administered in an amount of about 40 milligrams to about 1200 milligrams as a single dose or as multiple doses per day; the paracoxib is administered in an amount of about 20 milligrams to about 100 milligrams as a single dose or as multiple doses per day; the rofecoxib is administered in an amount of about 12.5 milligrams to about 50 milligrams as a single dose or as multiple doses per day; the valdecoxib is administered in an amount of about 10 milligrams to about 40 milligrams as a single dose or as multiple doses per day.

The invention provides methods for treating ophthalmic disorders by administering to the patient in need thereof a therapeutically effective amount of the compounds and/or compositions described herein. For example, the patient can be administered a therapeutically effective amount of at least one nitrosated and/or nitrosylated compound of the invention. In another embodiment, the patient can be administered a therapeutically effective amount of at least compound of the invention, that is optionally nitrosated and/or nitrosylated, and at least one nitric oxide donor compound. In yet another embodiment, the patient can be administered a therapeutically effective amount of at least one compound of the invention, that is optionally nitrosated and/or nitrosylated, and, at least one therapeutic agent, including but not limited to, such as, for example, α-adrenergic receptor agonists, α-adrenergic receptor antagonists, angiotensin-converting enzyme (ACE) inhibitors, antimicrobial compounds, antioxidants, β-adrenergic antagonists, carbonic anhydrase inhibitors, hydralazine compounds, nonsteroidal antiinflammatory compounds (NSAIDs), prostaglandins, selective cyclooxygenase-2 (COX-2) inhibitors, steroids, and combinations of two or more thereof. In another embodiment, the patient can be administered a therapeutically effective amount of at least one compound of the invention, that is optionally nitrosated and/or nitrosylated, and, at least one therapeutic agent, and, at least one nitric oxide donor compound. The compounds, which are optionally nitrosated and/or nitrosylated, nitric oxide donors, and/or therapeutic agents can be administered separately or as components of the same composition in one or more pharmaceutically acceptable carriers.

The invention provides methods for treating ophthalmic infection, glaucoma, ocular pain following corneal surgery, dry eye disorder, ocular hypertension, ocular bleeding, retinal diseases or disorders and lowering of intraocular pressure by administering to the patient in need thereof a therapeutically effective amount of the compounds and/or compositions described herein. For example, the patient can be administered a therapeutically effective amount of at least one nitrosated and/or nitrosylated compound of the invention. In another embodiment, the patient can be administered a therapeutically effective amount of at least compound of the invention, that is optionally nitrosated and/or nitrosylated, and at least one nitric oxide donor compound. In yet another embodiment, the patient can be administered a therapeutically effective amount of at least one compound of the invention, that is optionally nitrosated and/or nitrosylated, and, at least one therapeutic agent, including but not limited to, such as, for example, α-adrenergic receptor agonists, α-adrenergic receptor antagonists, angiotensin-converting enzyme (ACE) inhibitors, antimicrobial compounds, antioxidants, β-adrenergic antagonists, carbonic anhydrase inhibitors, hydralazine compounds, nonsteroidal antiinflammatory compounds (NSAIDs), prostaglandins, selective cyclooxygenase-2 (COX-2) inhibitors, steroids, and combinations of two or more thereof. In another embodiment, the patient can be administered a therapeutically effective amount of at least one compound of the invention, that is optionally nitrosated and/or nitrosylated, and, at least one therapeutic agent, and, at least one nitric oxide donor compound. In one embodiment the opthalmic disorder is ophthalmic infection, glaucoma, elevated intraocular pressure, ocular pain following corneal surgery, dry eye disorder, ocular hypertension, ocular bleeding, retinal diseases or disorders. The compounds that are optionally nitrosated and/or nitrosylated, nitric oxide donors, and/or therapeutic agents can be administered separately or as components of the same composition in one or more pharmaceutically acceptable carriers.

When administered separately, the compound of the invention, that is optionally nitrosated and/or nitrosylated, nitric oxide donor and/or therapeutic agent can be administered about the same time as part of the overall treatment regimen, i.e., as a combination therapy. “About the same time” includes administering the compound of the invention, which is optionally nitrosated and/or nitrosylated, simultaneously, sequentially, at the same time, at different times on the same day, or on different days, as long as they are administered as part of an overall treatment regimen, i.e., combination therapy or a therapeutic cocktail.

When administered in vivo, the compounds and compositions of the invention can be administered in combination with pharmaceutically acceptable carriers and in dosages described herein. When the compounds and compositions of the invention are administered as a combination of at least one compound of the invention and/or at least one nitrosated and/or nitrosylated compound of the invention and/or at least one nitric oxide donor and/or therapeutic agent, they can also be used in combination with one or more additional compounds which are known to be effective against the specific disease state targeted for treatment. The nitric oxide donors, therapeutic agents and/or other additional compounds can be administered simultaneously with, subsequently to, or prior to administration of the nitrosated and/or nitrosylated compound of the invention.

The compounds of the invention, can be incorporated into various types of pharmaceutical compositions, such as, for example, ophthalmic formulations for delivery to the eye (e.g., topically, intracamerally, or via an implant). The compounds are preferably incorporated into topical ophthalmic formulations, such as for example, solutions, suspensions, gels, ointments, implants, and the like. The compounds of the invention may be combined with opthalmologically acceptable preservatives, viscosity enhancers, penetration enhancers, buffers, sodium chloride, water to form an aqueous, sterile ophthalmic suspensions or solutions, and the like.

Suitable preservatives include, but are not limited to, benzalkonium chloride, thimerosal, chlorobutanol, methyl paraben, propyl paraben, phenylethyl alcohol, edetate disodium, sorbic acid, ONAMER®, and the like. The preservatives are typically employed at a concentration between about 0.001% and about 1.0% by weight. Appropriate co-solvents include, but are not limited to, Polysorbate 20, 60 and 80; Pluronic F-68, F-84 and P-103; Tyloxapol®; Cremophor® EL; sodium dodecyl sulfate; glycerol; PEG 400; propylene glycol; cyclodextrins, and the like. The co-solvents are typically employed at a concentration between about 0.01% and about 2% by weight. Viscosity enhancers are required as a viscosity greater than that of simple aqueous solutions may be desirable to increase ocular absorption of the active compound, to decrease variability in dispensing the formulations, to decrease physical separation of components of a suspension or emulsion of formulation and/or otherwise to improve the ophthalmic formulation. Suitable viscosity enhancers, include, but are not limited to, polyvinyl alcohol, methyl cellulose, hydroxy propyl carboxymethyl cellulose, hydroxymethylcellulose, hydroxyethylcellulose, hydroxypropylmethylcellulose, methylcellulose, polyvinylpyrrolidone, and the like. Gelling agents can also be used, including, but not limited to, gellan and xanthan gum, and the like. Viscosity enhancers are typically employed at a concentration between about 0.01% and about 2% by weight.

Ophthalmic solution formulations may be prepared by dissolving a compound in a physiologically acceptable isotonic aqueous buffer. Alternatively, the ophthalmic solution may include an opthalmologically acceptable surfactant to assist in dissolving the compound. Additionally for sterile ophthalmic ointment formulations, the compounds of the invention may be combined with a preservative in an appropriate vehicle, such as, mineral oil, liquid lanolin, or white petrolatum. Sterile ophthalmic gel formulations may be prepared by suspending the active ingredient in a hydrophilic base prepared from the combination of, for example, carbopol-974, and the like.

Various delivery systems are known and can be used to administer the compounds or compositions of the invention, including, for example, encapsulation in liposomes, microbubbles, emulsions, microparticles, microcapsules and the like. The required dosage can be administered as a single unit or in a sustained release form.

The bioavailability of the compositions can be enhanced by micronization of the formulations using conventional techniques such as grinding, milling, spray drying and the like in the presence of suitable excipients or agents such as phospholipids or surfactants.

Sustained release dosage forms of the invention may comprise microparticles and/or nanoparticles having a therapeutic agent dispersed therein or may comprise the therapeutic agent in pure, preferably crystalline, solid form. For sustained release administration, microparticle dosage forms comprising pure, preferably crystalline, therapeutic agents are preferred. The therapeutic dosage forms of this aspect of the invention may be of any configuration suitable for sustained release.

Nanoparticle sustained release therapeutic dosage forms are preferably biodegradable and, optionally, bind to the vascular smooth muscle cells and enter those cells, primarily by endocytosis. The biodegradation of the nanoparticles occurs over time (e.g., 30 to 120 days; or 10 to 21 days) in prelysosomic vesicles and lysosomes. Preferred larger microparticle therapeutic dosage forms of the invention release the therapeutic agents for subsequent target cell uptake with only a few of the smaller microparticles entering the cell by phagocytosis. A practitioner in the art will appreciate that the precise mechanism by which a target cell assimilates and metabolizes a dosage form of the invention depends on the morphology, physiology and metabolic processes of those cells. The size of the particle sustained release therapeutic dosage forms is also important with respect to the mode of cellular assimilation. For example, the smaller nanoparticles can flow with the interstitial fluid between cells and penetrate the infused tissue. The larger microparticles tend to be more easily trapped interstitially in the infused primary tissue, and thus are useful to deliver anti-proliferative therapeutic agents.

Particular sustained release dosage forms of the invention comprise biodegradable microparticles or nanoparticles. More particularly, biodegradable microparticles or nanoparticles are formed of a polymer containing matrix that biodegrades by random, nonenzymatic, hydrolytic scissioning to release therapeutic agent, thereby forming pores within the particulate structure.

The compounds and compositions of the invention can be formulated as pharmaceutically acceptable salt forms. Pharmaceutically acceptable salts include, for example, alkali metal salts and addition salts of free acids or free bases. The nature of the salt is not critical, provided that it is pharmaceutically-acceptable. Suitable pharmaceutically-acceptable acid addition salts may be prepared from an inorganic acid or from an organic acid. Examples of such inorganic acids include, but are not limited to, hydrochloric, hydrobromic, hydroiodic, nitric, carbonic, sulfuric and phosphoric acid and the like. Appropriate organic acids include, but are not limited to, aliphatic, cycloaliphatic, aromatic, heterocyclic, carboxylic and sulfonic classes of organic acids, such as, for example, formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, stearic, algenic, β-hydroxybutyric, cyclohexylaminosulfonic, galactaric and galacturonic acid and the like. Suitable pharmaceutically-acceptable base addition salts include, but are not limited to, metallic salts made from aluminum, calcium, lithium, magnesium, potassium, sodium and zinc or organic salts made from primary, secondary and tertiary amines, cyclic amines, N,N′-dibenzylethylenediamine, chloroprocaine, choline, diethanolamine, ethylenediamine, meglumine (N-methylglucamine) and procaine and the like. All of these salts may be prepared by conventional means from the corresponding compound by reacting, for example, the appropriate acid or base with the compound. In one embodiment, the pharmaceutically acceptable salts of the compounds of the invention do not include the nitrate salt.

While individual needs may vary, determination of optimal ranges for effective amounts of the compounds and/or compositions is within the skill of the art. Generally, the dosage required to provide an effective amount of the compounds and compositions, which can be adjusted by one of ordinary skill in the art, will vary depending on the age, health, physical condition, sex, diet, weight, extent of the dysfunction of the recipient, frequency of treatment and the nature and scope of the dysfunction or disease, medical condition of the patient, the route of administration, pharmacological considerations such as the activity, efficacy, pharmacokinetic and toxicology profiles of the particular compound used, whether a drug delivery system is used, and whether the compound is administered as part of a drug combination.

The amount of a given nitrosated and/or nitrosylated compound of the invention of the invention that will be effective in the treatment of a particular disorder or condition will depend on the nature of the disorder or condition, and can be determined by standard clinical techniques, including reference to Goodman and Gilman, supra; The Physician's Desk Reference, Medical Economics Company, Inc., Oradell, N.J., 1995; and Drug Facts and Comparisons, Inc., St. Louis, Mo., 1993. The precise dose to be used in the formulation will also depend on the route of administration, and the seriousness of the disease or disorder, and should be decided by the physician and the patient's circumstances.

The invention also provides pharmaceutical kits comprising one or more containers filled with one or more of the ingredients of the pharmaceutical compounds and/or compositions of the invention, including, at least, one or more of the novel compound of the invention, that is optionally nitrosated and/or nitrosylated, and one or more of the NO donors described herein. Associated with such kits can be additional therapeutic agents or compositions (e.g., α-adrenergic receptor agonists, α-adrenergic receptor antagonists, angiotensin-converting enzyme (ACE) inhibitors, antimicrobial compounds, antioxidants, β-adrenergic antagonists, carbonic anhydrase inhibitors, hydralazine compounds, nonsteroidal antiinflammatory compounds (NSAIDs), prostaglandins, selective cyclooxygenase-2 (COX-2) inhibitors, steroids, and the like, and combinations of two or more thereof), devices for administering the compositions, and notices in the form prescribed by a governmental agency regulating the manufacture, use or sale of pharmaceuticals or biological products which reflects approval by the agency of manufacture, use or sale for humans.

### EXAMPLES

The following non-limiting examples further describe and enable one of ordinary skill in the art to make and use the present invention. In each of the examples, flash chromatography was performed on 40 micron silica gel (Baker).

### Example 1

### Ethyl (2S)-2-(((1S)-2-((2S)-2-(((1S,2S,5S,6R)-6-(nitrooxy)-4,8-dioxabicyclo(3.3.0)oct-2-yl)oxycarbonyl)pyrrolidinyl)-1-methyl-2-oxoethyl)amino)-4-phenylbutanoate

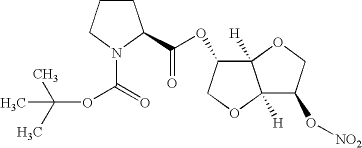

### 1a. (1S,2S,5S,6R)-6-(Nitrooxy)-4,8-dioxabicyclo(3.3.0)oct-2-yl (2S)-1-((tert-butyl)oxycarbonyl)pyrrolidine-2-carboxylate

N—BOC-L-Proline (Aldrich, 2.15 g, 9.99 mmole) was dissolved in dry methylene chloride (20 mL). Dicyclohexylcarbodiimide (DCC, 10.99 mmole, 1.1 eq) in methylene chloride was added at ambient temperature. Isosorbide 5-mononitrate (prepared as described in U.S. Pat. No. 4,431,830, 2.10 g, 10.99 mmole) and a catalytic amount of DMAP were added. After 2 hours, TLC (1:1 ethyl acetate/hexanes) indicated that the reaction was complete. The reaction mixture was filtered through a short pad of Celite and the clear filtrate was concentrated in vacuo to give a solid residue. The residue was triturated with a minimal amount of diethyl ether and then filtered. The crystals were washed with a minimal amount of cold diethyl ether to give the title compound (1.45 g, 37.4% yield) as a white solid. The filtrate was concentrated and triturated as described above, to give an additional 1.05 g (27.1%) of the title compound. Mp 109-111° C. ¹H NMR (300 MHz, CDCl₃) δ 5.33 (m, 1H), 5.26 (dd, J=14.5, 2.3 Hz, 1H), 4.96 (dt, J=12.9, 5.3 Hz, 1H), 4.47 (d, J=4.9 Hz, 1H), 4.26 (m, 1H), 4.04 (m, 3H), 3.90 (m, 2H), 2.24 (m, 1H), 1.91 (m, 3H), 1.46 (s, 4.5H), 1.42 (s, 4.5H). Mass spectrum (API-TIS) m/z 406 (MNH₄⁺), 389 (MH⁺).

### 1b. (1S,2S,5S,6R)-6-(Nitrooxy)-4,8-dioxabicyclo(3.3.0)oct-2-yl (2S)pyrrolidine-2-carboxylate

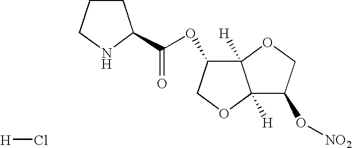

The product of Example 1a (1.00 g, 2.96 mmole) was added in one portion to hydrochloric acid in ethyl acetate (30 mL of a 14% w/w solution) cooled to 0° C. All of the solids went into solution after ca. 5 minutes. The reaction mixture was stirred at 0° C. for 30 minutes at which time TLC (1:1 ethyl acetate/hexanes) indicated that the reaction was complete. The solvent was removed in vacuo to give a clear oil. Trituration with methylene chloride and filtration of the solids gave the title compound (808 mg, 96.7% yield) as a white powdery solid. Mp 160° C. (dec). ¹H NMR (300 MHz, CDCl₃) δ 10.09 (bs, 1H), 9.07 (bs, 1H), 5.54 (td, J=5.4, 2.3 Hz, 1H), 5.23 (d, J=3.1 Hz, 1H), 5.01 (t, J=5.3 Hz, 1H), 4.51 (d, J=5.0 Hz, 1H), 4.39 (bt, J=7.7 Hz, 1H), 4.03 (m, 2H), 3.87 (m, 2H), 3.21 (m, 2H), 2.24 (m, 1H), 2.04 (m, 1H), 1.91 (m, 2H). Mass spectrum (API-TIS) m/z 289 (MH⁺).

### 1c. Ethyl (2S)-2-(((1S)-2-((2S)-2-(((1S,2S,5S,6R)-6-(nitrooxy)-4,8-dioxabicyclo(3.3.0)oct-2-yl)oxycarbonyl)pyrrolidinyl)-1-methyl-2-oxoethyl)amino)-4-phenylbutanoate

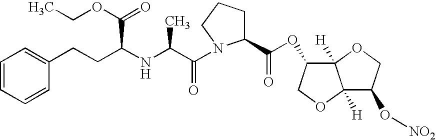

To the product of Example 1b (448 mg, 1.47 mmole) were added water (6 mL) and acetone (6 mL) were added and the solution was cooled to 0° C. under Argon. Solid sodium carbonate (233 mg, 2.20 mmole) was added followed by the addition of N-(1-(S)-ethoxycarbonyl-3-phenylpropyl)-L-alanine-N-carboxyanhydride (Lancaster Synthesis, 500 mg, 1.54 mmole) dissolved in 6mL of acetone at 0° C. The reaction mixture was stirred at 0° C. for 1 hour at which point TLC (1:1 ethyl acetate/hexanes) showed that the reaction was complete. The pH was adjusted to pH 5 using 5M HCl and the solvent was removed in vacuo to give a solid residue. The residue was dissolved in ethyl acetate and dried over sodium sulfate. The product was filtered, 1 g silica gel added and the solvent removed in vacuo. The product was subjected to flash chromatography eluting with 200 mL 1:1 ethyl acetate/hexanes and then 250 mL ethyl acetate. Concentration of the desired fractions gave the title compound (490 mg, 61.0% yield) as a colorless oil. ¹H NMR (300 MHz, CDCl₃) δ 7.27 (m, 2H), 7.18 (m, 3H), 5.33 (td, J=5.5, 2.7 Hz, 1H), 5.24 (d, J=2.7 Hz, 1H), 4.92 (t, J=5.3 Hz, 1H), 4.47 (m, 2H), 4.17 (qd, J=7.1, 1.2 Hz, 2H), 4.00 (m, 3H), 3.87 (m, 1H), 3.57 (bt, J=6.3 Hz, 2H), 3.52 (q, J=6.8 Hz, 1H), 3.22 (t, J=6.6 Hz, 1H), 2.67 (m, 2H), 2.21 (m, 2H), 1.98 (m, 5H), 1.28 (t, J=7.1 Hz, 3H), 1.26 (d, J=6.8 Hz, 3H). Mass spectrum (API-TIS) m/z 550 (MH⁺).

The disclosure of each patent, patent application and publication cited or described in the present specification is hereby incorporated by reference herein in its entirety.

Although the invention has been set forth in detail, one skilled in the art will appreciate that numerous changes and modifications can be made to the invention, and that such changes and modifications can be made without departing from the spirit and scope of the invention.

What is claimed is:

1. A method for treating an ophthalmic disorder in a patient in need thereof comprising administering to the patient a composition comprising a compound of Formula (I), (II), (III), (IV) or (V), or a pharmaceutically acceptable salt thereof,
wherein the compound of Formula (I) is: 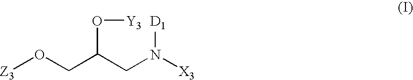
wherein:
X₃ is:
(1) —CH(CH₃)₂;
(2) —C(CH₃)₃; 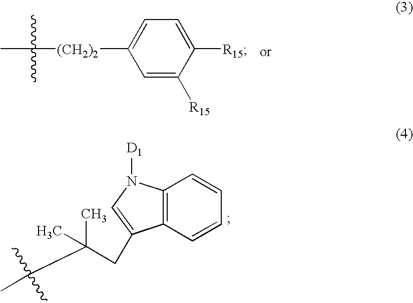
Y₃ is —C(O)—C₆H₅ or D₁;
Z₃ is: 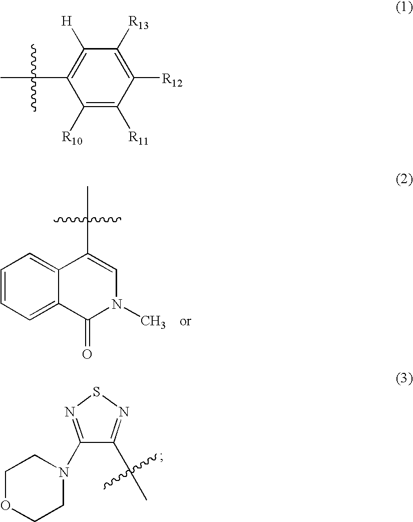
R₁₀ is:
(1) —C(O)—(CH₂)ₖ—CH₃;
(2) —O—CH₂—CH═CH₂;
(3) a hydrogen;
(4) methyl;
(5) methoxy;
(6) cyclopentyl;
(7) halo;
(8) —O—CH₂—C(O)—ND₁-CH₃;
(9) cyano;
(10) —CH₂—CH═CH₂; or 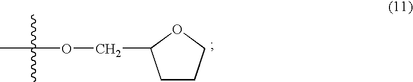
R₁₁ is a hydrogen, methyl or a halo; or
R₁₀ and R₁₁ taken together are W₄—U₄—V₄;
wherein W₄—U₄—V₄ is
(1) —CH═C(R₁₄)—ND₁-;
(2) —CH═CH—CH₂—;
(3) —CH₂—CH═CH—;
(4) —CH═CH—CH═CH—;
(5) —O—CH₂—CH(ONO₂)—CH₂—;
(6) —O—C(O)—CH═CH—;
(7) —(CH₂)₂—C(O)—ND₁-;
(8) —(CH₂)₃—C(O)—;
(9) —CH₂—CH(OD₁)-CH(OD₁)-CH₂—;
(10) —S—(CH₂)₃—; 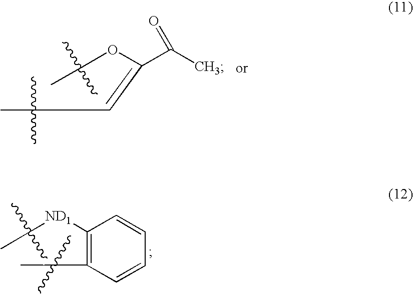
R₁₂ is:
(1) —ND₁-C(O)—(CH₂)ₖ—CH₃;
(2) —(CH₂)ₖ—C(O)—OD₁;
(3) —C(O)—(CH₂)ₖ—CH₃;
(4) halo;
(5) —ND₁-C(O)—N(C₂H₅)₂;
(6) —CH₂—C(O)—N(H)D₁;
(7) —O—C(O)—CH₃; 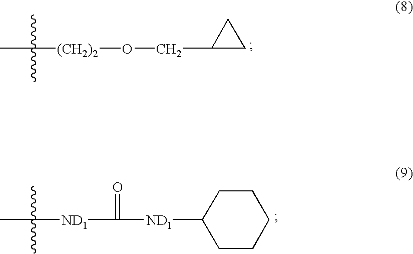
(10) —CH₂—O—(CH₂)₂—O—CH(CH₃)₂;
(11) methyl; or
(12) —(CH₂)₂—O—CH₃;
R₁₃ is a hydrogen, methyl or halo;
R₁₄ is a hydrogen or a lower alkyl;
R₁₅ at each occurrence is independently selected from —OCH₃, —OD₁, —NO₂, methyl or ND₁-S(O)₂—CH₃;
k is an integer from 0 to 4;
D₁ is a hydrogen, V₃ or K;
K is —(W₃)ₐ-E_{b}-(C(Rₑ)(R_{f}))ₚ₁-E_{c}-(C(Rₑ)(R_{f}))ₓ—(W₃)_{d}—(C(W)(R_{f}))_{y}—(W₃)ᵢ-Eⱼ-(W₃)_{g} (C(Rₑ)(R_{f}))_{z}—U₃—V₃;
V₃ is —NO or —NO₂;
a, b, c, d, g, i and j are each independently an integer from 0 to 3;
p₁, x, y and z are each independently an integer from 0 to 10;
W₃ at each occurrence is independently —C(O)—, —C(S)—, -T₃-, —(C(Rₑ)(R_{f}))ₕ—, an alkyl group, an aryl group, a heterocyclic ring, an arylheterocyclic ring, or —(CH₂CH₂O)_{q1}—;
E at each occurrence is independently -T₃-, an alkyl group, an aryl group, —(C(Rₑ)(R_{f}))ₕ—, a heterocyclic ring, an arylheterocyclic ring, or —(CH₂CH₂O)_{q1}—;
T₃ at each occurrence is independently a covalent bond, a carbonyl, an oxygen, —S(O)ₒ— or —N(Rₐ)Rᵢ;
h is an integer form 1 to 10;
q₁ is an integer from 1 to 5;
Rₑ and R_{f} are each independently a hydrogen, an alkyl, a cycloalkoxy, a halogen, a hydroxy, an hydroxyalkyl, an alkoxyalkyl, an arylheterocyclic ring, an alkylaryl, an alkylcycloalkyl, an alkylheterocyclic ring, a cycloalkylalkyl, a cycloalkylthio, an arylalklythio, an arylalklythioalkyl, an alkylthioalkyl a cycloalkenyl, an heterocyclicalkyl, an alkoxy, a haloalkoxy, an amino, an alkylamino, a dialkylamino, an arylamino, a diarylamino, an alkylarylamino, an alkoxyhaloalkyl, a sulfonic acid, a sulfonic ester, an alkylsulfonic acid, an arylsulfonic acid, an arylalkoxy, an alkylthio, an arylthio, a cyano, an aminoalkyl, an aminoaryl, an aryl, an arylalkyl, an alkylaryl, a carboxamido, a alkylcarboxamido, an arylcarboxamido, an amidyl, a carboxyl, a carbamoyl, an alkylcarboxylic acid, an arylcarboxylic acid, an alkylcarbonyl, an arylcarbonyl, an ester, a carboxylic ester, an alkylcarboxylic ester, an arylcarboxylic ester, a sulfonamido, an alkylsulfonamido, an arylsulfonamido, an alkylsulfonyl, an alkylsulfonyloxy, an arylsulfonyl, arylsulphonyloxy, a sulfonic ester, an alkyl ester, an aryl ester, a urea, a phosphoryl, a nitro, K or Rₑ and R_{f} taken together with the carbons to which they are attached form a carbonyl, a methanthial, a heterocyclic ring, a cycloalkyl group, an aryl group, an oxime, a hydrazone or a bridged cycloalkyl group;
U₃ at each occurrence is independently an oxygen, —S(O)ₒ— or —N(Rₐ)Rᵢ;
o is an integer from 0 to 2;
Rₐ is a lone pair of electrons, a hydrogen or an alkyl group;
Rᵢ is a hydrogen, an alkyl, an aryl, an alkylcarboxylic acid, an arylcarboxylic acid, an alkylcarboxylic ester, an arylcarboxylic ester, an alkylcarboxamido, an arylcarboxamido, an alkylaryl, an alkylsulfinyl, an alkylsulfonyl, an alkylsulfonyloxy, an arylsulfinyl, an arylsulfonyl, arylsulphonyloxy, a sulfonamido, a carboxamido, a carboxylic ester, an aminoalkyl, an aminoaryl, —CH₂—C(U₃—V₃)(Rₑ)(R_{f}), a bond to an adjacent atom creating a double bond to that atom, —(N₂O₂—)⁻.M₁⁺, wherein M₁⁺ is an organic or inorganic cation; and
with the proviso that the compounds of Formula (I) must contain at least one NO group, and/or at least one NO₂ group; wherein the at least one NO group and/or the at least one NO₂ group is linked to the compound through an oxygen atom, a nitrogen atom or a sulfur atom; and
the compound of Formula (II) is: 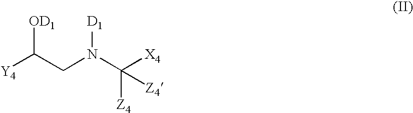
wherein:
Y₄ is: 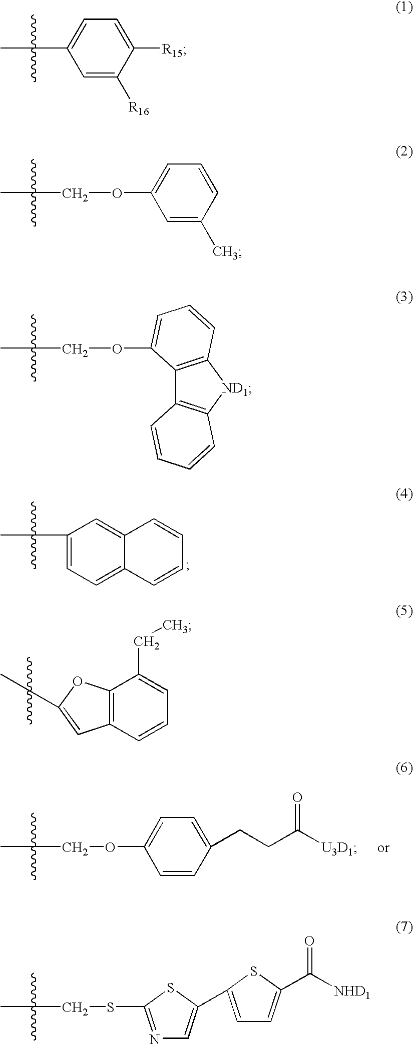
X₄ is:
(1) methyl; 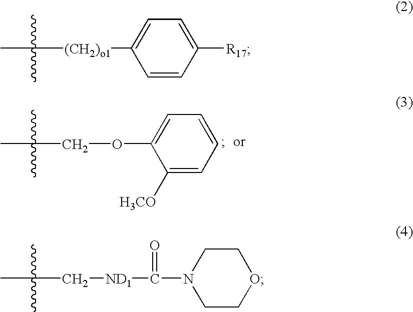
Z₄ and Z₄′ are independently selected from a methyl or a hydrogen;
R₁₆ is:
(1) hydrogen;
(2) —C(O)—N(D₁)H;
(3) —S(O)—CH₃; or
(4) —S(O)₂—N(D₁)H;
R₁₇ is a hydrogen, —OCH₃ or —NO₂;
o₁ is an integer from 0 to 2;
R₁₅ and D₁ are as defined herein; and
with the proviso that the compounds of Formula (II) must contain at least one NO group, and/or at least one NO₂ group; wherein the at least one NO group and/or the at least one NO₂ group is linked to the compound through an oxygen atom, a nitrogen atom or a sulfur atom; and
the compound of Formula (III) is: 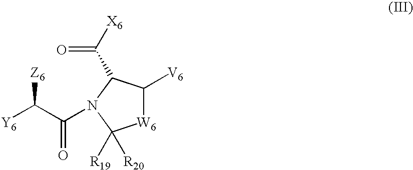
wherein:
X₆ is:
(1) —U₃D₁;
(2) —O—CH₂—CH₃; or 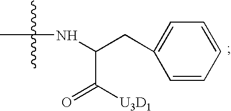
Y₆ is:
(1) —CH₂—S—R₂₁; 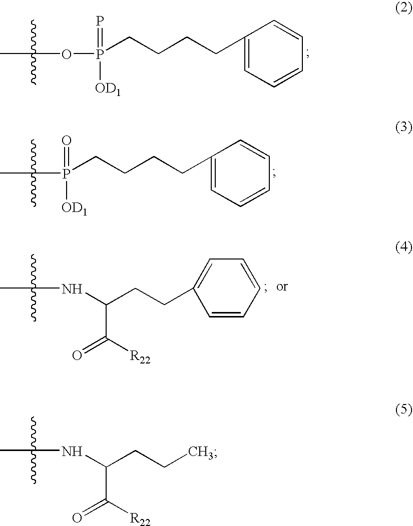
W₆ is: 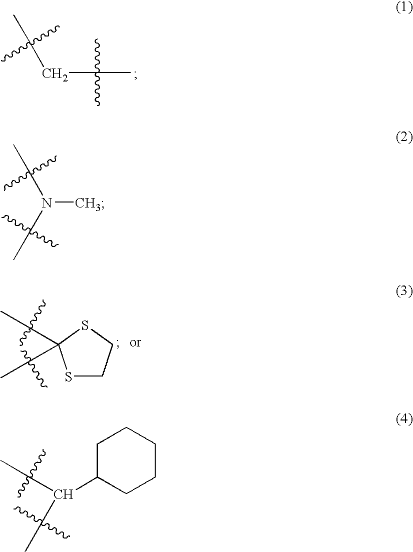
V₆ is a hydrogen;
Z₆ is:
(1) hydrogen;
(2) methyl; or
(3) —(CH₂)₄—N(H)D₁;
R₁₉ and R₂₀ are a hydrogen; or
R₁₉ and R₂₀ taken together are an oxo; or
R₂₀ and W₆ taken together are: 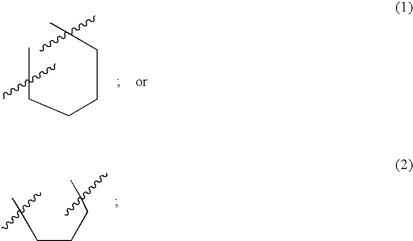
R₂₁ is:
(1) —C(O)—CH₂—CH₃;
(2) hydrogen;
(3) K; or 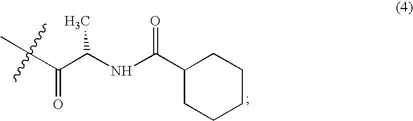
R₂₂ is —U₃D₁ or —OCH₂—CH₃;
D₁, U₃ and K are as defined herein; and
with the proviso that the compounds of Formula (III) must contain at least one NO group, and/or at least one NO₂ group; wherein the at least one NO group and/or the at least one NO₂ group is linked to the compound through an oxygen atom, a nitrogen atom or a sulfur atom; and
the compound of Formula (IV) is: 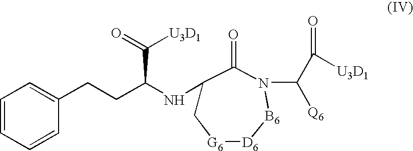
wherein:
B₆ is: 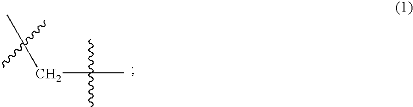
or
(2) a nitrogen;
G₆ is: 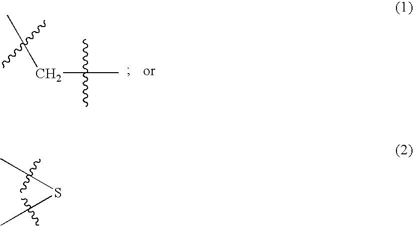
D₆ is: 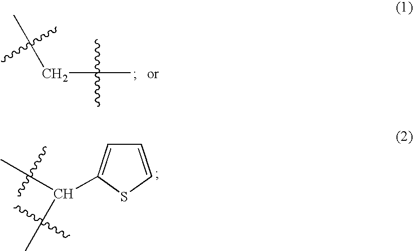
or B₆ and D₆ taken together form a phenyl ring;
Q₆ is a hydrogen; or
B₆ is a nitrogen and Q₆ is CH₂ and taken together form the ring: 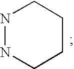
U₃ and D₁ are as defined herein; and
with the proviso that the compounds of Formula (IV) must contain at least one NO group, and/or at least one NO₂ group; wherein the at least one NO group and/or the at least one NO₂ group is linked to the compound through an oxygen atom, a nitrogen atom or a sulfur atom; and
the compound of Formula (V) is: 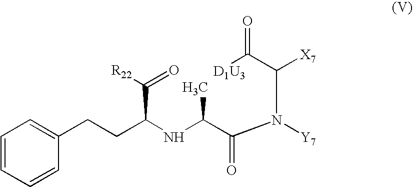
wherein:
X₇ is a hydrogen;
Y₇ is 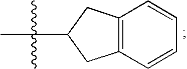
or X₇ and Y₇ taken together are: 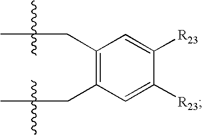
R₂₃ is a hydrogen or —OCH₃;
R₂₂, U₃ and D₁ are as defined herein; and
with the proviso that the compounds of Formula (V) must contain at least one NO group, and/or at least one NO₂ group; wherein the at least one NO group and/or the at least one NO₂ group is linked to the compound through an oxygen atom, a nitrogen atom or a sulfur atom.

2. The method of claim 1, wherein the composition further comprises a pharmaceutically acceptable carrier.

3. The method of claim 1, wherein the compound of Formula (I) is a nitrosated acebutolol, a nitrosylated acebutolol, a nitrosated and nitrosylated acebutolol, a nitrosated alprenolol, a nitrosylated alprenolol, a nitrosated and nitrosylated alprenolol, a nitrosated atenolol, a nitrosylated atenolol, a nitrosated and nitrosylated atenolol, a nitrosated befunolol, a nitrosylated befunolol, a nitrosated and nitrosylated befunolol, a nitrosated betaxolol, a nitrosylated betaxolol, a nitrosated and nitrosylated betaxolol, a nitrosated bevantolol, a nitrosylated bevantolol, a nitrosated and nitrosylated bevantolol, a nitrosated bisoprolol, a nitrosylated bisoprolol, a nitrosated and nitrosylated bisoprolol, a nitrosated bopindolol, a nitrosylated bopindolol, a nitrosated and nitrosylated bopindolol, a nitrosated bucindolol, a nitrosylated bucindolol, a nitrosated and nitrosylated bucindolol, a nitrosated bucumolol, a nitrosylated bucumolol, a nitrosated and nitrosylated bucumolol, a nitrosated bufetolol, a nitrosylated bufetolol, a nitrosated and nitrosylated bufetolol, a nitrosated bunitrolol, a nitrosylated bunitrolol, a nitrosated and nitrosylated bunitrolol, a nitrosated bupranolol, a nitrosylated bupranolol, a nitrosated and nitrosylated bupranolol, a nitrosated butofilolol, a nitrosylated butofilolol, a nitrosated and nitrosylated butofilolol, a nitrosated carazolol, a nitrosylated carazolol, a nitrosated and nitrosylated carazolol, a nitrosated carteolol, a nitrosylated carteolol, a nitrosated and nitrosylated carteolol, a nitrosated celiprolol, a nitrosylated celiprolol, a nitrosated and nitrosylated celiprolol, a nitrosated cetamolol, a nitrosylated cetamolol, a nitrosated and nitrosylated cetamolol, a nitrosated cloranolol, a nitrosylated cloranolol, a nitrosated and nitrosylated cloranolol, a nitrosated esmolol, a nitrosylated esmolol, a nitrosated and nitrosylated esmolol, a nitrosated indenolol, a nitrosylated indenolol, a nitrosated and nitrosylated indenolol, a nitrosated levobunolol, a nitrosylated levobunolol, a nitrosated and nitrosylated levobunolol, a nitrosated mepindolol, a nitrosylated mepindolol, a nitrosated and nitrosylated mepindolol, a nitrosated metipranolol, a nitrosylated metipranolol, a nitrosated and nitrosylated metipranolol, a nitrosated metoprolol, a nitrosylated metoprolol, a nitrosated and nitrosylated metoprolol, a nitrosated moprolol, a nitrosylated moprolol, a nitrosated and nitrosylated moprolol, a nitrosated nadolol, a nitrosylated nadolol, a nitrosated and nitrosylated nadolol, a nitrosated nipradilol, a nitrosylated nipradilol, a nitrosated and nitrosylated nipradilol, a nitrosated oxprenolol, a nitrosylated oxprenolol, a nitrosated and nitrosylated oxprenolol, a nitrosated penbutolol, a nitrosylated penbutolol, a nitrosated and nitrosylated penbutolol, a nitrosated pindolol, a nitrosylated pindolol, a nitrosated and nitrosylated pindolol, a nitrosated practolol, a nitrosylated practolol, a nitrosated and nitrosylated practolol, a nitrosated propranolol, a nitrosylated propranolol, a nitrosated and nitrosylated propranolol, a nitrosated talinolol, a nitrosylated talinolol, a nitrosated and nitrosylated talinolol, a nitrosated tertatolol, a nitrosylated tertatolol, a nitrosated and nitrosylated tertatolol, a nitrosated tilisolol, a nitrosylated tilisolol, a nitrosated and nitrosylated tilisolol, a nitrosated timolol, a nitrosylated timolol, a nitrosated and nitrosylated timolol, a nitrosated toliprolol, a nitrosylated toliprolol, a nitrosated and nitrosylated toliprolol, a nitrosated xibenolol, a nitrosylated xibenolol, a nitrosated and nitrosylated xibenolol; the compound of Formula (II) is a nitrosated amosulalol, a nitrosylated amosulalol, a nitrosated and nitrosylated amosulalol, a nitrosated arotinolol, a nitrosylated arotinolol, a nitrosated and nitrosylated arotinolol, a nitrosated bufuralol, a nitrosylated bufuralol, a nitrosated and nitrosylated bufuralol, a nitrosated carvedilol, a nitrosylated carvedilol, a nitrosated and nitrosylated carvedilol, a nitrosated dilevalol, a nitrosylated dilevalol, a nitrosated and nitrosylated dilevalol, a nitrosated labetalol, a nitrosylated labetalol, a nitrosated and nitrosylated labetalol, a nitrosated landiolol, a nitrosylated landiolol, a nitrosated and nitrosylated landiolol, a nitrosated nifenalol, a nitrosylated nifenalol, a nitrosated and nitrosylated nifenalol, a nitrosated pronethalol, a nitrosylated pronethalol, a nitrosated and nitrosylated pronethalol, a nitrosated sotalol, a nitrosylated sotalol, a nitrosated and nitrosylated sotalol, a nitrosated sulfinalol, a nitrosylated sulfinalol, a nitrosated and nitrosylated sulfinalol; the compound of Formula (III) is a nitrosated alacepril, a nitrosylated alacepril, a nitrosated and nitrosylated alacepril, a nitrosated captopril, a nitrosylated captopril, a nitrosated and nitrosylated captopril, a nitrosated ceronapril, a nitrosylated ceronapril, a nitrosated and nitrosylated ceronapril, a nitrosated enalapril, a nitrosylated enalapril, a nitrosated and nitrosylated enalapril, a nitrosated enalaprilat, a nitrosylated enalaprilat, a nitrosated and nitrosylated enalaprilat, a nitrosated fosinopril, a nitrosylated fosinopril, a nitrosated and nitrosylated fosinopril, a nitrosated imidapril, a nitrosylated imidapril, a nitrosated and nitrosylated imidapril, a nitrosated lisinopril, a nitrosylated lisinopril, a nitrosated and nitrosylated lisinopril, a nitrosated moveltipril, a nitrosylated moveltipril, a nitrosated and nitrosylated moveltipril, a nitrosated perindopril, a nitrosylated perindopril, a nitrosated and nitrosylated perindopril, a nitrosated ramipril, a nitrosylated ramipril, a nitrosated and nitrosylated ramipril, a nitrosated spirapril, a nitrosylated spirapril, a nitrosated and nitrosylated spirapril, a nitrosated trandolapril, a nitrosylated trandolapril, a nitrosated and nitrosylated trandolapril; the compound of Formula (IV) is a nitrosated benazepril, a nitrosylated benazepril, a nitrosated and nitrosylated benazepril, a nitrosated cilazapril, a nitrosylated cilazapril, a nitrosated and nitrosylated cilazapril, a nitrosated temocapril, a nitrosylated temocapril, a nitrosated and nitrosylated temocapril; the compound of Formula (V) is a nitrosated delapril, a nitrosylated delapril, a nitrosated and nitrosylated delapril, a nitrosated moexipril, a nitrosylated moexipril, a nitrosated and nitrosylated moexipril, a nitrosated quinapril, a nitrosylated quinapril, a nitrosated and nitrosylated quinapril, or a pharmaceutically acceptable salt thereof.

4. The method of claim 1, wherein K is:
(1) —Y—(CR₄R₄′)ₚ-T-(CR₄R₄′)ₚ—ONO₂; 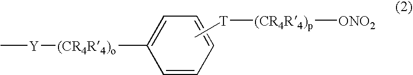
wherein T is ortho, meta or para; 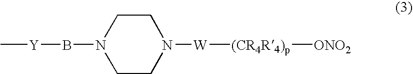
(4) —Y—(CR₄R₄′)ₚ—V—B-T-(CR₄R₄′)ₚ—ONO₂;
(5) —Y—(CR₄R₄′)ₚ-T-C(O)—(CR₄R₄′)ₒ—(CH₂)—ONO₂;
(6) —Y—(CR₄R₄′)ₚ—C(Z)-(CH₂)_{q}-T-(CR₄R₄′)_{q}—(CH₂)—ONO₂;
(7) —Y—(CR₄R₄′)ₚ-T-(CH₂)_{q}—V—(CR₄R₄′)_{q}—(CH₂)—ONO₂;
(8) —Y—(CR₄R₄′)ₚ—V—(CH₂)_{q}—V—(CR₄R₄′)_{q}—(CH₂)—ONO₂;
(9) —Y—(CR₄R₄′)ₒ—(W)_{q}—(CR₄R₄′)ₒ—(CH₂)—ONO₂;
(10) —NRⱼ—O—(CH₂)ₒ—V—(CR₄R₄′)_{q}—(CH₂)—ONO₂;
(11) —NRⱼ—O—(CH₂)ₒ—(W)_{q}—(CR₄R₄′)_{q}—(CH₂)—ONO₂;
(12) —O—NRⱼ—(CH₂)ₒ—(W)_{q}—(CR₄R₄′)_{q}—(CH₂)—ONO₂;
(13) —Y—(CH₂)ₒ—(W)_{q}—(CH₂)ₒ—V—(CR₄R₄′)ₒ-Q′-(CR₄R₄′)ₙ—(CH₂)—ONO₂;
(14) —Y—(CR₄R₄′)ₚ—V—(CH₂)ₒ—(W)_{q}—(CR₄R₄′)_{q}—(CH₂)—ONO₂;
(15) —O—NRⱼ—(CH₂)ₒ—V—(CR₄R₄′)_{q}—(CH₂)—ONO₂;
(16) —Y—(CR₄R₄′)ₒ-Q′-(CR₄′)ₒ—V—(CR₄R₄′)ₒ—(CH₂)—ONO₂;
(17) —Y—(CR₄R₄′)ₒ-Q′-(CR₄R₄′)ₒ—(W)_{q}—(CR₄R₄′)ₒ—(CH₂)—ONO₂;
(18) —Y—(CR₄R₄′)ₚ-T-(C₄R₄′)ₚ-Q′-(CR₄R₄′)ₒ—(CH₂)—ONO₂;
(19) —Y—(CR₄R₄′)_{q}—C(Z)-(CR₄R₄′)ₒ—(CH₂)—ONO₂;
(20) —Y—(CR₄R₄′)ₚ-Q′-(CR₄R₄′)ₒ—(CH₂)—ONO₂;
(21) —Y—(CR₄R₄′)_{q}—P(O)MM′;
(22) —Y—(CR₄R₄′)ₒ-Q′-(CR₄R₄′)ₒ—(CH₂)—ONO₂;
(23) —Y—(CR₄R₄′)ₒ-Q′-(CR₄R₄′)ₒ-T-(CR₄R₄′)ₒ—(CH₂)—ONO₂;
(24) —Y—(CR₄R₄′)_{q}—(W)_{q}—(CR₄R₄′)ₒ-Q′-(CR₄R₄′)ₒ—(CH₂)—ONO₂;
(25) —Y—(CR₄R₄′)_{q}—V—(CR₄R₄′)ₒ-Q′-(CR₄R₄′)ₒ—(CH₂)—ONO₂;
(26) —Y—(CR₄R₄′)ₚ-(T)ₒ-(W)_{q}—(CR₄R₄′)ₒ—(CH₂)—ONO₂;
(27) —Y—(CR₄R₄′)ₚ—(W)_{q}-(T)ₒ-(CR₄R₄′)ₒ—(CH₂)—ONO₂;
(28) —Y—(CR₄R₄′)_{q}—C(Z)-V—(CR₄R₄′)_{q}—(CH₂)—ONO₂;
(29) —Y—(CR₄R₄′)ₒ—C(R₄)(ONO₂)—(CR₄R₄′)_{q}-(T)ₒ-(W)_{q}-(T)ₒ-(CR₄R₄′)ₒ—R₅;
(30) —Y—(CR₄R₄′)ₒ—V—(CR₄R₄′)ₒ-Q′-(CR₄R₄′)ₒ—(CH₂)—ONO₂;
(31) —Y—(CR₄R₄′)_{q}—C(Z)-Q′-(CR₄R₄′)ₒ—(CH₂)—ONO₂;
(32) —Y—(CR₄R₄′)ₚ—V—(CR₄R₄′)ₚ—(CH₂)—ONO₂;
(33) —Y—(CR₄R₄′)ₚ—V—(CH₂)_{q}-(T)ₒ-(CR₄R₄′)_{q}—(CH₂)—ONO₂;
(34) —Y—(CR₄R₄′)ₚ-(T)ₒ-Q′-(T)ₒ-(CR₄R₄′)_{q}—(CH₂)—ONO₂;
(35) —Y—(CR₄R₄′)_{q}—C(Z)-(CR₄R₄′)_{q}—V—(CR₄R₄′)ₒ-Q′-(CR₄R₄′)ₒ—(CH₂)—ONO₂;
(36) —Y—(CR₄R₄′)_{q}—C(Z)-(CR₄R₄′)_{q}—(W)_{q}—(CR₄R₄′)ₒ-Q′-(CR₄R₄′)ₒ—(CH₂)—ONO₂;
(37) —NRⱼ—O—(CH₂)ₒ—V—(CR₄R₄′)ₒ-Q′-(CH₂)—ONO₂;
(38) —NRⱼ—O—(CH₂)ₒ—(W)_{q}—(CR₄R₄′)ₒ-Q′-(CH₂)—ONO₂;
(39) —O—-NRⱼ—(CH₂)ₒ—(W)_{q}—(CR₄′)ₒ-Q′-(CH₂)—ONO₂;
(40) —O—NRⱼ—(CH₂)ₒ—V—(CR₄R₄′)ₒ-Q′-(CH₂)—ONO₂;
(41) —NRⱼ—NRⱼ—(CR₄R₄′)ₚ—(W)_{q}-(T)ₒ-(CR₄R₄′)ₒ—(CH₂)—ONO₂;
(42) —Y—(CR₄R₄′)ₒ-Q′-(CR₄R₄′)ₒ—ONO₂; or
(43) —Y—(CR₄R₄′)ₒ—V—(CR₄R₄′)ₒ-Q-(CR₄R₄′)ₒ—ONO₂;
R₄ and R₄′ at each occurrence are independently a hydrogen, lower alkyl group, —OH, —CH₂OH, —ONO₂, —NO₂ or —CH₂ONO₂; or R₄ and R₄′ taken together with the carbon atom to which they are attached are a cycloalkyl group or a heterocyclic ring;
V is —C(O)-T-, -T-C(O)—, -T-C(O)-T or T-C(O)—C(O)-T;
W is a covalent bond or a carbonyl group;
T at each occurrence is independently an oxygen, (S(O)ₒ)ₒ or NRⱼ;
Rⱼ is a hydrogen, an alkyl group, an aryl group, a heterocyclic ring, an alkylcarbonyl group, an alkylaryl group, an alkylsulfinyl group, an alkylsulfonyl group, an arylsulfinyl group, an arylsulfonyl group, a sulfonamido group, a N-alkylsulfonamido group, a N,N-diarylsulfonamido group, a N-arylsulfonamido group, a N-alkyl-N-arylsulfonamido group, a carboxamido group or a hydroxyl group;
p at each occurrence is independently an integer from 1 to 6;
q at each occurrence is independently an integer from 1 to 3;
o at each occurrence is independently an integer from 0 to 2;
Y is independently a covalent bond, a carbonyl, an oxygen, —S(O)ₒ— or —NRⱼ;
B is either phenyl or (CH₂)ₒ;
Q′ is a cycloalkyl group, a heterocyclic ring or an aryl group;
Z is (═O), (═N—OR₅), (═N—NR₅R′₅) or (═CR₅R′₅);
M and M′ are each independently —O⁻H₃N⁺—(CR₄R′₄)_{q}—CH₂ONO₂ or -T-(CR₄R′₄)ₒ—CH₂ONO₂; and
R₅ and R₅′ at each occurrence are independently a hydrogen, a hydroxyl group, an alkyl group, an aryl group, an alkylsulfonyl group, an arylsulfonyl group, a carboxylic ester, an alkylcarbonyl group, an arylcarbonyl group, a carboxamido group, an alkoxyalkyl group, an alkoxyaryl group, a cycloalkyl group or a heterocyclic ring.

5. The method of claim 1, wherein K is: 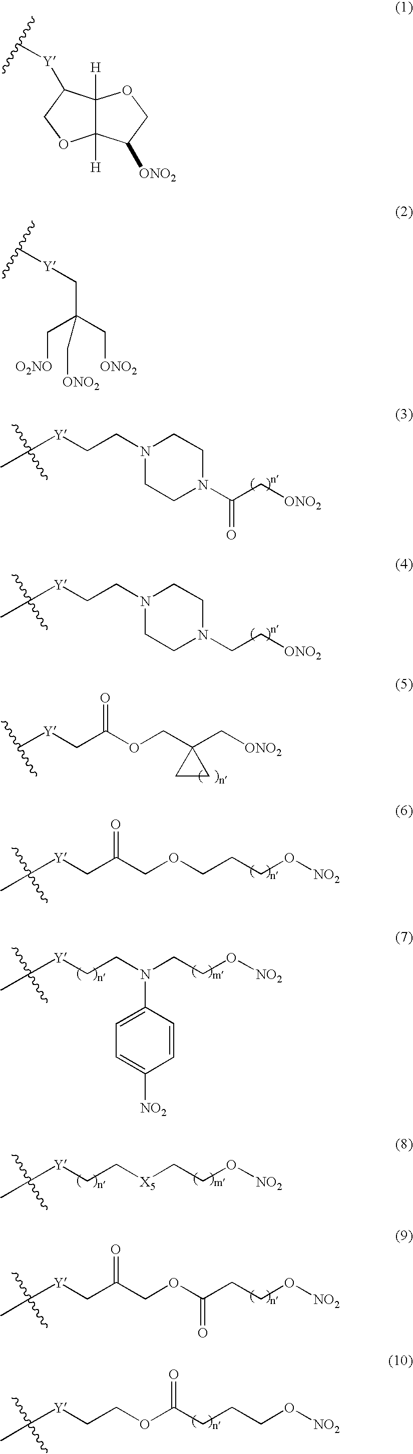 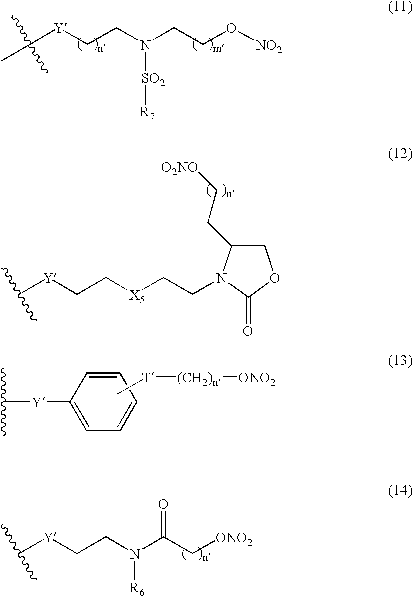
wherein T′ maybe oitho meta or para 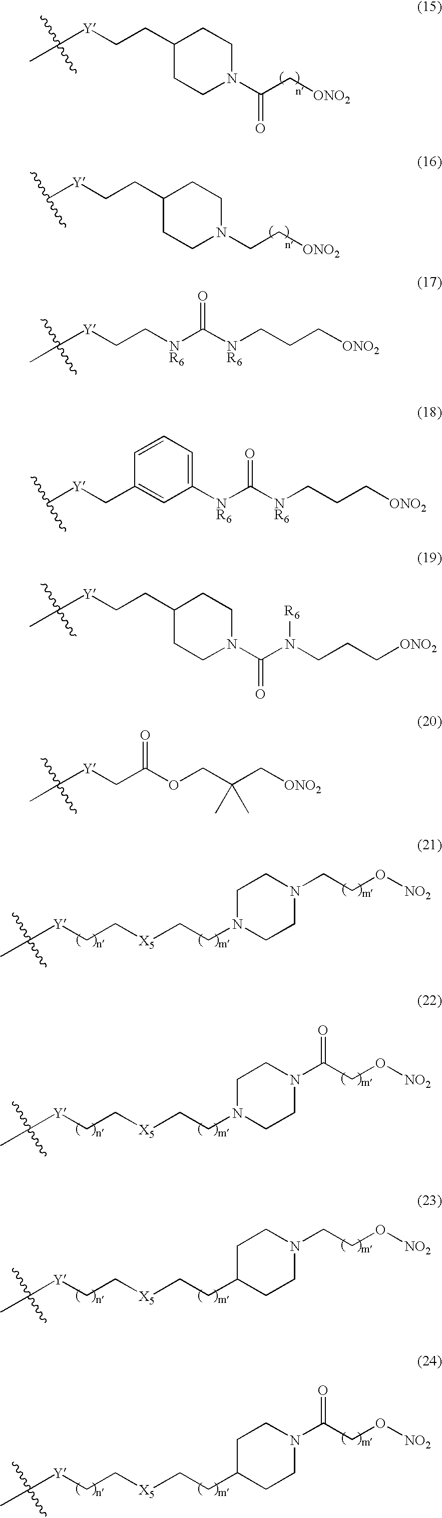 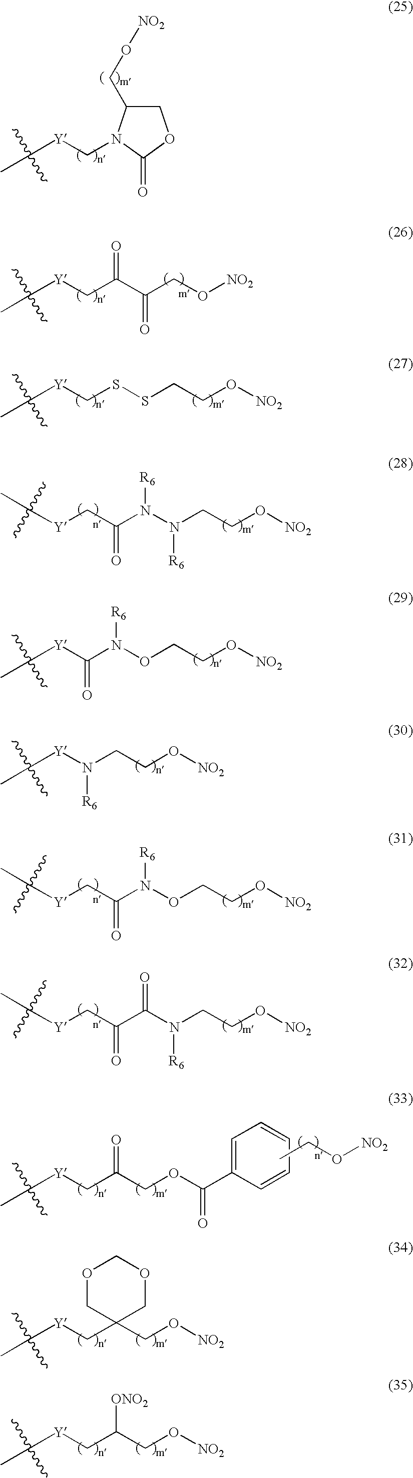 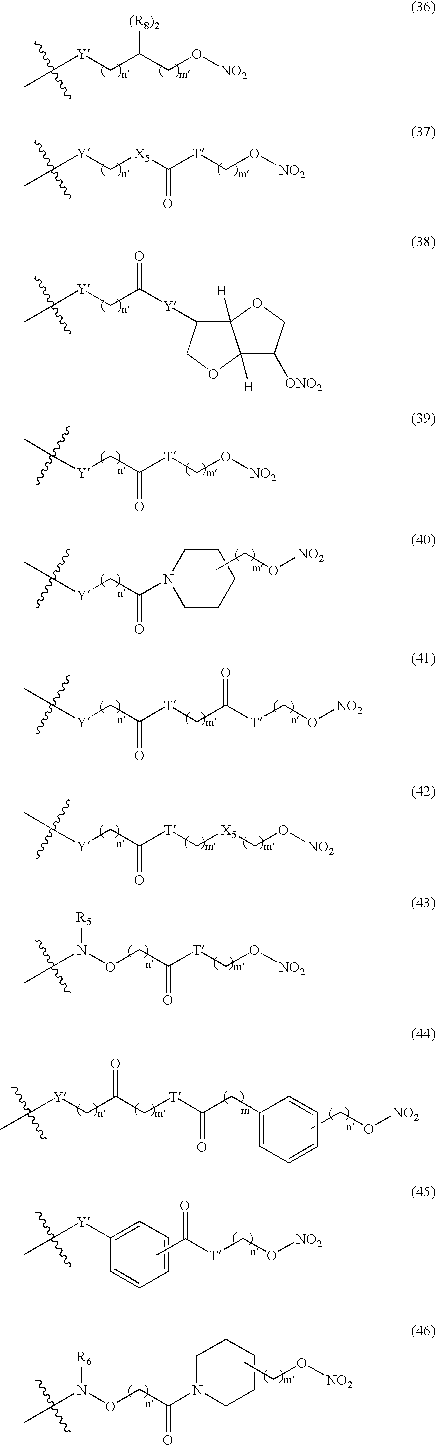 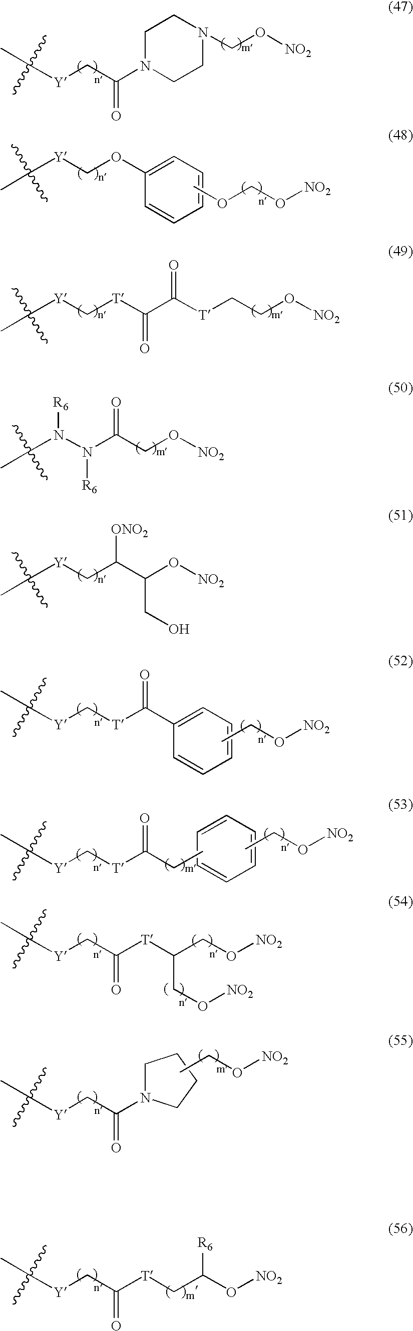
wherein:
Y′ a covalent bond, a carbonyl, an oxygen, —S(O)ₒ— or —NR₆;
T′ is oxygen, sulfur or NR₆;
X₅ is oxygen, (S(O)ₒ)ₒ or NR₆;
R₆ is a hydrogen, a lower alkyl group, an aryl group;
R₇ is a lower alkyl group or an aryl group;
R₈ at each occurrence is independently is a hydrogen, a hydroxyl group, a lower alkyl group, an aryl group, —NO₂, —CH₂—ONO₂ or —CH₂—OH;
n′ and m′ are each independently an integer from 0 to 10; and
o is an integer from 0 to 2.

6. The method of claim 1, wherein the compound of Formula (I) is a compound of Formula (VI), (VI), (VIII), (IX), (X) or (XI); the compound of Formula (II) is a compound of Formula (XII); the compound of Formula (III) is a compound of Formula (XIII), (XIV), (XV), (XVI), (XVII), (XVIII), (XIX) or (XX); the compound of Formula (IV) is a compound of Formula (XXI) or (XXI); and the compound of Formula (V) is a compound of Formula (XXIII), (XXIV), (XXV) or (XXVI); or a pharmaceutically acceptable salt thereof,
wherein the compound of Formula (VI) is: 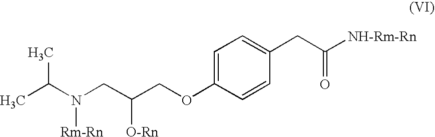
and the compound of Formula (VII) is: 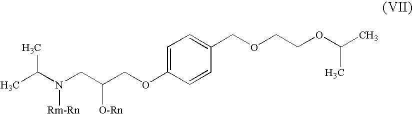
and the compound of Formula (VIII) is: 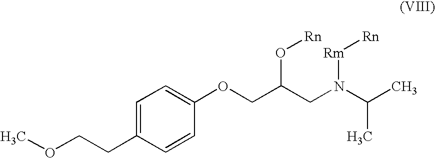
and the compound of Formula (IX) is: 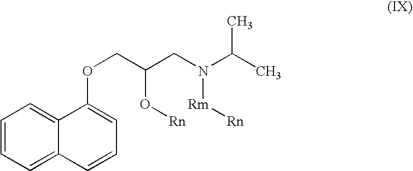
and the compound of Formula (X) is: 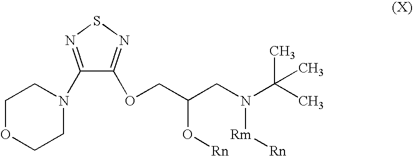
and the compound of Formula (XI) is: 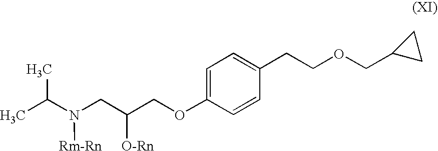
and the compound of Formula (XII) is: 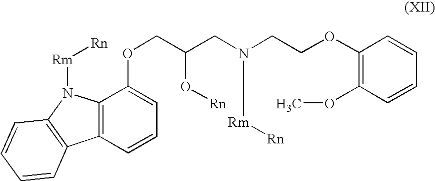
and the compound of Formula (XIII) is: 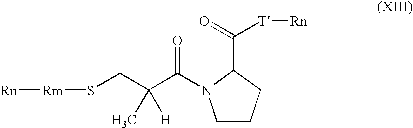
and the compound of Formula (XIV) is: 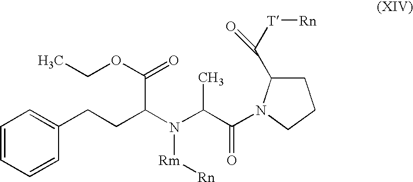
and the compound of Formula (XV) is: 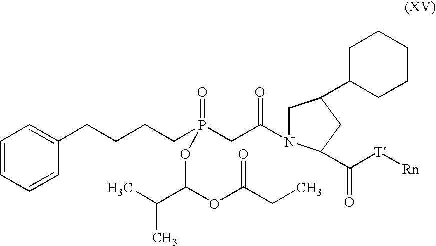
and the compound of Formula (XVI) is: 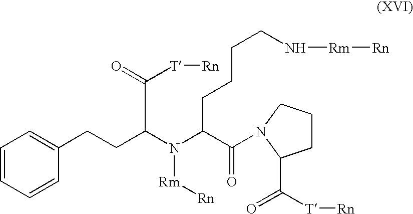
and the compound of Formula (XVII) is: 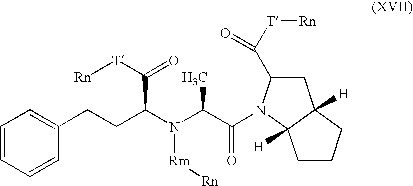
and the compound of Formula (XVIII) is: 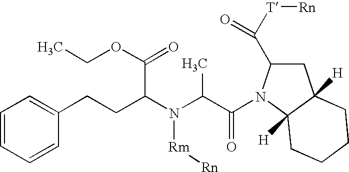
and the compound of Formula (XIX) is: 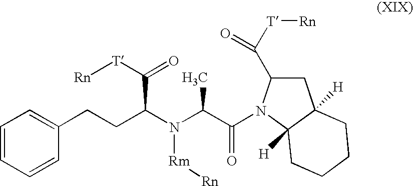
and the compound of Formula (XX) is: 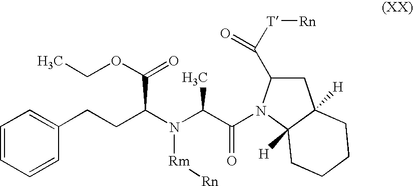
and the compound of Formula (XXI) is: 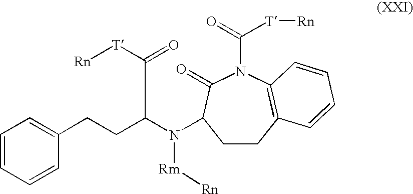
and the compound of Formula (XXII) is: 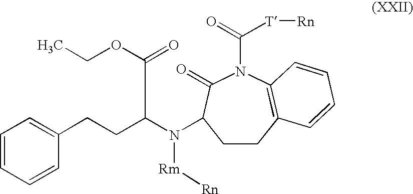
and the compound of Formula (XXII) is: 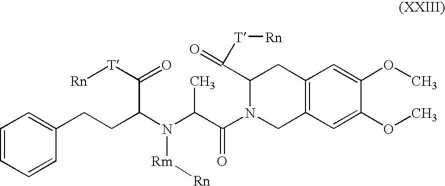
and the compound of Formula (XXIV) is: 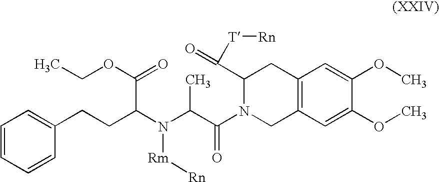
and the compound of Formula (XXV) is: 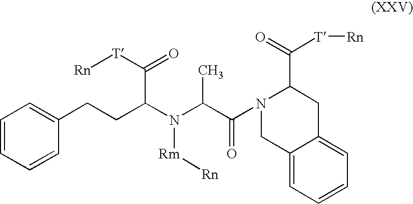
and the compound of Formula (XXVI) is: 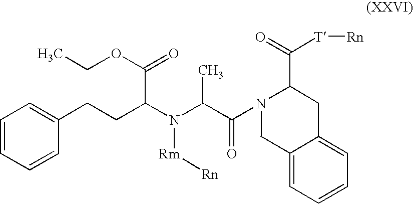
wherein
T′ is oxygen, sulfur or NR₆;
R₆ is a hydrogen, a lower alkyl group, an aryl group;
Rₘ-Rₙ taken together can be a hydrogen atom; or
Rₘ is:
(i) —C—(O)—;
(ii) —C—(O)—NR₆;
(iii) —C(O)—O—;
(iv) —C(O)—S;
(v) —CH₂—O—; or
(vi) —CH(CH₃)—O—;
Rₙ is:
a hydrogen or 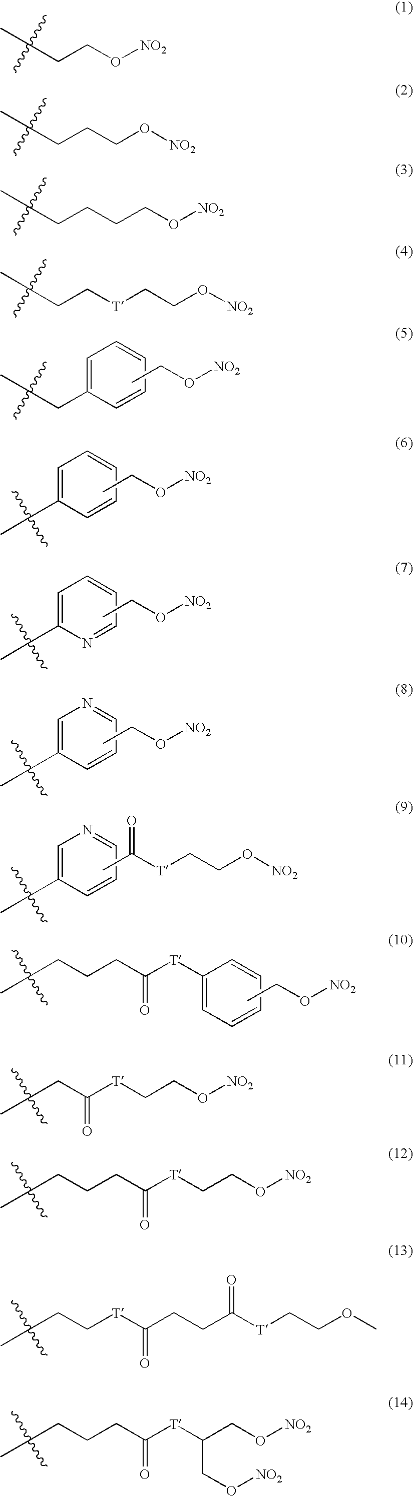 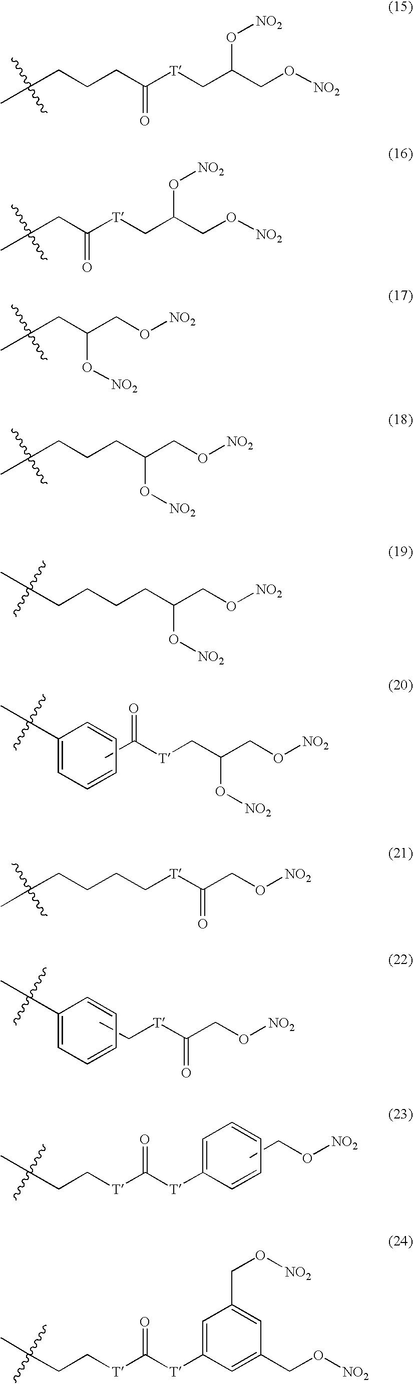 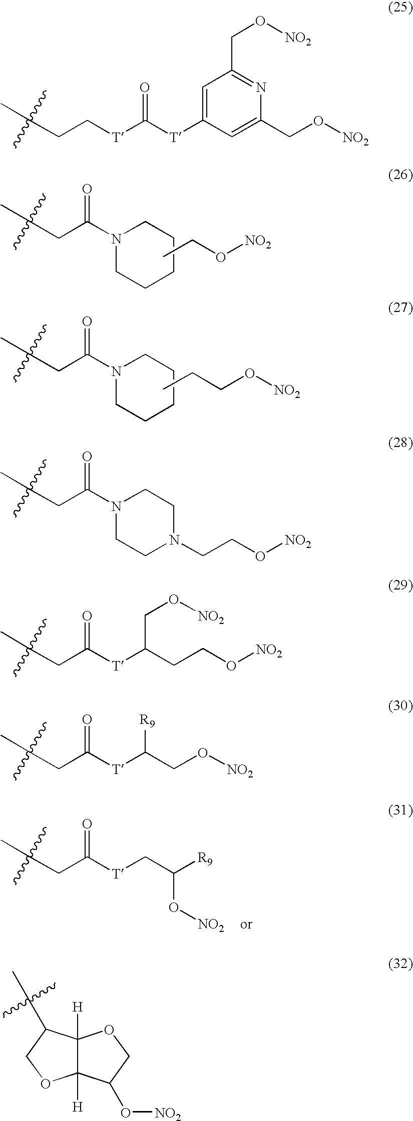
wherein:
R₉ is a lower alkyl group;
T′ is oxygen, sulfur or NR₆;
R₆ is a hydrogen, a lower alkyl group, an aryl group; and
with the proviso that the compounds of Formula (IV) to Formula (XXVI) must contain at least one —NO₂ group.

7. The method of calim 1 wherein the compound of Formula (III) is ethyl (2S)-2-(((1S)-2-((2S)-2-(((2S,6R)-6-(nitrooxy)-4,8-dioxabicyclo(3.3.0)oct-2-yl)oxycarbonyl)pyrrolidinyl)-1-methyl-2-oxoethyl)amino)-4-phenylbutanoate; (2S)-1-((2S)-2-(((1S)-1-(((2S,6R)-6-(nitrooxy)-4,8-dioxabicyclo(3.3.0)oct-2-yl)oxycarbonyl)-3-phenylpropyl)amino)propanoyl)pyrrolidine-2-carboxylic acid; (2S,6R)-6-(nitrooxy)-4,8-dioxabicyclo(3.3.0)oct-2-yl (2S)-2-(((1S)-2-((2S)-2-(((2S,6R)-6-(nitrooxy)-4,8-dioxabicyclo(3.3.0)Oct-2-yl)oxycarbonyl)pyrrolidinyl)-1-methyl-2-oxoethyl)amino)-4-phenylbutanoate or a pharmaceutically acceptable salt thereof:

8. The method of claim 1, wherein the ophthalmic disorder is an ophthalmic infection, a cataract, glaucoma, elevated intraocular pressure, ocular pain a dry eye disorder, ocular hypertension, ocular bleeding, a retinal disease, presbyopia, macular degeneration, choroidal neovascularization, a retinopathy or a retinitis.

9. The method of claim 8, wherein the ophthalmic disorder is an ophthalmic infection, glaucoma, ocular pain following corneal surgery, dry eye disorder, ocular hypertension, ocular bleeding, retinal diseases or disorders, or elevated intraocular pressure.

10. The method of claim 8, wherein the ophthalmic infection is an inflammation of the conjunctiva, an inflammation of the cornea or a corneal ulcer.

11. The method of claim 2, further comprising (i) at least one therapeutic agent; (ii) at least one nitric oxide donor compound; or (iii) at least one therapeutic agent and at least one nitric oxide donor compound.

12. The method of claim 11, wherein the therapeutic agent is an α-adrenergic receptor agonist, an α-adrenergic receptor antagonist, an angiotensin-converting enzyme (ACE) inhibitor, an antimicrobial compound, an antioxidant, a β-adrenergic antagonist, a carbonic anhydrase inhibitor, a hydralazine compound, a nonsteroidal antiinflammatory compound, a prostaglandin, a selective cyclooxygenase-2 inhibitor or a combination of two or more thereof.

13. The method of claim 12, wherein the therapeutic agent is at least one compound selected from the group consisting of an α-adrenergic receptor agonist, an angiotensin-converting enzyme (ACE) inhibitor, an antimicrobial compound, a β-adrenergic antagonist, a carbonic anhydrase inhibitor, a nonsteroidal antiinflammatory compound, a prostaglandin, a selective cyclooxygenase-2 (COX-2) inhibitor and a steroid.

14. The method of claim 11, wherein the nitric oxide donor compound is selected from the group consisting of a S-nitrosothiol, a nitrite, a nitrate, a S-nitrothiol, a sydnonimine, a NONOate, a N-nitrosoamine, a N-hydroxyl nitrosamine, a nitrosimine, a diazetine dioxide, an oxatriazole 5-imine, an oxime, a hydroxylamine, a N-hydroxyguanidine, a hydroxyurea and a furoxan.

15. The method of claim 11, wherein the nitric oxide donor compound is a compound that stimulates the endogenous production of nitric oxide or endothelium-derived relaxing factor in vivo, a compound that elevates endogenous levels of nitric oxide, a compound that is oxidized to produce nitric oxide, a compound that is a substrate for nitric oxide synthase or a compound that is a substrate for cytochrome P450.
